(19) United States
(12) Patent Application Publication
(10) Pub. No.: US 2024/0407395 A1
(43) Pub. Date: Dec. 12, 2024

(54) OPTIMIZED WATER LINE DELIVERY OF MICROBES

(71) Applicants: INTERNATIONAL N&H DENMARK APS, Kongens Lyngby (DK); NUTRITION & BIOSCIENCES USA 4, INC., Wilmington, DE (US)

(72) Inventors: Michael DAUNER, HOCKESSIN, DE (US); Charlotte Horsmans POULSEN, Brabrand (DK); Cathy E. KALBACH, Bear, DE (US)

(21) Appl. No.: 18/695,603

(22) PCT Filed: Sep. 28, 2022
(86) PCT No.: PCT/US2022/045112

§ 371 Date: Mar. 26, 2024

Related U.S. Application Data
(60) Provisional application No. 63/249,989, filed on Sep. 29, 2021.

(51) Int. Cl. A23K 10/18; A01G 25/00; A01K 9/00; A23K 20/163; A23K 50/10; A23K 50/30; A23K 50/75; C02F 1/50; C02F 1/66

(52) U.S. Cl. CPC A23K 10/18; A01G 25/00; A01K 9/005; A23K 20/163; A23K 50/10; A23K 50/30; A23K 50/75; C02F 1/50; C02F 1/66; C02F 2303/04; C02F 2303/185

(57) ABSTRACT

Provided herein, inter alia, are compositions, methods, and kits for optimally delivering viable microorganisms (such as beneficial microbes) through a water line. The compositions are constituted in such a manner as to minimize settling of the microorganisms within the water line and to maximize the survival of the microorganisms during storage and water line transit.

### CROSS-REFERENCE TO RELATED APPLICATIONS

This application claims priority to U.S. Provisional Patent Application No. 63/249,989, filed on Sep. 29, 2021, the disclosure of which is incorporated by reference herein in its entirety.

### FIELD OF THE INVENTION

Provided herein, inter alia, are compositions and methods for uniformly delivering direct fed microbials (DFMs) over distances with reduced losses and without or with decreased settling in water lines.

### BACKGROUND

Over the past several years, there has been increasing governmental and consumer pressure applied to the animal feed industry to decrease or curtail the use of antibiotics as components of animal nutrition feeding regimens. In the poultry industry, for example, live beneficial microbes (a.k.a. probiotics or direct fed microbials (DFMs)) are desired to reduce pathogens and improve growth performance. Major possible routes for administration of these microbes are either through feed or through a water line. However, administration via feed often requires live cells to be formulated in a pellet mill, where harsh conditions can lead to significantly reduced live cell numbers, especially in the case of non-spore forming microorganisms. Hence, distribution of microbes via water line appears more convenient and attractive, as conditions are less detrimental to cell survival.

However, there is only very limited mixing of water in a water line and waterflow at the end of the line is low and generally dependent on the drinking activity of livestock. Up to several hours can be required for the microbes to completely transit the water line drinking system of a livestock-raising facility from the location where the microbe is introduced into the drinking system to the last nipple drinkers on the water line. Since cells have a higher specific density than water, but often have no way of active movement on their own (e.g. flagella and/or sufficient energy to operate them), they settle. If their settling velocity exceeds a certain threshold, none of the cells will arrive at the end of the water line, and only livestock at the beginning of the water line will receive live cells, leading to non-homogenous dosing. Moreover, all cells settling to the bottom of the line will not provide any benefit to the livestock, leading to significant product loss and potential fouling of the water line.

What is needed, therefore, are compositions and methods for optimally delivering microbes through a water line over a distance without attendant settling of the microbes prior to reaching the end of the line.

The subject matter disclosed herein addresses these needs and provides additional benefits as well.

### SUMMARY

Provided herein, inter alia, are compositions, methods, and kits for optimally delivering viable microorganisms to livestock or other living organisms (such as, without limitation, plants, for example, commercially grown crops) through a water line. The compositions are constituted in such a manner as to minimize settling of the microorganisms within the water line and to maximize the survival of the microorganisms during water line transit.

In some aspects, provided herein are compositions comprising a) a buffer sufficient to maintain pH of water at about or greater than about 6.5; and b) one or more direct fed microbials (DFMs). In some embodiments, the composition further comprises c) a thickening agent. In some embodiments, the thickening agent is xanthan gum. In some embodiments of any of the embodiments disclosed herein, the DFMs are freeze dried or lyophilized. In some embodiments, the DFMs comprise a cryoprotectant. In some embodiments of any of the embodiments disclosed herein, the composition is hydrated. In some embodiments of any of the embodiments disclosed herein, the buffer is sufficient to maintain pH of water in a water line at about or greater than about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments of any of the embodiments disclosed herein, the buffer is sufficient to maintain pH of water in a water line at about or greater than about 6.5. In some embodiments of any of the embodiments disclosed herein, the concentration of the buffer (e.g., the hydrated buffer) is about 1-2000 mM. In some embodiments, the composition comprises about 320 mM of the buffer (or a buffer to yield of about 320 mM after hydration). In some embodiments, the composition comprises about 0.001-20 mM of the buffer (or a buffer to yield of about 0.001-20 mM in the waterline). In some embodiments, the composition comprises about 2.5 mM buffer (or a buffer to yield of about 2.5 mM in the waterline). In some embodiments of any of the embodiments disclosed herein, the composition comprises about 0.1% w/w xanthan gum. In some embodiments of any of the embodiments disclosed herein, the buffer comprises one or more buffers selected from the group consisting of potassium phosphate, carbonate, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid. In some embodiments of any of the embodiments disclosed herein, the DFM comprises one or more DFM selected from the group consisting of bacteria, algae, and fungi. In some embodiments, the bacteria comprises a gram-positive bacteria or a gram-negative bacteria. In some embodiments, the fungi comprises a yeast. In some embodiments of any of the embodiments disclosed herein, the bacteria is one or more bacteria selected from the group consisting of a Bacillus spp., a Bifidobacterium spp., a Lactobacillus spp., and a Megasphaera spp. In some embodiments, the bacteria is one or more bacteria selected from the group consisting of Bacillus subtilis, Bacillus amyloliquefaciens, Bacillus velezensis, Bifidobacterium animalis subsp. lactis, Lactobacillus reuteri. Lactobacillus acidophilus, and Megasphaera elsdenii. In some embodiments of any of the embodiments disclosed herein, the DFMs exhibit decreased settling and/or improved survival in the hydrated composition compared to identical DFMs that are not in a hydrated composition comprising a buffer sufficient to maintain pH of water at about or greater than about 6.5. In some embodiments of any of the embodiments disclosed herein, the water is selected from the group consisting of a municipal water source, well water, surface water, and collected rain water. In some embodiments, the water is supplemented with chloramine or other chlorine-based antimicrobial. In some embodiments, the composition further comprises d) means for inactivating chloramine or chlorine present in the water. In some embodiments, the composition further comprises e) a wetting agent and/or dispersing agent. In some embodiments of any of the embodiments disclosed herein, the temperature of the composition is less than about 15° C.

In other aspects, provided herein are methods for preparing a composition for maximizing delivery of live direct fed microbial (DFM) cells through a water line comprising mixing i) a buffer sufficient to maintain pH of water at about or greater than about 6.5; and ii) one or more direct fed microbials (DFMs) with water. In some embodiments, the DFMs are freeze dried or lyophilized. In some embodiments, the DFMs comprise one or more cryoprotectant. In some embodiments of any of the embodiments disclosed herein, the water is selected from the group consisting of a municipal water source, well water, surface water, and collected rain water. In some embodiments of any of the embodiments disclosed herein, the method further comprises mixing iii) a thickening agent with water. In some embodiments of any of the embodiments disclosed herein, the buffer is sufficient to maintain pH of water in a water line at about or greater than about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments of any of the embodiments disclosed herein, the mixing results in a final concentration of about 1-2000 mM of the buffer. In some embodiments, the mixing results in a final concentration of about 320 mM of the buffer. In some embodiments of any of the embodiments disclosed herein, the mixing results in a final concentration of about 0.1% w/w thickening agent. In some embodiments of any of the embodiments disclosed herein, the buffer is one or more buffers selected from the group consisting of potassium phosphate, carbonate, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid. In some embodiments of any of the embodiments disclosed herein, the DFM is one or more DFM selected from the group consisting of bacteria and fungi. In some embodiments, the bacteria is a gram-positive bacteria or a gram-negative bacteria. In some embodiments, the fungi is a yeast. In some embodiments of any of the embodiments disclosed herein, the bacteria is one or more bacteria selected from the group consisting of a Bacillus spp., a Bifidobacterium spp., a Lactobacillus spp., and a Megasphaera spp. In some embodiments, the bacteria is one or more bacteria selected from the group consisting of Bacillus subtilis, Bacillus amyloliquefaciens, Bacillus velezensis, Bifidobacterium animalis subsp. lactis, Lactobacillus reuteri. Lactobacillus acidophilus, and Megasphaera elsdenii. In some embodiments of any of the embodiments disclosed herein, the water is supplemented with chloramine or other chlorine-based antimicrobial. In some embodiments of any of the embodiments disclosed herein, the method further comprises mixing iv) means for inactivating chloramine or chlorine present in the water with water. In some embodiments of any of the embodiments disclosed herein, the method further comprises mixing v) a wetting agent and/or dispersing agent with water. In some embodiments of any of the embodiments disclosed herein, the temperature of the composition is less than about 15° C.

In still further aspects, provided herein is a water line composition produced by any of the methods disclosed herein.

In yet another aspect, provided herein are methods for delivering one or more direct fed microbials (DFMs) to a subject comprising administering any of the compositions disclosed herein through a water line over a distance, wherein the DFMs are delivered to the subject when the subject drinks from the water line. In some embodiments, the DFMs exhibit decreased settling and/or survival in the water line compared to identical DFMs that are not administered any of the compositions disclosed herein. In some embodiments of any of the embodiments disclosed herein, the water line comprises nipple drinkers. In some embodiments of any of the embodiments disclosed herein, the distance is between about 20 meters to about 200 meters. In some embodiments, the water line further comprises between about 20-100 meters of piping that lacks nipple drinkers. In some embodiments of any of the embodiments disclosed herein, the subject is poultry, swine, or a ruminant. In some embodiments, the poultry is a chicken or a turkey. In some embodiments of any of the embodiments disclosed herein, the chicken is a layer or a broiler. In some embodiments, the swine is a piglet, a grower, a finisher, or a sow. In some embodiments, the ruminant is a beef cattle, a milk cattle, or a veal cattle.

In additional aspects, provided herein is a kit comprising a) a buffer sufficient to maintain pH of water at about or greater than about 6.5; and b) one or more direct fed microbials (DFMs). In some embodiments, the kit further comprises c) a thickening agent, for example, xanthan gum. In some embodiments of any of the embodiments disclosed herein, the buffer is sufficient to maintain pH of water in a water line at about or greater than about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments of any of the embodiments disclosed herein, the kit comprises 3-500 g of the buffer. In some embodiments of any of the embodiments disclosed herein, the buffer comprises one or more buffers selected from the group consisting of potassium phosphate, carbonate, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid. In some embodiments of any of the embodiments disclosed herein, the DFM comprises one or more DFM selected from the group consisting of bacteria, algae, and fungi. In some embodiments, the bacteria comprises a gram-positive bacteria or a gram-negative bacteria. In some embodiments, the fungi comprises a yeast. In some embodiments of any of the embodiments disclosed herein, the bacteria is one or more bacteria selected from the group consisting of a Bacillus spp., a Bifidobacterium spp., a Lactobacillus spp., and a Megasphaera spp. In some embodiments, the bacteria is one or more bacteria selected from the group consisting of Bacillus subtilis, Bacillus amyloliquefaciens, Bacillus velezensis, Bifidobacterium animalis subsp. lactis, Lactobacillus reuteri, Lactobacillus acidophilus, and Megasphaera elsdenii. In some embodiments of any of the embodiments disclosed herein, the kit further comprises d) means for inactivating chloramine or chlorine present in water. In some embodiments of any of the embodiments disclosed herein, the kit components are packaged in a sachet, water-dissolvable tablets, water-dissolvable pods (e.g. powder distributed in cold water soluble PVA film bags) or in water-dissolvable (soft gel) capsules (e.g. in pullulan capsules). In some embodiments of any of the embodiments disclosed herein, the kit further comprises e) written instructions for combining the kit components with water for water line delivery. In some embodiments of any of the embodiments disclosed herein, the DFMs are freeze dried or lyophilized. In some embodiments, the DFMs further comprise a cryoprotectant. In some embodiments of any of the embodiments disclosed herein, the kit further comprises f) a wetting and/or dispersing agent.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts a graph showing normalized optical density measurements with Lactobacillus reuteri strain LAB1 at initial OD₆₀₀ value of 0.9976 (A0=1.000, black solid line), 0.8456 (A0=0.800, dark grey solid line), 0.6670 (A0=0.600, light grey solid line), 0.4834 (A0=0.400, black dotted line), 0.2872 (A0=0.200, dark grey dotted line), 0.1858 (A0=0.100, light grey dotted line), 0.1620 (A0=0.080, black striped line), 0.1476 (A0=0.060, dark grey striped line) and 0.1295 (A0=0.040, light grey striped line) concentrations for 48 h.

FIG. 2 depicts a graph showing normalized optical density measurements with freeze-dried Lactobacillus reuteri LAB-1 powder in 100 mM phosphate buffer mono-/dipotassium solution of various ratios with a resulting pH of 7.87 (black solid line), 7.32 (dark grey solid line), 6.92 (light grey solid line), 6.51 (black striped line) and 5.92 (dark grey striped line) at 4° C. over 48 h.

FIG. 3 depicts a graph showing normalized optical density measurements with freeze-dried Lactobacillus reuteri CMP19 powder in 100 mM phosphate buffer mono-/dipotassium solution of various ratios with a resulting pH of 8.48 (black solid line), 6.70 (black long-striped line), 6.28 (black short-striped line) and 4.81 (black dotted line) at 4° C. over 5800 min.

FIG. 4 depicts a graph showing normalized optical density measurements with freeze-dried Lactobacillus reuteri CMP21 powder in 100 mM phosphate buffer mono-/dipotassium solution of various ratios with a resulting pH of 8.59 (black solid line), 6.70 (black long-striped line), 6.17 (black short-striped line) and 4.81 (black dotted line) at 4° C. over 5800 min.

FIG. 5 depicts a graph showing normalized optical density measurements with freeze-dried three strain Lactobacillus reuteri consortium (Con-S) powder in 100 mM phosphate buffer mono-/dipotassium solution of various ratios with a resulting pH of 8.49 (black solid line), 6.57 (black long-striped line), 6.19 (black short-striped line) and 4.68 (black dotted line) at 4° C. over 5800 min.

FIG. 6 depicts a graph showing normalized optical density measurements with freeze-dried Lactobacillus reuteri LAB-1 powder in 100 mM phosphate buffer mono-/dipotassium solution of various ratios with a resulting pH of 4.00 (black solid line), 4.70 (dark grey solid line), 5.79 (light grey solid line), 6.86 (long-striped black line), 7.67 (long-striped dark grey line) and 8.14 (long-striped light grey line) at 23° C. over 2880 min.

FIG. 7 depicts a graph showing normalized optical density measurements with freeze-dried Lactobacillus reuteri LAB-1 powder in phosphate buffer mono-/dipotassium solution at constant ratio but various molarities with a resulting pH of 6.86 at 100 mM (black solid line), 6.86 at 50 mM (dark grey solid line), 6.87 at 10 mM (light grey solid line), 6.84 at 5 mM (long-striped black line) and 6.65 at 1 mM (long-striped dark grey line) at 23° C. over 2880 min.

FIG. 8 depicts a graph showing normalized optical density measurements with freeze-dried Lactobacillus reuteri LAB-1 powder in 100 mM phosphate buffer mono-/dipotassium solution of various ratios with a resulting pH of 8.66 (black solid line), 6.80 (dark grey solid line), 6.67 (light grey solid line), 6.58 (black dotted line), 6.52 (dark grey dotted line), 6.37 (light grey dotted line), 6.25 (black short-striped line), 6.15 (dark grey short-striped line), 5.99 (light grey short-striped line), 5.84 (black long-striped line) and 5.53 (dark grey long-striped line) at 23° C. over 2880 min.

FIG. 9 depicts a graph showing normalized optical density measurements with freeze-dried Lactobacillus reuteri CMP19 powder in 100 mM phosphate buffer mono-/dipotassium solution of various ratios with a resulting pH of 8.48 (pH8, black solid line), 6.70 (pH7, black long-striped line), 6.28 (pH6, black short-striped line) and 4.81 (pH5, black dotted line) at 23° C. over 7000 min.

FIG. 10 depicts a graph showing normalized optical density measurements with freeze-dried Lactobacillus reuteri CMP21 powder in 100 mM phosphate buffer mono-/dipotassium solution of various ratios with a resulting pH of 8.59 (pH8, black solid line), 6.70 (pH7, black long-striped line), 6.17 (pH6, black short-striped line) and 4.69 (pH5, black dotted line) at 23° C. over 7000 min.

FIG. 11 depicts a graph showing normalized optical density measurements with a powder of a freeze-dried consortium containing three of Lactobacillus reuteri strains in 100 mM phosphate buffer mono-/dipotassium solution of various ratios with a resulting pH of 8.49 (pH8, black solid line), 6.57 (pH7, black long-striped line), 6.19 (pH6, black short-striped line) and 4.68 (pH5, black dotted line) at 23° C. over 7000 min.

FIG. 12 depicts a graph showing normalized optical density measurements with freeze-dried Lactobacillus reuteri LAB1 powder in 100 mM phosphate buffer mono-/dipotassium solution of various ratios with a resulting pH of 7.75 (pH=7.75, black solid line), 7.24 (pH=7.24, dark grey solid line), 6.83 (pH=6.83, light grey solid line), 6.43 (pH=6.43, black striped line) and 5.84 (pH=5.84, dark grey striped line) at 37° C. over 48 h.

FIG. 13 depicts normalized optical density measurements with freeze-dried LAB-1 powder in 100 mM mono-/disodium carbonate buffer solution of various ratios with a resulting initial pH of 10.23 (black solid line), 9.87 (dark grey solid line), 9.49 (light grey solid line), 8.37 (dotted black line), 8.25 (dotted dark grey line), 8.15 (dotted light grey line), 7.87 (black striped line) and 6.01 (dark grey striped line) at 23° C. over 2900 min.

FIG. 14 depicts normalized optical density measurements with freeze-dried Lactobacillus reuteri LAB-1 powder in 100 mM mono-/disodium carbonate buffer solution of various ratios with a resulting initial pH of 10.23 (black solid line), 7.87 (black dotted line) and 6.01 (black striped line). In addition, settling curve of a 10 mM mono-/disodium carbonate buffer solution at pH=10.60 (solid light grey line) and of a 1 mM mono-/disodium carbonate buffer solution at pH=7.75 (dotted light grey line) at 23° C. over 2900 min is displayed.

FIG. 15 depicts normalized optical density measurements with S. cerevisiae (from Zenith yeast concentrate 6400, AB Biotek, St. Louis, MO) in a 100 mM phosphate buffer mono-/dipotassium solution of various ratios with a resulting pH of 4.41 (pH5, black solid line), 6.32 (pH6, dotted black line), 6.69 (pH7, short-striped black line), and 8.74 (pH8, long-striped black line) over 48 h.

FIG. 16 depicts a graph showing normalized optical density measurements with freeze-dried Bifidobacterium lactis BI-04® powder in 100 mM phosphate buffer mono-/dipotassium solution of various ratios with a resulting pH of 4.41 (pH5, black solid line), 6.32 (pH6, dotted black line), 6.69 (pH7, short-striped black line), and 8.74 (pH8, long-striped black line) over 48 h.

FIG. 17 depicts a graph showing normalized optical density measurements with freeze-dried Lactobacillus plantarum Lp115 (Danisco, USA) powder in 100 mM phosphate buffer mono-/dipotassium solution of various ratios with a resulting pH of 4.66 (pH5, black solid line), 6.22 (pH6, dotted black line), 6.64 (pH7, short-striped black line), and 8.47 (pH8, long-striped black line) over 48 h.

FIG. 18 depicts a graph showing normalized optical density measurements with freeze-dried Lactobacillus reuteri LAB-1 powder in 100 mM phosphate buffer mono-/dipotassium solution of various ratios with a resulting pH of 8.42 (black solid line), 7.04 (dark grey solid line), 6.54 (light grey solid line), 6.05 (black dotted line), 5.58 (dark grey dotted line), 5.20 (light grey dotted line) and 4.63 (black striped line) at 23° C. over 48 h.

FIG. 19 depicts a graph showing the Zeta-potential of freeze-dried Lactobacillus reuteri LAB-1 powder in 100 mM phosphate buffer mono-/dipotassium solution of various ratios with resulting pHs of 8.42, 7.04, 6.54, 6.05, 5.58, 5.20 and 4.63 (measurement points) at 23° C. The error interval indicates the standard error of the mean from 3 repeat measurements.

FIG. 20 depicts a graph showing the Zeta-potential of freeze-dried Lactobacillus reuteri CMP-19 powder in 2.5 mM phosphate buffer mono-/dipotassium solution of various ratios with resulting pHs of 5.66, 5.85, 5.91, 6.03, 6.19, 6.39, 6.58, 6.77, 6.92, 7.02 and 7.16 (measurement points) at 23° C. The error interval indicates the standard error of the mean from 3 repeat measurements.

FIG. 21 depicts a graph showing the Zeta-potential of freeze-dried Lactobacillus reuteri CMP-21 powder in 2.5 mM phosphate buffer mono-/dipotassium solution of various ratios with resulting pHs of 6.10, 6.08, 6.11, 6.23, 6.39, 6.61, 6.80, 6.96, 7.09, 7.20 and 7.35 (measurement points) at 23° C. The error interval indicates the standard error of the mean from 3 repeat measurements.

FIG. 22 depicts a graph showing the Zeta-potential of the three-strain Lactobacillus reuteri-containing Consortium-S(ConS) in 2.5 mM phosphate buffer mono-/dipotassium solution of various ratios with resulting pHs of 4.57, 4.80, 4.92, 5.09, 5.29, 5.47, 5.63, 7.13, 7.22, and 7.38 (measurement points) at 23° C. The error interval indicates the standard error of the mean from 3 repeat measurements.

FIG. 23 depicts a graph showing normalized optical density measurements with freeze-dried Lactobacillus reuteri LAB-1 powder in 100 mM BIS TRIS buffer solutions of different pH as adjusted by HCl addition in initial pH values of 6.98 (solid line), 6.47 (long-striped line), 5.98 (short-striped line) and 5.53 (dotted line) at 23° C. over 2890 min.

FIG. 24 depicts a graph showing normalized optical density measurements with freeze-dried Lactobacillus reuteri LAB-1 powder in 100 mM phosphate buffer solutions of different pH and different alginate concentrations at 23° C. over 48 h. In specific: 0.1% (w/v) alginate (“0.1% Scogin, pH=5.77”, black dotted line), 0.1% % (w/v) alginate (“0.1% Scogin, pH=6.85”, light grey dotted line), 0.3% (w/v) alginate (“0.3% Scogin, pH=5.81”, striped black line), 0.3% % (w/v) alginate (“0.3% Scogin, pH=6.89”, light grey striped line), 0.6% (w/v) alginate (“0.6% Scogin, pH=5.78”, solid black line), 0.6% % (w/v) alginate (“0.6% Scogin, pH=6.85”, light grey solid line).

FIG. 25 depicts a graph showing normalized optical density measurements with freeze-dried Lactobacillus reuteri LAB-1 powder in 100 mM phosphate buffer solutions of different pH and different microcrystalline content at 23° C. over 48 h. In specific: 0.1% (w/v) microcrystalline content (“0.1% NTC90, pH=5.65”, black striped line), 0.1% % (w/v) microcrystalline content (“0.1% NTC90, pH=6.87”, light grey striped line), 0.3% (w/v) microcrystalline content (“0.3% NTC90, pH=5.69”, solid black line) and 0.3% % (w/v) microcrystalline content (“0.3% NTC90, pH=6.89”, light grey solid line).

FIG. 26 depicts a graph showing normalized optical density measurements with freeze-dried Lactobacillus reuteri LAB-1 powder in 100 mM phosphate buffer solutions of different pH and different xanthan gum content at 23° C. over 48 h. In specific: PB, pH=5.65: dark grey solid line, PB, pH=6.91: black solid line, PB+0.1% (w/v) xanthan gum, pH=5.55: dark grey striped line, PB+0.1% (w/v) xanthan gum, pH=6.89: black striped line, PB+0.3% (w/v) xanthan gum, pH=5.57: dark grey dotted line, and PB+0.3% (w/v) xanthan gum, pH=6.87: black dotted line.

FIG. 27 depicts a graph showing normalized optical density measurements with freeze-dried Megasphaera elsdenii powder in 100 mM phosphate buffer mono-/dipotassium solution of various ratios with a resulting pH of 8.69 (black solid line) and 6.88 (dark grey solid line) over 48 h.

FIG. 28 depicts a graph showing normalized optical density measurements with freeze-dried Lactobacillus reuteri LAB-1 powder in 100 mM phosphate buffer mono-/dipotassium solution of various ratios with a resulting pH of 8.53 (black solid line), 6.74 (dark grey solid line), 6.34 (light grey solid line) and 4.64 (black dotted line) at 14° C. over 5250 min.

FIG. 29 depicts a graph showing normalized optical density measurements with freeze-dried Lactobacillus reuteri CMP19 powder in 100 mM phosphate buffer mono-/dipotassium solution of various ratios with a resulting pH of 8.43 (black solid line), 6.71 (dark grey solid line), 6.27 (light grey solid line) and 4.80 (black dotted line) at 14° C. over 5250 min.

FIG. 30 depicts a graph showing normalized optical density measurements with freeze-dried Lactobacillus reuteri CMP21 powder in 100 mM phosphate buffer mono-/dipotassium solution of various ratios with a resulting pH of 8.70 (black solid line), 6.74 (dark grey solid line), 6.27 (light grey solid line) and 4.65 (black dotted line) at 14° C. over 5250 min.

FIG. 31 depicts a graph showing normalized optical density measurements with freeze-dried Lactobacillus reuteri LAB-1 powder in 100 mM phosphate buffer mono-/dipotassium solution of various ratios with a resulting pH of 8.53 (black solid line), 6.74 (dark grey solid line), 6.34 (light grey solid line) and 4.64 (black dotted line) at 30° C. over 7290 min.

FIG. 32 depicts a graph showing normalized optical density measurements with freeze-dried Lactobacillus reuteri CMP19 powder in 100 mM phosphate buffer mono-/dipotassium solution of various ratios with a resulting pH of 8.43 (black solid line), 6.71 (dark grey solid line), 6.27 (light grey solid line) and 4.80 (black dotted line) at 30° C. over 7290 min.

FIG. 33 depicts a graph showing normalized optical density measurements with freeze-dried Lactobacillus reuteri CMP21 powder in 100 mM phosphate buffer mono-/dipotassium solution of various ratios with a resulting pH of 8.70 (black solid line), 6.74 (dark grey solid line), 6.27 (light grey solid line) and 4.65 (black dotted line) at 30° C. over 7290 min.

FIG. 34 depicts a graph showing normalized optical density measurements with freeze-dried Lactobacillus reuteri CMP19 powder in 100 mM phosphate buffer mono-/dipotassium solution of various ratios with a resulting pH of 8.48 (black solid line), 6.40 (dark grey solid line), 6.28 (light grey solid line) and 4.81 (black dotted line) at 37° C. over 4700 min.

FIG. 35 depicts a graph showing normalized optical density measurements with freeze-dried Lactobacillus reuteri CMP21 powder in 100 mM phosphate buffer mono-/dipotassium solution of various ratios with a resulting pH of 8.59 (black solid line), 6.70 (dark grey solid line), 6.17 (light grey solid line) and 4.69 (black dotted line) at 37° C. over 4700 min.

FIG. 36 depicts a graph showing normalized optical density measurements with freeze-dried three strain Lactobacillus reuteri consortium (Con-S) powder in 100 mM phosphate buffer mono-/dipotassium solution of various ratios with a resulting pH of 8.49 (black solid line), 6.57 (dark grey solid line), 6.19 (light grey solid line) and 4.68 (black dotted line) at 37° C. over 4700 min.

FIG. 37 depicts a bar graph showing viable cell concentration in [MPN/g] of freeze-dried powder of a three-strain Lactobacillus reuteri-containing Consortium-S(ConS) as determined by MPN analysis blended with different ratios of mono- or dipotassium phosphate (PB), or mono- and disodium bicarbonate salts (CB) after resuspension in 14° C. ultrapure water (T0), and after storage of 1 month (T1m), 2 months (T2m) at 4° C., 2 months (T2m) or 12 months (T12m) at 25° C. and resuspension in 14° C. ultrapure water.

FIG. 38 depicts a bar graph showing viable cell concentration in [MPN/g] of freeze-dried powder of a three-strain Lactobacillus reuteri-containing Consortium-S(ConS) as determined by MPN analysis blended with different ratios of mono- or dipotassium phosphate (PB), or mono- and disodium bicarbonate salts (CB) after resuspension in 14° C. ultrapure water (T0), and after storage of 1 month (T1m) or 2 months (T2m) at 25° C.

FIG. 39 depicts a bar graph showing viable cell concentration in [MPN/g] of freeze-dried powder of a three-strain Lactobacillus reuteri-containing Consortium-S(ConS) as determined by MPN analysis blended with different ratios of mono- or dipotassium phosphate (PB), or mono- and disodium bicarbonate salts (CB) after resuspension in 14° C. ultrapure water (T0), and after storage of 1 month (Tim), 2 months (T2m), or 12 months (T12m) at 37° C. and resuspension in 14° C. ultrapure water.

FIG. 40 depicts a bar graph showing viable cell concentration in [MPN/g] of freeze-dried powder of a three-strain Lactobacillus reuteri-containing Consortium-S(ConS) as determined by MPN analysis blended with different ratios of mono- or dipotassium phosphate (PB), or mono- and disodium bicarbonate salts (CB) after resuspension in 14° C. ultrapure water (T0), and after storage of 1 month or 2 months at 4° C., resuspension and incubation in 14° C. ultrapure water, yielding results T1m+4 h and T2m+4 h, respectively.

FIG. 41 depicts a bar graph showing viable cell concentration in [MPN/g] of freeze-dried powder of a three-strain Lactobacillus reuteri-containing Consortium-S(ConS) as determined by MPN analysis blended with different ratios of mono- or dipotassium phosphate (PB), or mono- and disodium bicarbonate salts (CB) after resuspension in 14° C. ultrapure water (T0), and after storage of 1 month or 2 months at 25° C., resuspension and incubation in 14° C. ultrapure water, yielding results T1m+4 h and T2m+4 h, respectively.

FIG. 42 depicts a bar graph showing viable cell concentration in [MPN/g] of freeze-dried powder of a three-strain Lactobacillus reuteri-containing Consortium-S(ConS) as determined by MPN analysis blended with different ratios of mono- or dipotassium phosphate (PB), or mono- and disodium bicarbonate salts (CB) after resuspension in 14° C. ultrapure water (T0), and after storage of 1 month or 2 months at 37° C., resuspension and incubation in 14° C. ultrapure water, yielding results T1m+4 h and T2m+4 h, respectively.

FIG. 43 depicts a bar graph showing viable cell concentration in [MPN/g] of freeze-dried powder of a three-strain Lactobacillus reuteri-containing Consortium-S(ConS) as determined by MPN analysis blended with different amounts of mono- and dipotassium phosphate (PB) or different amounts of monosodium bicarbonate (CB) after resuspension in 14° C. ultrapure water (T0), and after storage of approximately 1 month (T1m), 2 months (T2m) or 12 months (T12m) at 4° C. and subsequent resuspension.

FIG. 44 depicts a bar graph showing viable cell concentration in [MPN/g] of freeze-dried powder of a three-strain Lactobacillus reuteri-containing Consortium-S(ConS) as determined by MPN analysis blended with different amounts of mono- and dipotassium phosphate (PB) or different amounts of monosodium bicarbonate (CB) after resuspension in 14° C. ultrapure water (T0), and after storage of approximately 1 month (Tim), 2 months (T2m) or 12 months (T12m) at 25° C. and subsequent resuspension.

FIG. 45 depicts a bar graph showing viable cell concentration in [MPN/g] of freeze-dried powder of a three-strain Lactobacillus reuteri-containing Consortium-S(ConS) as determined by MPN analysis blended with different amounts of mono- and dipotassium phosphate (PB) or different amounts of monosodium bicarbonate (CB) after resuspension in 14° C. ultrapure water (T0), and after storage of approximately 1 month (Tim), 2 months (T2m) or 12 months (T12m) at 37° C. and subsequent resuspension.

FIG. 46 depicts a bar graph showing viable cell concentration in [MPN/g] of freeze-dried powder of a three-strain Lactobacillus reuteri-containing Consortium-S (ConS) as determined by MPN analysis blended with different amounts of mono- and dipotassium phosphate (PB) or different amounts of monosodium bicarbonate (CB) immediately after resuspension in 14° C. ultrapure water (T0), after resuspension and incubation for 4 h in 14° C. ultrapure water (T0+4 h) and after storage of approximately 1 month, 2 months or 12 months at 4° C., resuspension and incubation for 4 h in 14° C. ultrapure water, yielding results T1m+4 h, T2m+4 h and T12m+4 h, respectively.

FIG. 47 depicts a bar graph showing viable cell concentration in [MPN/g] of freeze-dried powder of a three-strain Lactobacillus reuteri-containing Consortium-S(ConS) as determined by MPN analysis blended with different amounts of mono- and dipotassium phosphate (PB) or different amounts of monosodium bicarbonate (CB) immediately after resuspension in 14° C. ultrapure water (T0), after resuspension and incubation for 4 h in 14° C. ultrapure water (T0+4 h), and after storage of approximately 1 month, 2 months or 12 months at 25° C., resuspension and incubation in 14° C. ultrapure water, yielding results T1m+4 h, T2m+4 h and T12m+4 h, respectively.

FIG. 48 depicts a bar graph showing viable cell concentration in [MPN/g] of freeze-dried powder of a three-strain Lactobacillus reuteri-containing Consortium-S(ConS) as determined by MPN analysis blended with different amounts of mono- and dipotassium phosphate (PB) or different amounts of monosodium bicarbonate (CB) immediately after resuspension in 14° C. ultrapure water (T0), after resuspension and incubation for 4 h in 14° C. ultrapure water (T0+4 h), and after storage of approximately 1 month, 2 months or 12 months at 37° C., resuspension and incubation in 14° C. ultrapure water, yielding results T1m+4 h, T2m+4 h and T12m+4 h, respectively.

FIG. 49 depicts a graph showing concentrations of citrate (solid line with circles), acetate (square dotted line squares), lactate (dashed line with diamonds) as well as pH (round dotted line with triangles) over four hours after rehydration in 14° C. water (“in the bucket”) of the sample stored for approximately 2 months at 4° C. The pH rapidly decreases from its initial value, while citrate, acetate and lactate concentrations increase.

FIG. 50 depicts a graph showing concentrations of citrate (solid line with circles), acetate (square dotted line squares), lactate (dashed line with diamonds) as well as pH (round dotted line with triangles) over four hours after rehydration in 14° C. water (“in the bucket”) of the sample stored for approximately 2 months at 25° C. The pH decreases from its initial value, while citrate, acetate and lactate concentrations increase.

FIG. 51 depicts a graph showing concentrations of citrate (solid line with circles), acetate (square dotted line squares), lactate (dashed line with diamonds) as well as pH (round dotted line with triangles) over four hours after rehydration in 14° C. water (“in the bucket”) of the sample stored for approximately 2 months at 37° C. The pH only slowly decreases from its initial value, while citrate, acetate and lactate concentrations show no or only a small increase.

FIG. 52 depicts a graph showing the Zeta-potentials of (i) the freeze-dried Gram-positive aerotolerant anaerobe Lactobacillus acidophilus NCFM (squares), (ii) the freeze-dried Gram-positive aerotolerant anaerobe Bifidobacterium animalis susp. lactis B1-04 (triangles), (iii) the freeze-dried Gram-negative anaerobe Megasphaera elsdenii 1265 (diamonds), and (iv) the eukaryotic fungi Saccharomyces cerevisiae (circles) from Zenith Yeast Concentrate in dependence of the pH in 100 mM phosphate buffer suspensions.

FIG. 53 depicts a graph showing the change of transmission (AT) and backscattering (ABS) in [%] at different heights of the vial during multiple measurements in the time interval from 0 h to 8 h of a suspension with freeze-dried Lactobacillus reuteri LAB1 dissolved in 100 mM of KH₂PO₄ with pH=4.70.

FIG. 54 depicts a graph showing the change of transmission (AT) and backscattering (ABS) in [%] at different heights of the vial during multiple measurements in the time interval from 0 h to 8 h of a suspension with freeze-dried Lactobacillus reuteri LAB1 dissolved in 100 mM of K₂HPO₄ with pH=8.48.

FIG. 55 depicts one non-limiting embodiment of a workflow/setup for use in a microbial water line delivery system using the methods and compositions described herein.

FIG. 56 depicts a chart showing settling velocities observed for different OD fractions in suspension samples of L. reuteri LAB1 at different temperature and pH. Their allocation to different pH categories for further analysis is provided in the pH-sim row.

FIG. 57 depicts a chart showing settling velocities observed for different OD fractions in suspension samples of L. reuteri CMP19 at different temperature and pH. Their allocation to different pH categories for further analysis is provided in the pH-sim row.

FIG. 58 depicts a chart showing settling velocities observed for different OD fractions in suspension samples of L. reuteri CMP21 at different temperature and pH. Their allocation to different pH categories for further analysis is provided in the pH-sim row.

FIG. 59 depicts a chart showing settling velocities observed for different OD fractions in suspension samples of three-strain Lactobacillus reuteri-containing Consortium-S(ConS) at different temperature and pH. Their allocation to different pH categories for further analysis is provided in the pH-sim row.

FIG. 60 depicts a chart showing viable cell counts (MPN/mL) of Lactobacillus reuteri LAB1 in 2.5 mM equimolar phosphate buffer over time incubated at different temperatures as derived from MPN analysis and associated quality parameters of the analysis according to Jarvis et al. (Journal of Applied Microbiology 109: 1660-1667, 2010). CI=Confidence interval. SD=standard deviation. MPN values of 0.0E+00 MPN/mL indicate that no viable cells were detected within the LOD of the applied method.

FIG. 61 depicts a chart showing viable cell counts (MPN/mL) of Lactobacillus reuteri CMP19 in 2.5 mM equimolar phosphate buffer over time incubated at different temperatures as derived from MPN analysis and associated quality parameters of the analysis according to Jarvis et al. (Journal of Applied Microbiology 109: 1660-1667, 2010). CI=Confidence interval. SD=standard deviation. MPN values of 0.0E+00 MPN/mL indicate that no viable cells were detected within the LOD of the applied method.

FIG. 62 depicts a chart showing viable cell counts (MPN/mL) of Lactobacillus reuteri CMP21 in 2.5 mM equimolar phosphate buffer over time incubated at different temperatures as derived from MPN analysis and associated quality parameters of the analysis according to Jarvis et al. (Journal of Applied Microbiology 109: 1660-1667, 2010). CI=Confidence interval. SD=standard deviation. MPN values of 0.0E+00 MPN/mL indicate that no viable cells were detected within the LOD of the applied method.

FIG. 63 depicts a chart showing viable cell counts (MPN/mL) of three-strain Lactobacillus reuteri consortium ConS in 2.5 mM equimolar phosphate buffer over time incubated at different temperatures as derived from MPN analysis and associated quality parameters of the analysis according to Jarvis et al. (Journal of Applied Microbiology 109: 1660-1667, 2010). CI=Confidence interval. SD=standard deviation. MPN values of 0.0E+00 MPN/mL indicate that no viable cells were detected within the LOD of the applied method.

### DETAILED DESCRIPTION

Provided herein, inter alia, are compositions, methods and kits for optimally delivering viable microbial cells (e.g. probiotics or “direct fed microbials” (DFMs)) to livestock or other living organisms (e.g. plants) via a water line. Ideally, optimization of delivery of viable microbial cells 1) provides high stability (i.e., maintenance of viability) upon release into a rehydration liquid and then into a water line; 2) provides fast dissolution and dispersion in a rehydration liquid (such as water); 3) ensures the cells are suspended and remain in suspension while exhibiting reduced settling after being pumped into a water line; and 4) provide high stability (i.e. maintenance of viability) in the waterline.

To this end, the inventors of the instant application have surprisingly discovered that suspending microbial cells in a buffered solution with increased pH and optionally a thickening agent slows settling velocity without inhibiting the viability of microorganisms in water lines. As will be discussed in the Examples section, this was demonstrated using a broad range of microorganisms including gram-positive and gram-negative bacteria, as well as fungi. In addition, without being bound to theory, it is believed that the pH for the slowest settling is found on the point with the lowest negative Zeta potential for a given organism. As shown in Example 1, microbial cells settle in aqueous solutions with little to no aggregation prior to settling. Therefore, use of the DFM/microbial water line compositions having increased pH disclosed herein results in reduced settling velocities of the DFMs/microbial cells during transit through the water line which directly translates into more microbial cells predictably and reproducibly being delivered to livestock or other living organisms.

### I. Definitions

As used herein, the phrase “a buffer sufficient to maintain pH of water at about or greater than about 6.5” refers to any compound with at least one functional group with a hydrogen dissociation constant (pKa) of at least about 5.0 (such as any of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0 or higher) that can be used as buffer.

A “buffer” as used herein is an agent that maintains a stable pH in a solution within a specific pH range. Buffering ranges are determined by pKa.

The term “carboxylic acid” as used herein refers to a compound with a —C(O)OH group.

The term “amine” as used herein refers to primary, secondary, and tertiary amines having, e.g., the formula N(group)₃ wherein each group is independently H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “amide” as used herein, represents a group of formula or “—C(O)N(R^{x})(R^{y})” or —“NR^{x}C(O)R^{y}” wherein R^{x} and R^{y} can be the same or independently H, alkyl, aryl, cycloalkyl, cycloalkenyl, heterocycle.

The term “imine” as used herein, refers to the radical ═NH.

The term “hydroxy,” as used herein, alone or in combination, refers to —OH.

The term “sulfonic,” as used herein, refers to —SO₃H.

As used herein, “microorganism” or “microbe” refers to a bacterium, a fungus, a virus, a protozoan, and other microbes or microscopic organisms.

As used herein the term “direct fed microbial” is used interchangeably with “probiotic” or “beneficial microbe” or “microbe” and refers to a composition for consumption by animals (i.e. as an or as a component of animal feed) or other organisms (e.g. plants) that contains viable microorganisms, i.e. microorganisms that are capable of living and reproducing and which provides one or more benefits to the animal or other organism (for example, improved gut health or resistance to disease). See, for example, U.S. Pat. No. 8,420,074. A direct fed microbial may comprise one or more (such as any of 1, 2, 3, 4, 5, or 6 or more) of any microbial strains.

A microbial “strain” as used herein refers to a microbe (e.g., a bacterium, yeast, or fungus) which remains genetically unchanged when grown or multiplied (e.g. by clonal expansion). The multiplicity of identical bacteria is included.

By “at least one strain,” is meant a single strain but also mixtures of strains comprising at least two strains of microorganisms. By “a mixture of at least two strains,” is meant a mixture of two, three, four, five, six or even more strains. In some embodiments of a mixture of strains, the proportions can vary from 1% to 99%. When a mixture comprises more than two strains, the strains can be present in substantially equal proportions in the mixture or in different proportions.

For purposes of this disclosure, a “biologically pure strain” means a strain containing no other bacterial strains in quantities sufficient to interfere with replication of the strain or to be detectable by normal bacteriological techniques. “Isolated,” when used in connection with the organisms and cultures described herein, includes not only a biologically pure strain, but also any culture of organisms which is grown or maintained other than as it is found in nature. It will also be clear that addition of other microbial strains, carriers, additives, enzymes, yeast, or the like will also provide one or more benefits or improvement of one or more metrics in an animal and will not constitute a substantially different DFM.

The term “thickening agent,” as used herein, refers to any of a variety of generally hydrophilic materials which, when incorporated in the present compositions, may act as viscosity modifying agents, emulsifying agents, gelling agents, suspending agents, and/or stabilizing agents.

The term “water line” as used herein means any channel, such as a watering canal, vats or line, such as plastic or metal tubes, that are suitable for holding and transporting water, for use in connection with the daily operation of a livestock growing facility or a crop-growing facility, for example a farm.

The term “pumping means” as used herein means a pump or other means (such as a dosimeter or driving force, e.g. resulting from gravity) of transporting water or water compositions into and/or through the water line.

As used herein, “nipple drinkers” refer to any device or means that permits livestock to access the water transported by a water line. In some embodiments, the livestock can activate water flow through the nipple drinker by pecking at, biting, or sucking on the nipple drinker.

The term, “drip irrigation” as used herein and in the claims includes microsprinklers, drip and subsurface drip systems typically used to provide moisture to plants in agricultural settings.

As used herein, “subject” includes any living organism that requires water to live. Non-limiting examples of subjects can include mammals (for example, humans, non-human primates, and livestock) and plants (for example, crop plants, such as, without limitation, soy, cotton, canola, maize, wheat, sunflower, sorghum, alfalfa, barley, millet, rice, tobacco, fruit and vegetable crops, cannabis, and turf grass).

As used herein, “livestock” includes any animal kept for commercial useful purposes, such as domesticated animals that are raised for producing commodities such as food (e.g., milk and meat), animal products (e.g., fiber), or working in an agricultural or other commercial activity. Livestock includes, without limitation, swine (e.g., pigs, boars, sows, growers, and finishers), ruminants, horses, and poultry.

The term “poultry,” as used herein, means domesticated birds kept by humans for their eggs, their meat or their feathers. These birds are most typically members of the superorder Galloanserae, especially the order Galliformes which includes, without limitation, chickens (such as layers or broilers), quails, ducks, geese, emus, ostriches, pheasant, and turkeys.

“Ruminants” generally refer to even-toed hoofed mammals that chew the cud and have a complex multi-chambered stomach. Animals that would be classified as ruminants include cattle, sheep, goats, deer, llamas, antelope, and others. Because of having multiple stomachs, the digestive system and process of ruminants differs substantially from that of monogastric animals.

As used herein “administer” or “administering” is meant the action of introducing one or more of the buffered DFM-containing water line compositions disclosed herein to livestock or animals via a water line.

The term “zeta potential” as used herein refers to the electrical potential at the slipping plane. The “slipping plane” refers to the interface which separates a mobile fluid from a fluid that remains attached to a surface. The zeta potential is caused by the net electrical charge contained within the region bounded by the slipping plane, and also depends on the location of that plane. Thus, it is widely used for quantification of the magnitude of the charge. Moreover, zeta potential is an important and readily measurable indicator of the stability of colloidal dispersions. The magnitude of the zeta potential indicates the degree of electrostatic repulsion between adjacent, similarly charged particles in a dispersion. For molecules and particles that are small enough, a high zeta potential will confer stability, i.e., the solution or dispersion will resist aggregation. When the potential is small, attractive forces may exceed this repulsion and the dispersion may break and flocculate. So, colloids with high zeta potential (negative or positive) are electrically stabilized while colloids with low zeta potentials tend to coagulate or flocculate. More detailed information on zeta potential and its application to bacterial systems is provided by Ferreyra Maillard et al. (BBA—Biomembranes 1863: 183591 (2021)) and Hanaor et al. (Journal of the European Ceramic Society. 32 (1): 235-244), the disclosures of which are incorporated by reference herein in their entireties).

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interfere with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

Provided herein are compositions suitable for water line administration of direct fed microbials. These compositions contain a buffer sufficient to maintain pH of water at about or greater than about 6.5.

### A. Buffers

Provided herein are water line compositions comprising a buffer sufficient to maintain pH of water at about or greater than about 6.5; and one or more direct fed microbials (DFMs). Any buffer capable of maintaining the pH of water or a water-based solution at about or greater than 6.0 (such as any of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, or greater, so long as DFM viability is not significantly affected by increasingly basic pH).

In some embodiments, the buffer can act as a biological buffer, as, e.g. described by Good & Izawa (Methods Enzymol, 1972. 24: p. 53-68) and Good et al. (Biochemistry, 1966. 5(2). p. 467-77), incorporated by reference herein. These buffers can include, but are not limited to, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and/or MES (2-(N-morpholino)ethanesulfonic acid.

In further embodiments, the buffer is compatible with safe human and animal consumption, and/or has GRAS (generally recognized as save) status, and/or is approved by regulatory agencies for their use in feed or food applications.

The buffers for use in the water line compositions and methods disclosed are suitably water soluble.

In some instances, the buffer can be either derived or produced from biological material. In one embodiment, the buffer contains at least one carboxylic acid group. In yet another embodiment of the invention, the buffer has at least one amine group, at least one imine group, at least one amide group, or at least one other nitrogen containing functional group that can accept a proton. In yet another embodiment, the buffer has at least one hydroxy group. In yet another embodiment, the buffer has at least one sulfonic and/or at least one phosphonic group.

Examples of buffers derived or produced from biological materials include, without limitation, nucleobases (e.g. uracil, thymine, cytosine, guanine, adenine), nucleosides and nucleotides and their derivatives, amino acids and their derivatives, comprising but not limited to alpha-alanine, beta-alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and aminolevulinic acid, as well as organic acids and their derivatives, comprising but not limited to maleic acid, malonic acid, oxaloacetic acid, methylmalonic acid, succinic acid, malic acid, itaconic acid, glutaric acid, methylsuccinic acid, pivalic acid, ascorbic acid, citric acid, isocitric acid, adipic acid, salicylic acid, protocatechuic acid, pimelic acid, phthalic acid and suberic acid.

An organic buffer compounds can also be used in the water line compositions and methods disclosed herein. These can include, without limitation, carbonic acid, phosphoric acid, sulfurous acid (H₂SO₃) and ammonia.

In some embodiments, the water line compositions and methods disclosed herein comprise a single buffer. In other embodiments, the water line compositions and methods disclosed herein comprise a mixture of buffers (such as a mixture of 2, 3, 4, 5, 6, 7, 8, 9, or more buffers, such as pure buffer compounds) or are undefined (e.g. starting from complex mixtures, comprising but not limited to fruit juices, vegetable juices or other plant extracts).

It is understood that counter-ion of the buffer for use in the water line compositions and methods disclosed herein in deprotonated form can be organic and/or inorganic. In one preferable embodiment, the counter-ion comprises at least one element of the alkali metal group (e.g. Li, Na, K, Rb, Cs, Fr; for example, K₂HPO₄, KH₂PO₄, K₂HPO₄/KH₂PO₄, Na₂CO₃, NaHCO₃, or Na₂CO₃/NaHCO₃). In another embodiment, the counter-ion comprises at least one element of the alkali earth metals group (e.g. Be, Mg, Ca, Sr, Ba, Ra). In yet another embodiment, the counter-ion comprises at least one element of the transition metals (e.g. Mn, Fe, Cu, Zn, etc.). Other anionic counter ions can include sulfur, phosphorus, selenium, carbon, nitrogen, silicia and their oxidated derivatives (e.g. sulfate, phosphate, selenate, carbonate, nitrate, silicate, etc.).

In some embodiments, the buffer sufficient to maintain pH of water at about or greater than about 6.5 is at a concentration of about 1-2000 mM, such as any of about 0.001-20 mM, 0.01-20 mM, 0.1-20 mM, 0.1-15 mM, 0.1-10 mM, 0.1-5 mM, 0.5-5 mM, 0.75-4.5 mM, 1-4 mM, 1.5-3.5 mM, 2-3 mM, 50-1000 mM, 100-750 mM, 150-500 mM, 200-400 mM, 250-350 mM, 275-325 mM, 250-1750 mM, 500-1500 mM, 750-1250 mM or any of about 0.001 mM, 0.005 mM, 0.01 mM, 0.015 mM, 0.05 mM, 0.075 mM, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, 300 mM, 301 mM, 302 mM, 303 mM, 304 mM, 306 mM, 307 mM, 308 mM, 309 mM, 310 mM, 311 mM, 312 mM, 313 mM, 314 mM, 315 mM, 316 mM, 317 mM, 318 mM, 319 mM, 320 mM, 321 mM, 322 mM, 323 mM, 324 mM, 325 mM, 326 mM, 327 mM, 332 mM, 329 mM, 330 mM, 331 mM, 332 mM, 333 mM, 334 mM, 335 mM, 336 mM, 337 mM, 338 mM, 339 mM, 340 mM, 341 mM, 342 mM, 343 mM, 344 mM, 345 mM, 346 mM, 347 mM, 348 mM, 349 mM, 350 mM, 355 mM, 360 mM, 365 mM, 370 mM, 375 mM, 380 mM, 385 mM, 390 mM, 395 mM, 400 mM, 405 mM, 410 mM, 415 mM, 420 mM, 425 mM, 430 mM, 435 mM, 440 mM, 445 mM, 450 mM, 455 mM, 460 mM, 465 mM, 470 mM, 475 mM, 480 mM, 485 mM, 490 mM, 495 mM, 500 mM, 510 mM, 520 mM, 530 mM, 540 mM, 550 mM, 560 mM, 570 mM, 580 mM, 590 mM, 600 mM, 610 mM, 620 mM, 630 mM, 640 mM, 650 mM, 660 mM, 670 mM, 680 mM, 690 mM, 700 mM, 725 mM, 750 mM, 775 mM, 800 mM, 825 mM, 850 mM, 875 mM, 900 mM, 925 mM, 950 mM, 975 mM, 1000 mM, 1100 mM, 1200 mM, 1300 mM, 1400 mM, 1500 mM, 1600 mM, 1700 mM, 1800 mM, 1900 mM, 2000 mM, or more, inclusive of all concentrations falling in between these values. In some embodiments, the concentration of the buffer is the concentration of the buffer in a stock solution. In other embodiments, the concentration of the buffer is the concentration of the buffer in a water line.

In yet other embodiments, the water line compositions comprising a buffer sufficient to maintain pH of water at about or greater than about 6.5 and one or more direct fed microbials (DFMs) disclosed herein can be maintained at a temperature of less than about 22° C., such as any of about 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., or 1° C.

### B. Direct Fed Microbials

Direct fed microbials (DFMs) refer to the feeding of beneficial microbes to animals or other organisms, such as domestic birds or plants (such as crops), when they are under periods of stress (disease, ration changes, environmental or production challenges) or as a part of a daily nutritional regimen to prevent disease and facilitate nutrient usage during digestion. Probiotics is another term for this category of additives (for example plant or feed additives). Probiotics or DFMs have been shown to improve animal or plant performance in controlled studies. In some embodiments, DFMs include both direct fed bacteria and/or yeast-based products and, in particular embodiments, include viable microorganisms. The term “viable microorganism” means a microorganism which is metabolically active or able to differentiate.

In some embodiments, the DFM can include a bacteria (a gram positive or gram negative bacteria), algae, or fungi (such as a filamentous fungi or a yeast). In one embodiment, the DFM may be a spore forming bacterium and hence the term DFM may refer to a composition that is comprised of or contains spores, e.g., bacterial spores. Therefore, in one embodiment the term “viable microorganism” as used herein may include microbial spores, such as endospores or conidia. In another embodiment, the DFM in the feed additive composition according to the present invention is not comprised of or does not contain microbial spores, e.g. endospores or conidia (i.e., the DFM is non-spore forming). The microorganism may be a naturally-occurring microorganism or it may be a transformed microorganism.

Preferably, the DFM described herein comprises microorganisms which are generally recognized as safe (GRAS) and, preferably are GRAS-approved. A person of ordinary skill in the art will readily be aware of specific species and/or strains of microorganisms from within the genera described herein which are used in the food and/or agricultural industries and which are generally considered suitable for animal consumption.

The DFM described herein may decrease or prevent intestinal establishment of pathogenic microorganism (such as Clostridium perfringens and/or E. coli and/or Salmonella spp and/or Campylobacter spp.). In other words, the DFM may be antipathogenic. The term “antipathogenic” as used herein means the DFM counters an effect (negative effect) of a pathogen.

### 1. Genus/Species

A DFM for inclusion in the water line compositions or methods or kits disclosed herein may comprise microorganisms from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium or Megasphaera and combinations thereof.

In another aspect, the DFM may be one or more of the following Bacillus spp.: B. subtilis, B. licheniformis, B. lentus, B. brevis, B. stearothermophilus, Bacillus cereus, B. alkalophilus, B. amyloliquefaciens, B. licheniformis, B. clausii, B. halodurans, B. megaterium, B. coagulans, B. circulans, B. gibsonii, B. pumilis, B. velezensis, or B. thuringiensis, and combinations of any thereof (including combinations of DFMs from within this genus or combinations with additional genera and/or species disclosed herein). The genus “Bacillus”, as used herein, includes all species within the genus “Bacillus,” as known to those of skill in the art. It is recognized that the genus Bacillus continues to undergo taxonomical reorganization. Thus, it is intended that the genus include species that have been reclassified, including but not limited to such organisms as Bacillus stearothermophilus, which is now named “Geobacillus stearothermophilus”, or Bacillus polymyxa, which is now “Paenibacillus polymyxa.” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In a further aspect, the DFM may be one or more of the following Lactococcus spp: Lactococcus cremoris, L. chungangensis, L. formosensis, L. fujiensis, L. garvieae, L. hircilactis, L. lactis, L. laudensis, L. nasutitermitis, L. piscium, L. plantarum, L. raffinolactis, L. taiwanensis or Lactococcus lactis and combinations thereof (including combinations of DFMs from within this genus or combinations with additional genera and/or species disclosed herein). The genus “Lactococcus”, as used herein, includes all species within the genus “Lactococcus,” as known to those of skill in the art.

The DFM can further be one or more of the following Lactobacillus spp: Lactobacillus buchneri, Lactobacillus acidophilus, Lactobacillus casei, Lactobacillus kefiri, Lactobacillus bifidus, Lactobacillus brevis, Lactobacillus heliveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus farciminis, Lactobacillus rhamnosus, Lactobacillus crispatus, Lactobacillus gasseri, Lactobacillus johnsonii or Lactobacillus jensenii, and combinations of any thereof (including combinations of DFMs from within this genus or combinations with additional genera and/or species disclosed herein). The genus “Lactobacillus”, as used herein, includes all species within the genus “Lactobacillus,” as known to those of skill in the art. It is recognized that the genus Lactobacillus continues to undergo taxonomical reorganization. For example, as of March 2020, Lactobacilli comprised 261 species that are extremely diverse phenotypically, ecologically, and genotypically. Given recent advances in whole genome sequencing and comparative genomics, the genus Lactobacillus was recently divided into 25 separate genera with strains belonging to previously designated Lactobacilli species being transferred to new species and/or genera (see Zheng et al., 2020, Int. J. Syst. Evol. Microbiol., 70.2782-2858; Pot et al., Trends in Food Science & Technology 94 (2019) 105-113; and Koutsoumanis et al., 2020, EFSA Journal, 18(7):6174, the disclosures of each of which are incorporated by reference herein). For purposes of the instant disclosure, the previous classification of Lactobacillus species will continue to be employed. However, in some embodiments Lactobacillus agilis is also classified as as Ligilactobacillus agilis. In other embodiments, Lactobacillus salivarius is also classified as Ligilactobacillus salivarius. In further embodiments, Lactobacillus reuteri is also classified as Limosilactobacillus reuteri.

In yet another aspect, the DFM may be one or more of the following Bifidobacteria spp: Bifidobacterium lactis, Bifidobacterium bifidium, Bifidobacterium longum, Bifidobacterium animalis (including Bifidobacterium animalis subspecies animalis), Bifidobacterium breve, Bifidobacterium infantis, Bifidobacterium catemulatum, Bifidobacterium pseudocatemilatum, Bifidobacterium adolescentis, or Bifidobacterium angulatum, and combinations of any thereof (including combinations of DFMs from within this genus or combinations with additional genera and/or species disclosed herein). The genus “Bifidobacterium”, as used herein, includes all species within the genus “Bifidobacterium,” as known to those of skill in the art.

In another aspect, the DFM may be one or more of the following Enterococcus spp: E. alcedinis, E. aquimarinus, E. asini, E avium, E. bulliens, E. burkinafasonensis, E. caccae, E. camelliae, E. canintestini, E. canis, E. casseliflavus, E. cecorum, E. columbae, E. crotali, E. devriesei, E. diestrammenae, E. dispar, E. durans, E. eurekensis, E. faecalis, E. faecium, E. gallinarum, E. gilvus, E. haemoperoxidus, E. hermanniensis, E. hirae, E. hulanensis, E. italicus, E. lactis, E. lemanii, E. malodoratus, E. massiliensis, E. mediterraneensis, E. moraviensis, E. mundtii, E. olivae, E. pallens, E. phoeniculicola, E. plantarum, E. pseudoavium, E. quebecensis, E. raffinosus, E. ratti, E. rivorum, E. rotai, E. saccharolyticus, E. saigonensis, E. silesiacus, E. sulfureus, E. solitarius, E. songbeiensis, E. termitis, E. thailandicus, E. ureasiticus, E. ureilyticus, E. viikkiensis, E. villorum, E. wangshanyuanii, E. xiangfangensis, or E. xinjiangensis, and combinations of any thereof (including combinations of DFMs from within this genus or combinations with additional genera and/or species disclosed herein). The genus “Enterococcus”, as used herein, includes all species within the genus “Enterococcus,” as known to those of skill in the art.

The DFM can further be one or more of the following Megasphaera spp: Megasphaera hominis, Megasphaera cerevisiae, Megasphaera elsdenii, Megasphaera micronuciformis, egasphaera paucivorans, or Megasphaera sueciensis, and combinations of any thereof (including combinations of DFMs from within this genus or combinations with additional genera and/or species disclosed herein). The genus “Megasphaera”, as used herein, includes all species within the genus “Megasphaera,” as known to those of skill in the art. In one non-limiting embodiment, the Megasphaera spp. (e.g. M. elsdenii) can be combined with a yeast, such as any of the yeasts described below.

In a further aspect, the DFM may be one or more of the following yeast species from Saccharomyces: Saccharomyces arboricolus, Saccharomyces bayanus, Saccharomyces bulderi, Saccharomyces cariocanus, Saccharomyces cariocus, Saccharomyces cerevisiae, Saccharomyces cerevisiae var. boulardii, Saccharomyces chevalieri, Saccharomyces dairenensis, Saccharomyces ellipsoideus, Saccharomyces eubavanus, Saccharomyces exiguous, Saccharomyces florentinus, Saccharomyces fragilis, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, or Saccharomyces zonatus, and combinations of any thereof (including combinations of DFMs from within this genus or combinations with additional genera and/or species disclosed herein). The genus “Saccharomyces”, as used herein, includes all species within the genus “Saccharomyces,” as known to those of skill in the art. In one non-limiting embodiment, the Megasphaera spp. (e.g. M. elsdenii) can be combined with a yeast, such as any of the yeasts described herein.

In yet another aspect, the DFM may be one or more of the following yeast species: Pichia kudriavzevii, Candida krusei, Saccharomyces krusei, Endomyces krusei, Monilia krusei, Candida krusei, Myceloblastanon krusei, Geotrichoides krusei, Trichosporon krusei, Mycoloruloides krusei, Enantiothammus braulti, Blastodendrion braulti, Monilia parakrusei, Myceloblastanon parakrusei, Castellania parakrusei, Candida parakrusei, Mycoderma chevalieri, Candida chevalieri, Mycoderma monosa, Mycoderma bordetii, Monilia inexpectata, Mycocandida inexpectata, Pseudomonilia inexpectata, Trichosporon dendriticum, Candida dendritica, Castellania africana, Castellania halcanica, Monilia krusoides, Pseudomycoderma miso, Candida castellanii, Candida tamarindi, Procandida tamarindii, Issatchenkia orientalis, Candida lobate, Endoblastomyces thermophilus, Candida requinyii, Candida soosii, Pichia orientalis, Candida acidothermophilum, Candida brassicae, Candida ethanothermophilum, Candida melinii, Candida hinoensis, or Candida solicola. In one non-limiting embodiment, the Megasphaera spp. (e.g. M. elsdenii) can be combined with a yeast, such as any of the yeasts described herein.

In a further aspect, the DFM may be one or more of the following yeast species from Schizosaccharomyces: S. cryophilus, S. japonicus, S. oclosporus, or S. pombe, and combinations of any thereof (including combinations of DFMs from within this genus or combinations with additional genera and/or species disclosed herein). The genus “Schizosaccharomyces”, as used herein, includes all species within the genus “Schizosaccharomyces,” as known to those of skill in the art.

In another aspect, the DFM may be one or more of the following species: Pediococcus spp. (including all species within the genus Pediococcus), Pediococsus acidilactici, Propionibacterium spp. including all species within the genus Propionibacterium), Propionibaclerium thoenii, Clostridium spp. (including all species within the genus Clostridium), Clostridium butyricum, and combinations thereof.

A direct-fed microbial described herein comprising one or more bacterial strains may be of the same type (genus, species and strain) or may comprise a mixture of genera, species and/or strains.

### 2. Strains

Alternatively, the DFM for inclusion in the water line compositions or methods or kits disclosed herein can be one or more of the product strains or microorganism strains contained in those products disclosed in International Patent Application Publication No. WO2012110778 (incorporated by reference herein), and summarized as follows: Bacillus subtilis strain 2084 Accession No. NRRLB-50013, Bacillus subtilis strain LSSAO1 Accession No. NRRL B-50104, and Bacillus subtilis strain 15A-P4 ATCC Accession No. PTA-6507 (from Enviva Pro® (formerly known as Avicorr®); Bacillus subtilis Strain C3102 (from Calsporin®); Bacillus subtilis Strain PB6 (from Clostat®); Bacillus pumilis (8G-134); Enterococcus NCIMB 10415 (SF68) (from Cylactin®); Bacillus subtilis Strain C3102 (from Gallipro® & GalliproMax®); Bacillus licheniformis (from Gallipro®Tect®); Enterococcus and Pediococcus (from Poultry Star®); Lactobacillus, Bifidobacterium and/or Enterococcus from Protexin®); Bacillus subtilis strain QST 713 (from Proflora®); Bacillus amyloliquefaciens CECT-5940 (from Ecobiol® & Ecobiol® Plus); Enterococcus faecium SF68 (from Fortiflora®); Bacillus subtilis and Bacillus licheniformis (from BioPlus2B®); Lactic acid bacteria 7 Enterococcus faecium (from Lactiferm®); Bacillus strain (from CSI®); Saccharomyces cerevisiae (from Yea-Sacc®); Enterococcus (from Biomin IMB52®); Pediococcus acidilactici, Enterococcus, Bifidobacterium animalis ssp. animalis, Laclobacillus reuteri, Lactobacillus salivarius ssp. salivarius (from Biomin C5®); Lactobacillus farciminis (from Biacton®); Enterococcus (from Oralin E1707®); Enterococcus (2 strains), Lactococcus lactis DSM 1103 (from Probios-pioneer PDFM®); Lactobacillus rhamnosus and Lactobacillus farciminis (from Sorbiflore®); Bacillus subtilis (from Animavit®); Enterococcus (from Bonvital®); Saccharomyces cerevisiae (from Levucell SB 20®); Saccharomyces cerevisiae (from Levucell SC 0 & SC10® ME); Pediococcus acidilacti (from Bactocell), Saccharomyces cerevisiae (from ActiSaf® (formerly BioSaf®)); Saccharomyces cerevisiae NCYC Sc47 (from Actisaf® SC47); Clostridium butyricum (from Miya-Gold®); Enterococcus (from Fecinor and Fecinor Plus®); Saccharomyces cerevisiae NCYC R-625 (from InteSwine®); Saccharomyces cerevisia (from BioSprint®); Enterococcus and Lactobacillus rhamnosus (from Provita®); Bacillus subtilis and Aspergillus oryzae (from PepSoyGen-C®); Bacillus cereus (from Toyocerin®); Bacillus cereus var. toyoi NCIMB 40112/CNCM I-1012 (from TOYOCERIN®), or other DFMs such as Bacillus licheniformis and Bacillus subtilis (from BioPlus® YC) and Bacillus subtilis (from GalliPro®).

The DFM can be Enviva® PRO, which is commercially available from Danisco A/S. Enviva Pro® is a combination of Bacillus strain 2084 Accession No. NRRL B-50013, Bacillus strain LSSAO1 Accession No. NRRL B-50104 and Bacillus strain 15A-P4 ATCC Accession No. PTA-6507 (as taught in U.S. Pat. No. 7,754,469 B—incorporated herein by reference).

Additional strains for inclusion in the water line compositions or methods or kits disclosed herein include Lactobacillus reuteri strain S1, L. reuteri strain S2, L. reuteri strain S3, L. gallinarum strain H1, L. salivarius strain H2, L. agilis strain H3, L. salivarius strain A1, L. reuteri strain A2, L. reuteri strain A3, L. agilis strain D1, L. salivarius strain D2, and L. crispatus strain D3, which are also referred to herein as S1, S2, S3, H1, H2, H3, A1, A2, A3, D1, D2, and D3, respectively. Additional information regarding these strains can be found in International Patent Application Publication No. WO2021034660, the disclosure of which is incorporated by reference herein.

L. reuteri strain S1, L. reuteri strain S2, L. reuteri strain S3, L. reuteri strain A2, L. gallinarum strain H1, L. salivarius strain H2, and L. agilis strain H3 were deposited on Jul. 24, 2019 at the Westerdijk Fungal Biodiversity Institute (WFDB), Uppsalalaan 8, 3584 CT, Utrecht, The Netherlands and given accession numbers CBS 145921, CBS 145922, CBS 145923, CBS 145924, CBS145918, CBS145919, and CBS 145920, respectively. The deposits were made under the provisions of the Budapest Treaty on the International Recognition of the Deposit of Microorganisms for the Purposes of Patent Procedure. One or more strain provided herein can be used as a direct-fed microbial (DFM).

Additional strains for inclusion in the water line compositions or methods or kits disclosed herein include Lactobacillus reuteri strain S1a, L. reuteri strain S1b, L. reuteri strain S2a, and L. reuteri strain S2b, which are also referred to herein as S1a, S1b, S2a, and S2b, respectively. These strains are derived from L. reuteri strains S1 and S2. Genome analysis of S1 and S2 revealed that these strains contained a number of antibiotic resistance markers (AMRs). As AMRs have been implicated in the spread of antibiotic resistance in animal and humans, these AMRs were removed from the genomes of strains S1 and S2. The new strains, now engineered (i.e. are non-naturally occurring) to lack one or more AMRs, were designated L. reuteri strain S1a, L. reuteri strain S1b, L. reuteri strain S2a, and L. reuteri strain S2b. Additional information regarding these strains can be found in U.S. Patent Application Publication No. 2021/0153521, the disclosure of which is incorporated by reference herein.

L. reuteri strain S1a (ABM01), L. reuteri strain S1b (ABM02), L. reuteri strain S2a (ABM03), and L. reuteri strain S2b (ABM04) were deposited on Dec. 2, 2020 at the Westerdijk Fungal Biodiversity Institute (WFDB), Uppsalalaan 8, 3584 CT, Utrecht, The Netherlands and given accession numbers CBS 147267, CBS 147268, CBS 147269, and CBS 147270, respectively. The deposits were made under the provisions of the Budapest Treaty on the International Recognition of the Deposit of Microorganisms for the Purposes of Patent Procedure. One or more strain provided herein can be used as a direct-fed microbial (DFM).

Further strains for inclusion in the water line compositions or methods or kits disclosed herein include Anaerotruncus colihominis strain W1, Anaerolruncus colihominis strain W2, Anaerotruncus colihominis strain W3, Anaerotruncus colihominis strain W4, Coprococcus sp. strain M1, Anaerotruncus colihominis strain M2, Clostridium lactatifermentans strain M3, Pseudoflavonifractor capillosus strain M4, Clostridium lactatifermentans strain 2F1, Lactobacillus salivarius strain 2F2, and Lactobacillus reuteri strain 2F3 which are also referred to herein as W1, W2, W3, W4, M1, M2, M3, M4, 2F1, 2F2, and 2F3, respectively. Additional information regarding these strains can be found in International Patent Application Publication No. WO2021080864, the disclosure of which is incorporated by reference herein.

Anaerotruncus colihominis strain W1, Anaerotruncus colihominis strain W2, Anaerotruncus colihominis strain W3, and Anaerotruncus colihominis strain W4 were deposited on Oct. 9, 2019 at the Westerdijk Fungal Biodiversity Institute (WFDB), Uppsalalaan 8, 3584 CT, Utrecht, The Netherlands and given accession numbers CBS 146120, CBS 146122, CBS 146123, and CBS 146121, respectively. The deposits were made under the provisions of the Budapest Treaty on the International Recognition of the Deposit of Microorganisms for the Purposes of Patent Procedure. One or more strain provided herein can be used as a direct-fed microbial (DFM).

Yet further strains provided herein for inclusion in the water line compositions or methods or kits disclosed herein include oxygen-tolerant strains of Megasphaera elsdenii which include M. elsdenii ACD1265, M. elsdenii ACD1096-A01, M. elsdenii ACD1096-B01, M. elsdenii ACD1096-E01, M. elsdenii ACD1096-C02, M. elsdenii ACD1096-C05, M. elsdenii ACD1096-H05, M. elsdenii ACD1096-B03, AM. elsdenii ACD1141-C10, M. elsdenii ACD1141-D10, M. elsdenii ACD 1141, M. elsdenii ACD 1141E, M. elsdenii ACD 1141F, M. elsdenii ACD1265E, and M. elsdenii ACD1265F which are also referred to herein as ACD1265, ACD1096-A01, ACD1096-B01, ACD1096-E01, ACD1096-C02, ACD1096-C05, ACD1096-H05, ACD1096-B03, ACD1141-C10, and ACD1141-D10, ACD1141, ACD1141E, ACD1141F, ACD1265E, and ACD1265F, respectively. Additional information regarding these strains can be found in International Patent Application Publication No. 2021158927, the disclosure of which is incorporated by reference herein.

A M. elsdenii ACD1265, M. elsdenii ACD1141, M. elsdenii ACD1141E, M. elsdenii ACD1141F, M. elsdenii ACD1265E, and M. elsdenii ACD1265F were deposited on Dec. 18, 2019 at the Westerdijk Fungal Biodiversity Institute (WFDI), Uppsalalaan 8, 3584 CT, Utrecht, The Netherlands and given accession numbers CBS 146328, CBS 146325, CBS 146326, CBS 146327, CBS 146329, and CBS 146330, respectively. The deposits were made under the provisions of the Budapest Treaty on the International Recognition of the Deposit of Microorganisms for the Purposes of Patent Procedure. One or more strain provided herein can be used as a direct-fed microbial (DFM).

In some embodiments, additional M. elsdenii cells suitable for use in the compositions or methods, or kits disclosed herein are from a strain having a deposit number selected from the group consisting of: ATCC® 25940, ATCC® 17752, ATCC® 17753, NCIMB 702261, NCIMB 702262, NCIMB 702264, NCIMB 702331, NCIMB 702409, NCIMB 702410, NCIMB 41787, NCIMB 41788, NRRL 18624, NIAH 1 102, and a biologically pure bacterial culture of M. elsdenii having substantially the same 16S ribosomal RNA sequence as that of the M. elsdenii strain deposited on Mar. 18, 2002 at NCIMB, Aberdeen, Scotland, UK under number NCIMB 41125.

Commercially available yeast strains for use in any of the compositions or methods or kits disclosed herein can include, without limitation, Ethanol red (LeSaffre), Zenith thermostable yeast or Zenith yeast concentrate (FLEISCHMANNS YEAST (AB Mauri)), Saf-instant or Saf-instant Gold (LeSaffre), Fleischmann's Instant Dry Yeast (FLEISCHMANNS YEAST (AB Mauri)), Red Star (LeSaffre), Instant Yeast HS 2141 or Instant Yeast 2174 ((FLEISCHMANNS YEAST (AB Mauri)), or Summit Ethanol dry yeast 6007 (AB Mauri). In one non-limiting embodiment, a Megasphaera spp. (e.g. M. elsdenii) can be combined with a yeast, such as any of the yeasts described below.

Further strains for inclusion in the water line compositions or methods or kits disclosed herein include Bifidobacterium animalis subsp. lactis strain B1-04 and/or Lactobacillus acidophilus strain NCFM. These bacterial strains were deposited by DuPont Nutrition Biosciences ApS, of Langebrogade 1, DK-1411 Copenhagen K, Denmark, in accordance with the Budapest Treaty at the Leibniz-Institut Deutsche Sammlung von Mikroorganismen und Zellkulturen GmbH (DSMZ), Inhoffenstrasse 7B, 38124 Braunschweig, Germany, where they are recorded under the following registration numbers: Strain NCFM (DSM33840), deposited on 15 Mar. 2021 and Strain BI-04 (DSM33525), deposited on 19 May 2020. These bacterial strains are commercially available from DuPont Nutrition Biosciences ApS.

Inclusion of the individual strains in a DFM mixture can be in proportions varying from 1% to 99% and, preferably, from 25% to 75%.

Suitable dosages of the one or more DFM in water line compositions may range from about 1×10³ CFU/mL water to about 1×10¹⁰ CFU/mL water, 1×10¹¹ CFU/mL water, 1×10¹² CFU/mL water, suitably between about 1×10⁴ CFU/mL water to about 1×10⁸ CFU mL water, 1×10⁹ CFU/mL water, 1×10¹⁰ CFU/mL water suitably between about 7.5×10⁴ CFU/mL water to about 1×10⁷ CFU/mL water, or 1×10⁸ CFU/mL water. In another aspect, the DFM may be dosed in water line compositions at more than about 1×10³ CFU/mL water, suitably more than about 1×10⁴ CFU/mL water, suitably more than about 5×10⁴ CFU/mL water, or suitably more than about 1×10⁵ CFU/mL water.

The one or more DFM may be dosed in a water line composition from about 1×10³ CFU/g composition to about 1×10¹³ CFU/g composition, preferably 1×10⁵ CFU/g composition to about 1×10¹³ CFU/g composition, more preferably between about 1×10⁶ CFU/g composition to about 1×10¹² CFU/g composition, and most preferably between about 3.75×10⁷ CFU/g composition to about 1×10¹¹ CFU/g composition. In another aspect, the DFM may be dosed in a water line composition at more than about 1×10⁵ CFU/g composition, preferably more than about 1×10⁶ CFU/g composition, and most preferably more than about 3.75×10⁷ CFU/g composition. In one embodiment, the DFM is dosed in the water line composition at more than about 2×10⁵ CFU/g composition, suitably more than about 2×10⁶ CFU/g composition, suitably more than about 3.75×10⁷ CFU/g composition.

In another embodiment, the dosage range for inclusion of one or more DFMs administered in drinking water, such as via a water line is about 1×10³ CFU/animal/day to about 1×10¹⁵ CFU/animal/day, for example, about 1×10³ CFU/animal/day, 1×10⁴ CFU/animal/day, 1×10⁵ CFU/animal/day, 1×10⁶ CFU/animal/day, 1×10⁷ CFU/animal/day, 1×10⁸ CFU/animal/day, 1×10⁹ CFU/animal/day 1×10¹⁰ CFU/animal/day, 1×10¹¹ CFU/animal/day, 1×10¹² CFU/animal/day, 1×10¹³ CFU/animal/day, 1×10¹⁴ CFU/animal/day, or 1×10¹⁵ CFU/animal/day, inclusive of all dosages falling in between these values.

### C. Thickening Agents

In other aspects, the water line compositions disclosed herein can contain a buffer sufficient to maintain pH of water at about or greater than about 6.5, one or more DFMs, and optionally one or more thickening agents. In accordance with some embodiments of the invention, enhanced suspension in solution of the buffered DFM-containing water line compositions disclosed herein can be achieved through the use of thickening agents. The DFM can be a bacterium, such as, without limitation, one or more bacteria selected from a Bacillus spp., a Bifidobacterium spp., a Lactobacillus spp., and a Megasphaera spp. (e.g., one or more of Bacillus subtilis, Bacillus amyloliquefaciens, Bacillus velezensis, Bifidobacterium animalis subsp. lactis, Lactobacillus reuteri, Lactobacillus acidophilus, or Megasphaera elsdenii). It is contemplated that the thickening agents may be capable of aiding in maintaining the stability (such as suspension in solution) of the compositions due to their properties. If desired, two or more thickening agents may be employed in the present compositions.

A wide variety of thickening agents are known to those skilled in the art and may be used in the practice of the present invention. In some embodiments, the thickening agent may be an organic thickening agent or an inorganic thickening agent. In some embodiments, the organic thickening agents are polymeric thickening agents. The term “polymer”, as used herein, refers to molecules formed from the chemical union of two or more units. Accordingly, included within the term “polymer” are, for example, dimers, trimers and oligomers. The polymer may be synthetic, naturally-occurring or semisynthetic. In one non-limiting form, the term “polymer” refers to molecules which comprise 10 or more repeating units. Suitable polymeric thickening agents for use in the present compositions include, for example, starches, gums, pectin, casein, gelatin, phycocolloids and synthetic polymers. Exemplary of the foregoing materials are, for example, alginates and salts and derivatives thereof, including, for example, sodium alginate and propylene glycol alginate, acacia, carrageenan, guar gum, karaya gum, locust bean gum, tragacanth, xanthan gum, celluloses and salts and derivatives thereof including, for example, carboxymethylcellulose, carboxymethylcellulose sodium, carboxymethylcellulose calcium, ethylcellulose, hydroxyethylcellulose, methylcellulose, hydroxypropyl cellulose, hydroxypropyl methylcellulose, microcrystalline cellulose and powdered cellulose, hyaluronic acid and salts thereof such as, for example, sodium hyaluronate, gelatin and polydextrose.

The amount of thickening agent employed in the present compositions may vary and depends, for example, on the particular polymer and solvent employed, the quantity DFMs and buffer, the desired viscosity of the final composition and the like. Generally speaking, the thickening agent may be employed in an amount to provide the compositions with a desired viscosity and/or suspension in solution. In one embodiment, the thickening agent may be employed in an amount which ranges from about 0.01% to about 50%, and all combinations and subcombinations of ranges and specific amounts therein. In other embodiments, the thickening agent may be employed in an amount of from about 0.05% to about 3%, such as about 0.15% to about 0.6%, such as about 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, 1.9%, 2%, 2.5%, or 3%, inclusive of all values falling in between these percentages. In one embodiment, the thickening agent comprises xanthan gum.

### D. Means for Inactivating Chloramine or Chlorine

In some aspects, the water line compositions disclosed herein can contain a buffer sufficient to maintain pH of water at about or greater than about 6.5, one or more DFMs, optionally one or more thickening agents (such as any thickening agent disclosed herein), and further optionally one or more means for inactivating chloramine or chlorine, for example, a dechlorination agent. The DFM can be a bacterium, such as, without limitation, one or more bacteria selected from a Bacillus spp., a Bifidobacterium spp., a Lactobacillus spp., and a Megasphaera spp. (e.g., one or more of Bacillus subtilis, Bacillus amyloliquefaciens, Bacillus velezensis, Bifidobacterium animalis subsp. lactis, Lactobacillus reuteri, Lactobacillus acidophilus, or Megasphaera elsdenii).

Chlorine kills pathogens such as bacteria and viruses by breaking the chemical bonds in their molecules. Disinfectants that are used for this purpose consist of chlorine compounds which can exchange atoms with other compounds, such as enzymes in bacteria and other cells. When enzymes come in contact with chlorine, one or more of the hydrogen atoms in the molecule are replaced by chlorine. This causes the entire molecule to change shape or fall apart. When enzymes do not function properly, a cell or bacterium will die.

During livestock production, many different water sources can be used for animal consumption, such as water wells, fountains, shallow wells, semi-artesian and artesian wells, municipal water supplies, lakes and creeks. The use of these water sources exposes the animals to pathogenic virus and bacteria through contamination by wild animals and human waste. It is therefore necessary to sanitize such water sources by adding oxidizing sanitizers such as chlorine, peroxide, bromine, and the like to water. Chlorine and chloramine are the major disinfectants used in public water systems to kill bacteria and other microbes in drinking water supplies. Less commonly, utilities as well as farmers use other disinfectants, such as chlorine dioxide (ClO₂). However, all these treatments can result in water that can decrease the viability or kill DFMs included in the water line compositions disclosed herein.

Accordingly, the water line compositions disclosed herein can additionally include one or more means for inactivating chloramine or chlorine present in the water. Means for inactivating chloramine or chlorine can include, without limitation, adsorption dechlorination (e.g., with activated carbon, such as granular activated carbon (GAC)), ultraviolet dechlorination (i.e. using broad spectrum ultraviolet irradiation to dissociate free chlorine and chloramines), or chemical dechlorination. With regard to chemical dechlorination, sulfur dioxide is most commonly used but alternatives include, without limitation, sodium metabisulfite, sodium bisulfite, hydrogen peroxide, and ascorbic acid. Sodium metabisulfite and sodium bisulfite are mainly used in small facilities because these materials are more difficult to control compared to sulfur dioxide. Hydrogen peroxide is not frequently used because it is dangerous to handle (see Environmental Protection Agency. 2000. Wastewater Fact Sheet. Dechlorination. EPA 832-F-00-022. Washington, DC: U.S. Environmental Protection Agency).

Another means for inactivating chlorine, chloramine or chlorine dioxide present in water is treatment with ascorbic acid or sodium ascorbate (i.e. two forms of Vitamin C) or their derivatives (e.g. iso-ascorbic acid). Approximately 2.5 parts of ascorbic acid are required for neutralizing 1-part chlorine. Sodium ascorbate will also neutralize chlorine. It is pH neutral and will not change the pH of the treated water. Approximately 2.8 parts of sodium ascorbate are required to neutralize 1-part chlorine (see Land et al. 2005. “Using Vitamin C to Neutralize Chlorine in Water Systems,” 0523 1301—SDTDC, Washington DC: U.S. Department of Agriculture (USDA) Forest Service). Ascorbic acid and derivatives will also neutralize chloramine and chlorine dioxide.

Another means for inactivating chlorine, chloramine or chlorine dioxide present in water is treatment with metal ions. Aqueous chlorine rapidly and stoichiometrically oxidizes iron (II), while manganese (U) reaction rate increases with increasing pH and is effective for MnO(OH)₂ precipitation above pH=7.5.

Fukayama et al. (Environmental Health Perspectives, 1986, 69:267-274) investigated the reaction of aqueous chlorine and chlorine dioxide with food compounds and reported carbohydrates, lipids, amino acids, peptides and proteins to react with chlorine. Tan et al. (Mutat Res. 1987 August; 188(4):259-66) found that of 20 amino acids and three peptides (L-aspartyl-L-phenylalanine methyl ester (aspartame), L-glycyl-L-tryptophan and L-tryptophylglycine), only few were reactive with C102 at pH 6.0. Among amino acids and peptides, the reactivity of cysteine, tryptophan, tyrosine, L-glycyl-L-tryptphan and L-tryptophylglycine were rapid while histidine, proline, and hydroxyproline had measurable rates. Other amino acids and aspartame did not show reactivity with C002. Hence, cysteine, tryptophan, tyrosine, methionine, histidine, proline, and hydroxyproline represent other means to neutralize chlorine compounds in water. Other antioxidants, e.g. glutathione and polyphenols (e.g. from fruits or leaves) can also efficiently be used to inactive chlorine compounds.

### E. Wetting and Dispersing Agents

In some aspects, the water line compositions disclosed herein can contain a buffer sufficient to maintain pH of water at about or greater than about 6.5, one or more DFMs, optionally one or more thickening agents (such as any thickening agent disclosed herein), optionally one or more means for inactivating chloramine or chlorine, for example, a dechlorination agent, and further optionally one or more wetting and/or dispersing agents. The DFM can be a bacterium, such as, without limitation, one or more bacteria selected from a Bacillus spp., a Bifidobacterium spp., a Lactobacillus spp., and a Megasphaera spp. (e.g., one or more of Bacillus subtilis, Bacillus amyloliquefaciens, Bacillus velezensis, Bifidobacterium animalis subsp. lactis. Lactobacillus reuteri, Lactobacillus acidophilus, or Megasphaera elsdenii).

As used herein, the term “wetting agent” means a compound used to aid in attaining intimate contact between solid particles and liquids. Useful wetting agents include by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters or polysorbates (e.g., TWEEN®), polyethylene glycols, polyoxyethylene stearates, phosphates, sodium lauryl sulphate, poloxamer, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (or PVP). Tyloxapol (a nonionic liquid polymer of the alkyl aryl polyether alcohol type, also known as superinone or triton) is another useful wetting agent, combinations thereof and other such materials known to those of ordinary skill in the art.

The amount of wetting agent employed in the present compositions may vary and depends, for example, on the particular polymer and solvent employed, the quantity DFMs and buffer, the desired speed of dissolution of solids into liquids of the final composition and the like. Generally speaking, the wetting agent may be employed in an amount to provide the speedier dissolution of solids into solution. In one embodiment, the wetting agent may be employed in an amount which ranges from about 0.01% to about 50%, and all combinations and subcombinations of ranges and specific amounts therein. In other embodiments, the wetting agent may be employed in an amount of from about 0.05% to about 3%, such as about 0.15% to about 0.6%, such as about 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, 1.9%, 2%, 2.5%, or 3%, inclusive of all values falling in between these percentages.

Wetting agents may also be used for alleviating soil water repellency as described by Song et al. (Nanomaterials 11(10): 2577 (2021)).

In the present invention, the water line compositions can further comprise a dispersant or a dispersing agent. As used herein, a “dispersant” or “dispersing agent” is an agent capable of stabilizing a suspension and limiting aggregation of the suspended particulates. Suitable dispersing agents are non-toxic pharmaceutically acceptable dispersing agents and include but are not limited to thickening agents (such as any of the thickening agents described herein).

Exemplary dispersing agents include, by way of example and not limitation, silicon dioxides, and derivatives of silicon dioxides, such as alkylated silica gels and colloidal silicon dioxide, such as those available under the trade name Aerosil (e.g., Aerosil 130, 200, 300, 380, O, OX50, TT600, MOX 80, MOX 170, LK 84 and methylated Aerosil R 972) or CAB-O-SIL®. Other dispersing agents include, but are not limited to, silicon dioxides and derivatives of silicone dioxides and compatible mixtures thereof, more preferably colloidal silicon dioxide.

In another aspect, the dispersing agents may be bentonite, a hydrated aluminum silicate found in certain types of clay and which is in the form of colloidal particles of about 50-150 microns and numerous particles of about 1-2 microns. A similar dispersing agent is kaolin, another type of aluminum silicate, also found in certain naturally occurring clays. Other dispersing agents may include hectorite, magnesium aluminium silicate, magnesium oxide. Preferred dispersing agents include but are not limited to bentonite, kaolin, magnesium aluminium silicate, magnesium oxide, and compatible mixtures thereof.

In another aspect, the dispersing agents are also thickening agents (such as any of the thickening agents described above). Suitable thickening agents include but are not limited to dextrin, alginates, propylene glycol alginate, and zinc stearate. Also finding use as thickening agents are water-soluble celluloses and cellulose derivatives including, among others, alkyl celluloses, such as methyl-, ethyl-, and propyl-celluloses; hydroxyalkyl-celluloses, such as hydroxypropyl celluloses and hydroxypropylalkylcelluloses; acylated celluloses, such as cellulose acetates, cellulose acetatephthallates, cellulose-acetate succinates and hydroxypropylmethyl-cellulose phthalates; and salts thereof, such as sodium carboxymethyl celluloses. Useful celluloses are available under the tradenames Klucel and Methocel. Preferred thickening agents include but are not limited to alginates, hydroxypropyl celluloses, hydroxypropylmethylcellulose phthalates, sodium carboxymethyl celluloses, and compatible mixtures thereof.

Other dispersing agents suitable for use in the water line formulations will be known to those of ordinary skill in the art, and are to be included within the scope of the compositions described herein (see, e.g., Handbook of Pharmaceutical Excipients, 4th Ed, (Kibbe, A. H. ed.) Washington D.C., American Pharmaceutical Association (2003); incorporated by reference herein).

The amount of dispersing agent employed in the present compositions may vary and depends, for example, on the particular polymer and solvent employed, the quantity DFMs and buffer, the desired uniformity of solids and liquids of the final composition and the like. Generally speaking, the dispersing agent may be employed in an amount to provide a stable suspension with limited aggregation of solids. In one embodiment, the dispersing agent may be employed in an amount which ranges from about 0.01% to about 50%, and all combinations and subcombinations of ranges and specific amounts therein. In other embodiments, the dispersing agent may be employed in an amount of from about 0.05% to about 3%, such as about 0.15% to about 0.6%, such as about 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, 1.9%, 2%, 2.5%, or 3%, inclusive of all values falling in between these percentages.

### III. Methods

### A. Methods for Preparing a Composition for Maximizing Delivery of Live Direct Fed Microbial (DFM) Cells Through a Water Line

Further provided herein are methods for preparing a composition for maximizing delivery of live direct fed microbial (DFM) cells through a water line. The method comprises mixing a buffer sufficient to maintain pH of water at about or greater than about 6.5 (such as any of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, or greater; and one or more direct fed microbials (DFMs; such as any of the DFMs, genera, species, or strains disclosed herein) with water. The water can come from a municipal water source, well water, surface water, and/or collected rain water. The DFM can be a bacterium, such as, without limitation, one or more bacteria selected from a Bacillus spp., a Bifidobacterium spp., a lactobacillus spp., and a Megasphaera spp. (e.g., one or more of Bacillus subtilis, Bacillus amyloliquefaciens, Bacillus velezensis, Bifidobacterium animalis subsp. lactis, Lactobacillus reuteri, Lactobacillus acidophilus, or Megasphaera elsdenii).

In some embodiments, the DMFs can be freeze dried or lyophilized. As used herein, “freeze-dried” refers to a one or more DFM compositions disclosed herein having the characteristics described herein and further having substantially no water present, and in one embodiment, no detectable water. Methods for freeze-drying a composition are known and routinely used. The phrase “freeze-drying” is used synonymously with “lyophilization.” A method for freeze-drying a composition may include one or more pretreatments (e.g., concentrating, addition of a cryoprotectant, increasing the surface area of a composition), freezing the composition, and drying (e.g., exposing the composition to a reduced atmospheric pressure to result in sublimation of the water present in the composition).

In some embodiments, freeze dried or lyophilized DFMs can include a cryoprotectant. A “cryoprotectant” is a compound that maintains the viability of microbes when frozen. Cryoprotectants are known in the art and used routinely to protect microbes when exposed to freezing conditions. Examples include, but are not limited to, amino acids such as alanine, glycine, proline, simple sugars such as sucrose, glucose, lactose, ribose, and trehalose, and other compounds such as dimethyl sulfoxide (DMSO), and glycerol. The amount of cryoprotectant present in a composition described herein may vary depending on the cryoprotectant used and the temperature to be used for freezing (e.g., −20° C., −80° C., or a different temperature) The amount of cryoprotectant that can be used is known to the skilled person or may be easily determined using routine experimentation. In one embodiment, a composition of the present invention may include glycerol at a concentration of 10%.

Other known cryoprotectants include, for instance, D-Mannitol, D-Sorbitol, D-Glucose, casein hydrolysate, sucrose, gelatin, non-fat skim milk, starch hydolysate, fetal calf serum, bovine serum albumin, or combinations of 1, 2, 3, or 4 of the above cryoprotectants. Other cryoprotectants are also known. A cryoprotectant useful herein maintains the viability of microbes when subjected to freeze-drying conditions, milling or grinding, and/or when stored as a freeze-dried composition. Milling, also referred to as grinding, is a process that physically changes a material into smaller particles. Methods for milling freeze-dried compositions are known to the skilled person, and can occur at various temperatures, e.g., at or below 0° C., or above 0° C. A cryoprotectant useful herein results in a freeze-dried composition that is friable. As used herein, a “friable” composition refers to a composition that can be easily milled to result in a fine powder. In one embodiment, a freeze-dried composition described herein that is friable is one that results in a powder that can be subsequently used to produce a tablet. In one embodiment, a useful powder may have size, density, flow, and compression characteristics suitable for production of tablets or encapsulation.

The total cryoprotectant used to produce a freeze-dried composition may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (vol/vol) of the final concentration of a mixture of microbes and the cryoprotectant before freeze-drying the composition. For instance, to produce a composition having a cryoprotectant at a final concentration of 10%, equal volumes of a 20% solution of the cryoprotectant and a mixture of microbes derived from material can be combined and mixed, and then freeze-dried.

The method can further include a step of mixing one or more thickening agents with the buffer sufficient to maintain pH of water at about or greater than about 6.5 and one or more DFMs, including any of the thickening agents described herein, for example, xanthan gum. Additionally, the method can further include a step of mixing one or more means for inactivating chloramine or chlorine present in the water (such as any such mean disclosed herein) with the buffer sufficient to maintain pH of water at about or greater than about 6.5 and one or more DFMs and optionally one or more thickening agents.

In one embodiment, the method provides a “stock” solution that is fed into a water line for delivery to livestock. This stock solution is more highly concentrated with regard to the amount of buffer, DFMs, and optionally xanthan gum than the solution that is eventually pumped into a water line. In some embodiments, the stock solution has between about 1-2000 mM

### B. Methods for Delivering One or More DFMs to a Subject Via a Water Line

In still other aspects, provided herein are methods for delivering one or more direct fed microbials (DFMs) to a subject comprising administering any of the buffered DFM-containing water line compositions disclosed herein through a water line over a distance, wherein the DFMs are delivered to the subject when the subject drinks from the water line or when the DFM passively is released by the water line (such as, for example, as with irrigation systems used for supplying moisture to plants). The DFM can be a bacterium, such as, without limitation, one or more bacteria selected from a Bacillus spp., a Bifidobacterium spp., a Lactobacillus spp., and a Megasphaera spp. (e.g., one or more of Bacillus subtilis, Bacillus amyloliquefaciens, Bacillus velezensis, Bifidobacterium animalis subsp. lactis, Lactobacillus reuteri, Lactobacillus acidophilus, or Megasphaera elsdenii). In some embodiments, the subject is a livestock such as, without limitation, poultry, swine, or a ruminant (such as a cow). In other embodiments, the subject is a plant, for example, a crop plant such as, without limitation soy, cotton, canola, maize, wheat, sunflower, sorghum, alfalfa, barley, millet, rice, tobacco, fruit and vegetable crops, cannabis, and turf grass.

In some embodiments, the one or more DFMs exhibit decreased settling in the water line compared to identical DFMs that are not administered in one of the buffered DFM-containing water line compositions disclosed herein. In some embodiments, the one or more DFMs exhibit any of about 5%, 10%, 15%, 20%, 25%, 30%, 35%, 40%, 45%, 50%, 55%, 60%, 65%, 70%, 75%, 80%, 85%, 90%, 95%, or 100% less settling, inclusive of values falling in between these percentages, compared to identical DFMs that are not administered in one of the buffered DFM-containing water line compositions disclosed herein.

In other embodiments, the one or more DFMs exhibit increased survival and/or viability in the water line compared to identical DFMs that are not administered in one of the buffered DFM-containing water line compositions disclosed herein. In some embodiments, the one or more DFMs exhibit any of about 5%, 10%, 15%, 20%, 25%, 30%, 35%, 40%, 45%, 50/%, 55%, 60%, 65%, 70%, 75%, 80%, 85%, 90%, 95%, 100%, 105%, 110%, 115%, 120%, 125%, 130%, 135%, 140%, 145%, 150% or more increased survival and/or viability, inclusive of values falling in between these percentages, compared to identical DFMs that are not administered in one of the buffered DFM-containing water line compositions disclosed herein.

In yet another embodiment, the one or more DFMs exhibit increased “benefit potential” in the water line compared to identical DFMs that are not administered in one of the buffered beneficial microbe-containing water line compositions disclosed herein. “Benefit potential” is a composite feature of the viability and the settling properties of the DFMs, e.g. as determined as the concentration of floating viable cells. In some embodiments, the one or more beneficial microbes exhibit any of about 5%, 10%, 15%, 20%, 25%, 300/a, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150% or more increased “benefit potential”, inclusive of values falling in between these percentages, compared to identical beneficial microbes that are not administered in one of the buffered DFM-containing water line compositions disclosed herein.

The water line can be from about 20 meters to about 200 meters in length, such as any of about 30-190 meters, 40-180 meters, 50-150 meters, 60-140 meters, 70-130 meters, 80-120 meters, 90-110 meters, or any of about 30 meters, 35 meters, 40 meters, 45 meters, 50 meters, 55 meters, 60 meters, 65 meters, 70 meters, 75 meters, 80 meters, 85 meters, 90 meters, 95 meters, 100 meters, 105 meters, 110 meters, 115 meters 120 meters, 125 meters, 130 meters, 135 meters, 140 meters, 145 meters, 150 meters, 155 meters, 160 meters, 165 meters, 170 meters, 175 meters, 180 meters, 185 meters, 190 meters, 195 meters, 200 meters, or more in length, inclusive of values falling in between these distances.

### IV. Kits

In other aspects, provided herein are kits comprising one or more components of the buffered DFM-containing water line compositions disclosed herein as well as written instructions for combining the kit components with water for water line delivery of the one or more DFMs. The DFM can be a bacterium, such as, without limitation, one or more bacteria selected from a Bacillus spp., a Bifidobacterium spp., a Lactobacillus spp., and a Megasphaera spp. (e.g., one or more of Bacillus subtilis, Bacillus amyloliquefaciens, Bacillus velezensis, Bifidobacterium animalis subsp. lactis, Lactobacillus reuteri, Lactobacillus acidophilus, or Megasphaera elsdenii). Any suitable container can be used to package the kit components including, without limitation, a sachet, a bag, a or a box.

The kit components or water line compositions disclosed herein can be delivered as bulk material or pre-portioned into discrete quantities. The advantage of providing the material as bulk, e.g. in glass bottles, metal cans, moisture-impermeable bags or any other container, is that a user can measure out the desired product amount specific for the desired use. Measuring out the quantity can either be accomplished by mass or by volume. However, in case of bulk delivery, a tight re-sealing of the bulk container can be provided due to the hygroscopic nature of the product.

Alternatively, the product can be delivered in discrete units, e.g. for one-time use for a specified number of livestock. If the livestock number would be larger than specified, proper multiples of the discrete packaging can be used closest to the required total amount. One feature of the discrete unit is that de-mixing of the different components of the blend are not of concern, as each unit is used up in one application and contains the optimally balanced components of the formulation. Hence the different components of the formulation can either be blended or combined in a homogenous composite. Discrete portions of the formulation can be achieved by delivering it in the form of water-dissolvable tablets, water-dissolvable pods (e.g. powder distributed in cold water soluble PVA film bags) or in water-dissolvable (soft gel) capsules (e.g. in pullulan capsules), where the complete package would be dissolved and rehydrated in water, or in form of glass/metal/plastic vials or ampoules, as well as pouches, sachets, sticks, bags and other containers, where only the content would be rehydrated in water.

The invention can be further understood by reference to the following examples, which are provided by way of illustration and are not meant to be limiting.

### EXAMPLES

### Example 1: Settling of Gram-Positive Aerotolerant Anaerobes at Different Cell Densities

Freeze-dried powder of Lactobacillus reuteri LAB1 was dissolved in ultrapure water to give a master solution of approximately 10 OD. Subsequently, 15 mL Eppendorf tubes were filled with 1 mL of 1 M equimolar K₂HPO₄/KH₂PO₄ phosphate buffer, ultrapure water and the corresponding volume of the master solution ad 10 mL, resulting in OD values at λ=600 nm of 0.9976, 0.8456, 0.6670, 0.4834, 0.2872, 0.1858, 0.1620, 0.1476 and 0.1295, after subtracting a blank of ultrapure water. Resulting pH in samples was around 6.6, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

For measurement of OD₆₀₀, a Varian Cary 300 Bio UV/Visible spectrophotometer equipped with a temperature controller (Varian Medical Systems, Palo Alto, CA) was set to a wavelength of λ=600 nm. The instrument was equipped with 12 cuvette slots, and each slot was read sequentially in an approximately 10 minutes cycle and the measured OD₆₀₀ values were recorded. Fisherbrand Cuvettes (#14955125, Thermo Fisher Scientific, Waltham, MA) with a capacity of 4.5 mL were filled with 3.952 mL from the stock solution. The cuvette temperature was controlled at 23° C. during the experiment. At the start of the experiment, the cuvettes were thoroughly mixed and subsequently closed with a lid (Fisherbrand cuvette cap square LDPE 19 mm, #14385999, Thermo Fisher Scientific, Waltham, MA).

At the end of the experiment, all measured OD values X above 0.9000 were corrected according to

$${X}_{corr} = 1.1133 * X \hat{} 4 - 4.82 * X \hat{} 3 + 8.1997 * X \hat{} 2 - 5.1792 * X + 1.7064$$

to account for measurements outside the linear range of the spectrophotometer. Subsequently, from all samples the blank measurements of a 100 mM phosphate buffer in ultrapure water against only ultrapure water at the respective time point and temperature was subtracted. Of each corrected measurement set, the maximum value Xmax(i) was determined, and used to calculate a normalization factor F for each of the various concentrated cultures i according to

$$F ( i ) = 1 / X max ( i ) .$$

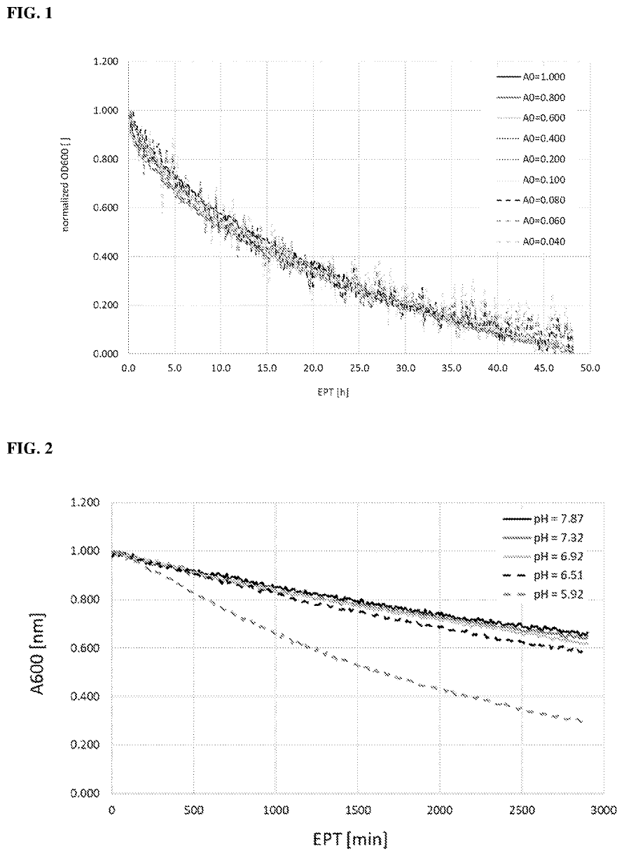

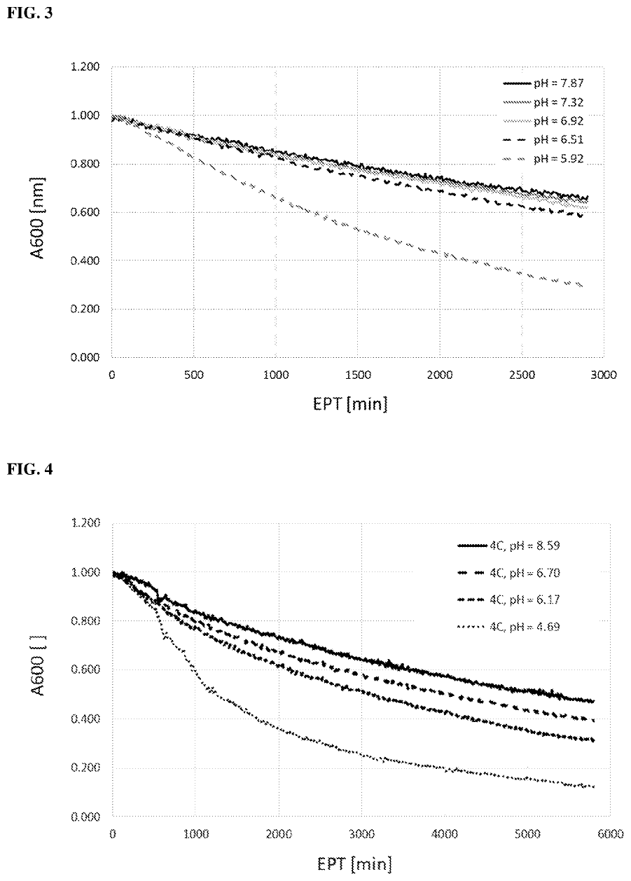

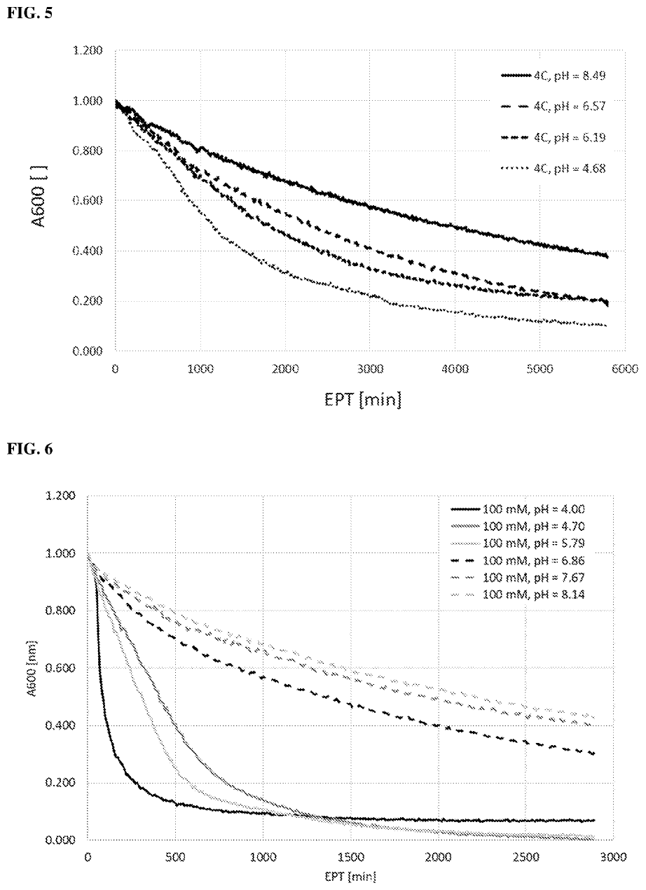

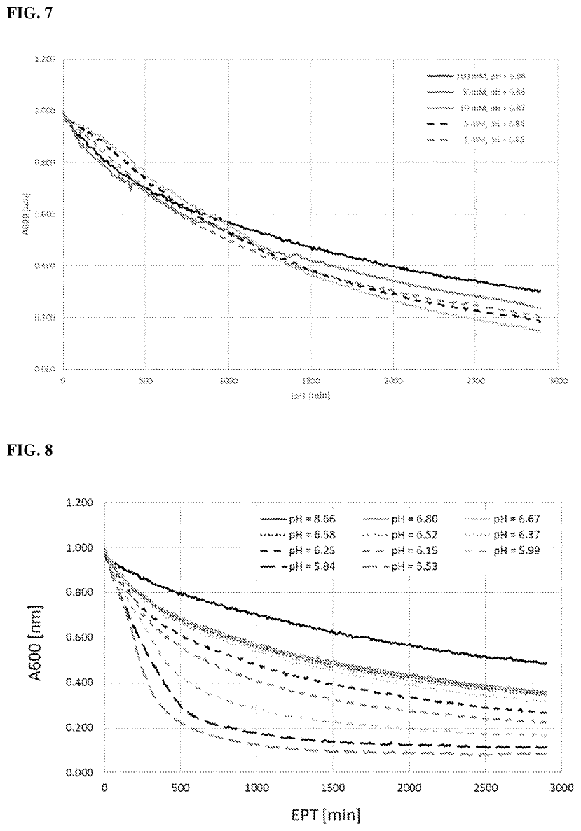

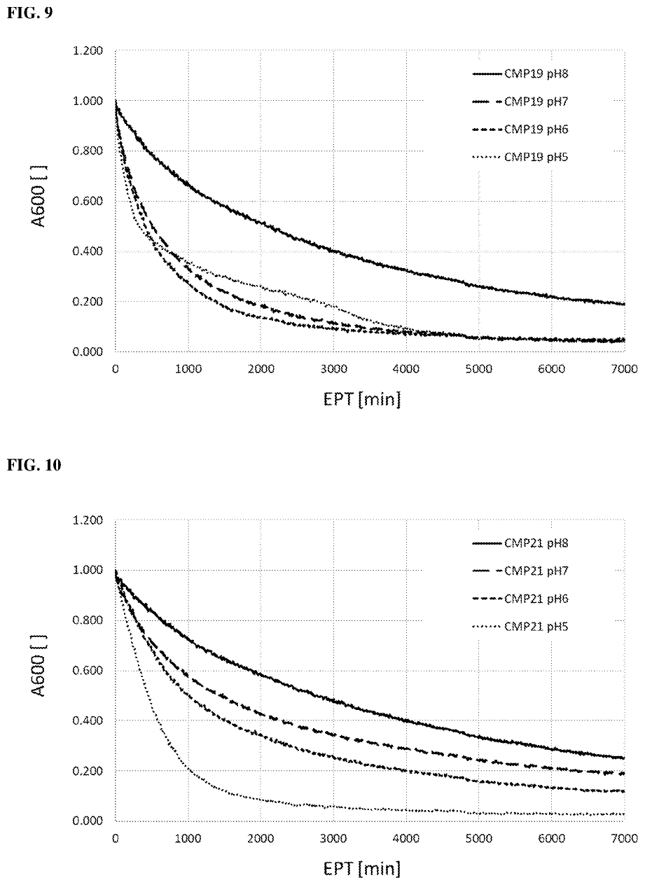

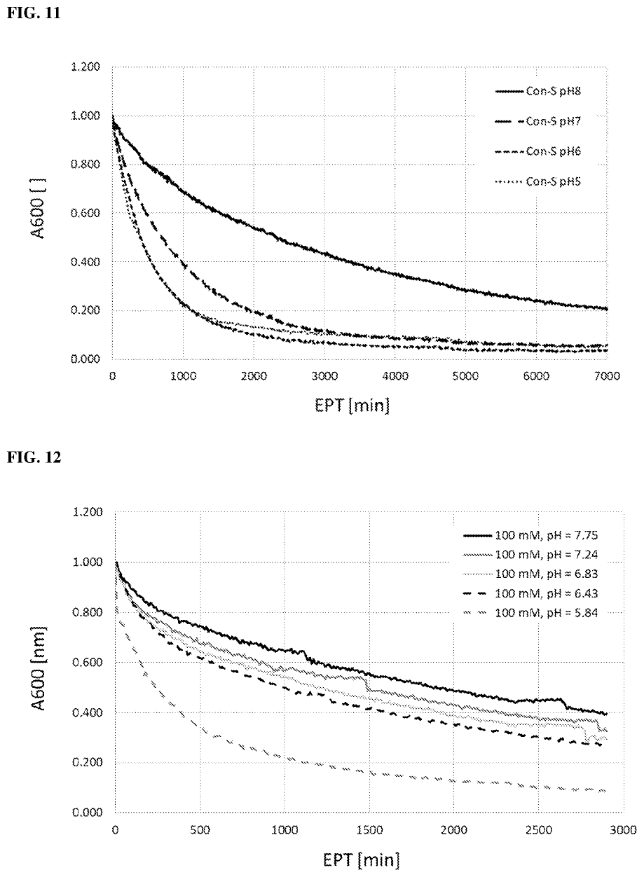

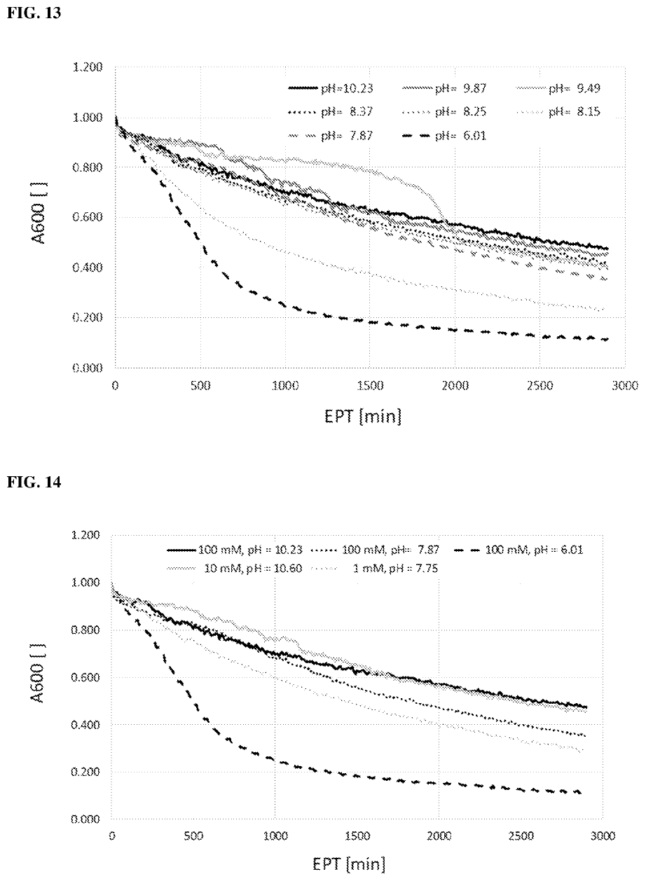

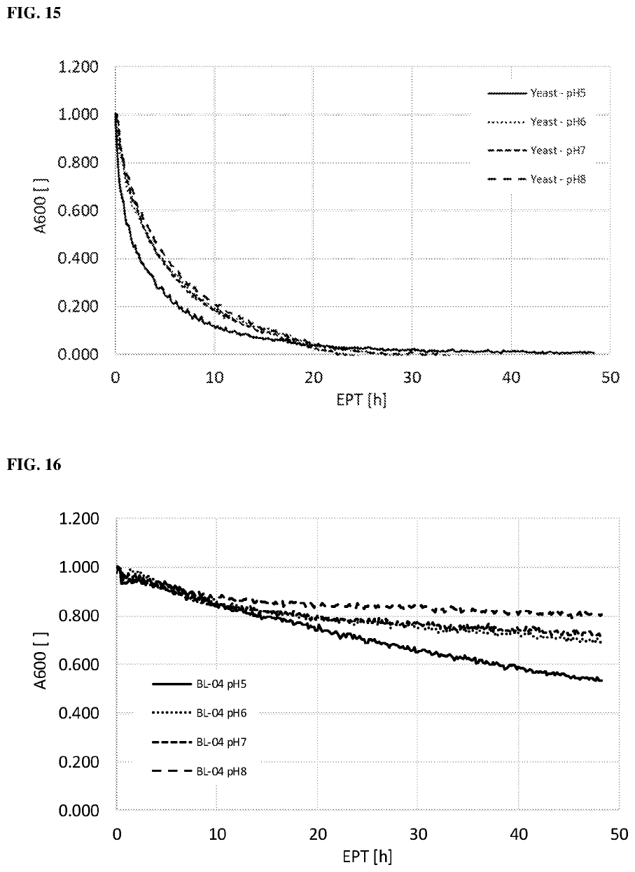

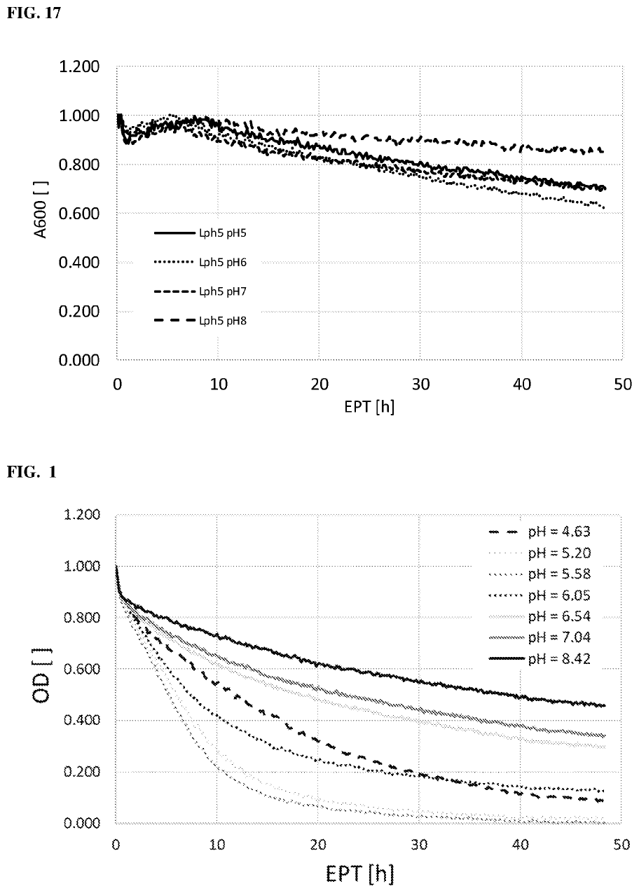

The resulting OD₆₀₀ time curves for the different cell concentrations are depicted in FIG. 1. The 9 normalized OD₆₀₀ time curves are very similar, indicating that settling kinetics of cells in the investigated OD range are concentration independent and no or only minimal aggregation of cells occurs.

### Example 2: Settling of Gram-Positive Aerotolerant Anaerobes at 4° C. at Different pH Values

This example demonstrates that Lactobacillus reuteri LAB1 at 4° C. settles faster at lower than at higher pH.

Freeze-dried powder of Lactobacillus reuteri LAB1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.863, 0.857, 0.853, 0.856 and 0.843 after subtracting a blank with ultrapure water. Resulting pH in samples was 5.92, 6.51, 6.92, 7.32 and 7.87, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 4° C. during the experiment. Correction and normalization of OD values were performed as described in Example 1.

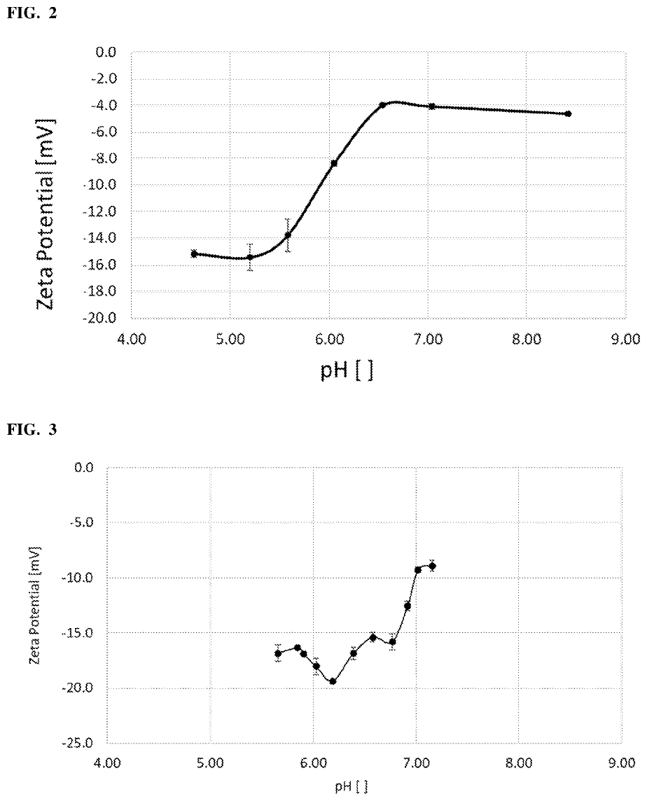

The resulting OD₆₀₀ settling time curves at 4° C. in different pH suspensions are depicted in FIG. 2. The normalized OD₆₀₀ time curves of the cell suspensions at various pH indicate that Lactobacillus reuteri LAB1 at 4° C. settles faster at lower than at higher pH.

### Example 3: Settling of Gram-Positive Aerotolerant Anaerobes at 4° C. at Different pH Values

This example demonstrates that Lactobacillus reuteri CMP19 at 4° C. settles faster at lower than at higher pH.

Freeze-dried powder of Lactobacillus reuteri CMP19 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.728, 0.705, 0.698 and 0.705 after subtracting a blank with ultrapure water. Resulting pH in samples was around 4.81, 6.28, 6.70 and 8.48, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 4° C. during the experiment. Correction and normalization of OD values were performed as described in Example 1.

The resulting OD₆₀₀ settling time curves at 4° C. in different pH suspensions are depicted in FIG. 3. The normalized OD₆₀₀ time curves of the cell suspensions at various pH indicate that Lactobacillus reuteri CMP19 at 4° C. settles faster at lower than at higher pH.

### Example 4: Settling of Gram-Positive Aerotolerant Anaerobes at 4° C. at Different pH Values

This example demonstrates that Lactobacillus reuteri CMP21 at 4° C. settles faster at lower than at higher pH.

Freeze-dried powder of Lactobacillus reuteri CMP21 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.861, 0.895, 0.877 and 0.875 after subtracting a blank with ultrapure water. Resulting pH in samples was around 4.69, 6.17, 6.70 and 8.59, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 4° C. during the experiment. Correction and normalization of OD values were performed as described in Example 1.

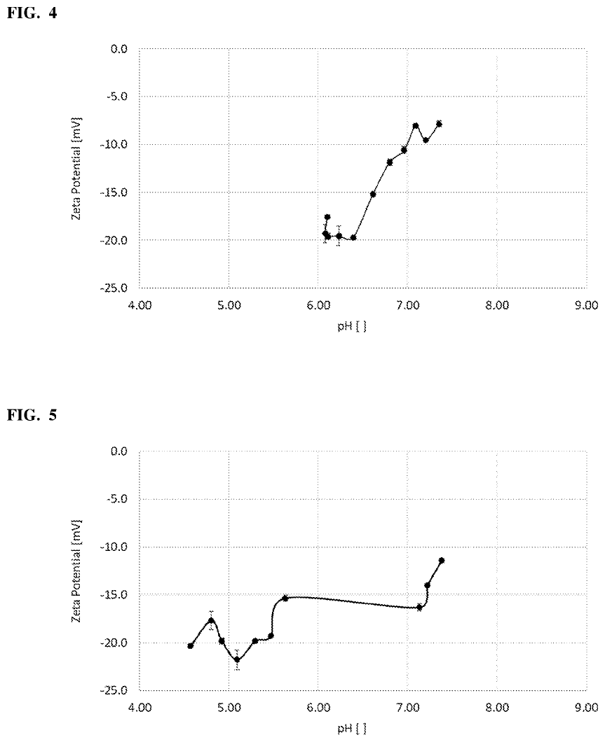

The resulting OD₆₀₀ settling time curves at 4° C. in different pH suspensions are depicted in FIG. 4. The normalized OD₆₀₀ time curves of the cell suspensions at various pH indicate that Lactobacillus reuteri CMP21 at 4° C. settles faster at lower than at higher pH.

### Example 5: Settling of a Three-Strain Consortium of Gram-Positive Aerotolerant Anaerobes at 4° C. at Different pH Values

The example demonstrates that the three-strain Lactobacillus reuteri-containing Consortium-S(ConS) settles faster at lower than at higher pH at 4° C.

Freeze-dried powder of ConS was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.824, 0.808, 0.777 and 0.798 after subtracting a blank with ultrapure water. Resulting pH in samples was around 4.68, 6.19, 6.57 and 8.49, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 4° C. during the experiment. Correction and normalization of OD values were performed as described in Example 1.

The resulting OD₆₀₀ settling time curves at 23° C. in different pH suspensions are depicted in FIG. 5. The normalized OD₆₀₀ time curves of the cell suspensions at various pH indicate that ConS at 4° C. settles faster at lower than at higher pH.

### Example 6: Settling of Gram-Positive Aerotolerant Anaerobes at 23° C. at Different pH Values

This example demonstrates that Lactobacillus reuteri LAB-1 at 23° C. settles faster at lower than at higher pH.

Freeze-dried powder of Lactobacillus reuteri LAB-1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 1.013, 0.971, 0.964, 0.948, 0.938 and 0.952 after subtracting a blank with ultrapure water. Resulting pH in samples was 4.00, 4.70, 5.79, 6.86, 7.67 and 8.14, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

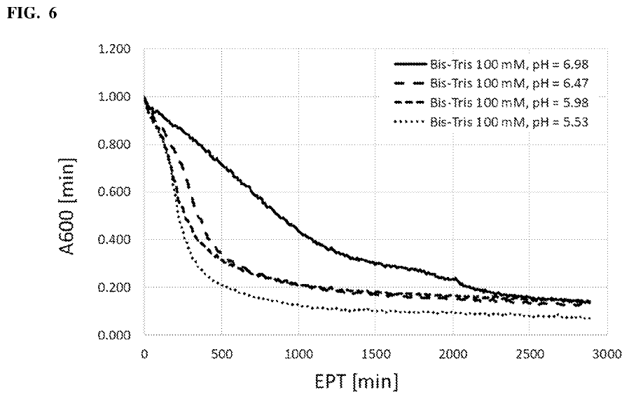

The resulting OD₆₀₀ settling time curves at 23° C. in different pH suspensions are depicted in FIG. 6. The normalized OD₆₀₀ time curves of the cell suspensions at various pH indicate that Lactobacillus reuteri LAB-1 at 23° C. settles faster at lower than at higher pH.

### Example 7: Settling of Gram-Positive Aerotolerant Anaerobes at 23° C. at Different Buffer Molarities

The example demonstrates that settling velocity of Lactobacillus reuteri LAB-1 at 23° C. is not significantly influenced by the molarity of the mono-/dipotassium phosphate solution, as long as the pH value is of comparable value.

Freeze-dried powder of Lactobacillus reuteri LAB-1 was dissolved in ultrapure water to give a master solution of approximately 10 OD. Subsequently, 15 mL Eppendorf tubes were filled with either 1 mL, 0.5 mL, 0.1 mL, 0.05 mL or 0.01 mL of 1 M phosphate buffer with an equimolar K₂HPO₄/KH₂PO₄ ratio, ultrapure water and the corresponding volume of the master solution ad 10 mL, resulting in OD values at λ=600 nm of 0.948, 0.975, 0.995, 0.986 and 0.997 after subtracting a blank with ultrapure water. Resulting pH in samples was 6.86, 6.86, 6.87, 6.84 and 6.65, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

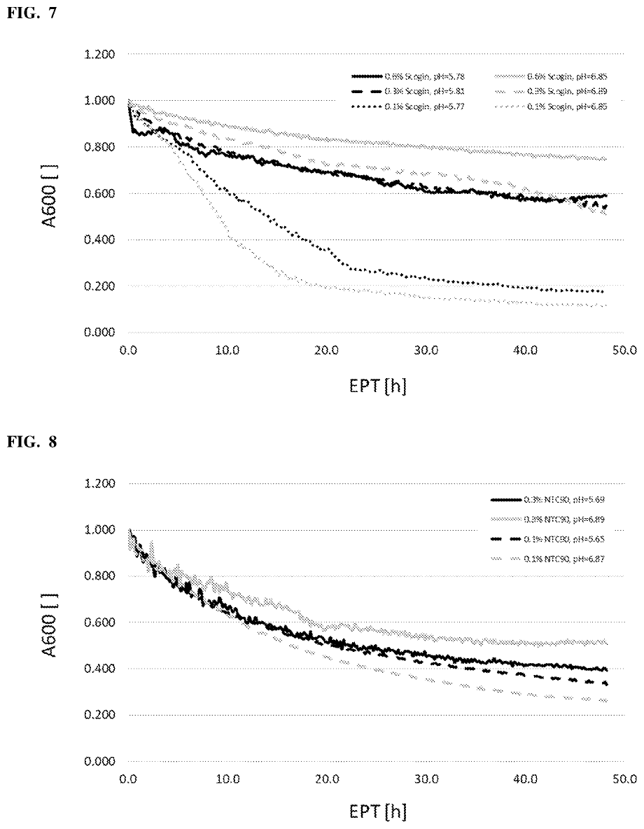

The resulting OD₆₀₀ settling time curves at 23° C. at different phosphate molarities of equimolar di- and monopotassium salts resulting in very similar pH suspensions are depicted in FIG. 7. The normalized OD₆₀₀ time curves of the cell suspensions indicate that settling velocity of Lactobacillus reuteri LAB-1 at 23° C. only exhibit small differences despite significantly different molarities of mono- and dipotassium phosphate in the solutions.

### Example 8: Settling of Gram-Positive Aerotolerant Anaerobes at 23° C. at Different pH Values

This example demonstrates that Lactobacillus reuteri LAB-1 at 23° C. settles faster at lower than at higher pH.

Freeze-dried powder of Lactobacillus reuteri LAB-1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.946, 0.961, 0.944, 0.964, 0.944, 0.960, 0.979, 0.973, 0.958, 0.985 and 0.963 after subtracting a blank with ultrapure water. Resulting pH in samples was 8.66, 6.80, 6.67, 6.58, 6.52, 6.37, 6.25, 6.15, 5.99, 5.84 and 5.53, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

For measurement of OD₆₀₀, a Varian Cary 300 Bio UV/Visible spectrophotometer equipped with a temperature controller (Varian Medical Systems, Palo Alto, CA) was set to a Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH suspensions are depicted in FIG. 8. The normalized OD₆₀₀ time curves of the cell suspensions at various pH indicate that Lactobacillus reuteri LAB-1 at 23° C. settles faster at lower than at higher pH.

### Example 9: Settling of Gram-Positive Aerotolerant Anaerobes at 23° C. at Different pH Values

This example demonstrates that Lactobacillus reuteri CMP19 at 23° C. settles faster at lower than at higher pH.

Freeze-dried powder of Lactobacillus reuteri CMP19 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.709, 0.720, 0.745 and 0.763 after subtracting a blank with ultrapure water. Resulting pH in samples was 4.81, 6.28, 6.70 and 8.48, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH suspensions are depicted in FIG. 9. The normalized OD₆₀₀ time curves of the cell suspensions at various pH indicate that Lactobacillus reuteri CMP19 at 23° C. settles faster at lower than at higher pH.

### Example 10: Settling of Gram-Positive Aerotolerant Anaerobes at 23° C. at Different pH Values

The example demonstrates that Lactobacillus reuteri CMP2/at 23° C. settles faster at lower than at higher pH.

Freeze-dried powder of Lactobacillus reuteri CMP21 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.882, 0.900, 0.918 and 0.908 after subtracting a blank with ultrapure water. Resulting pH in samples was 4.69, 6.17, 6.70 and 8.59, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

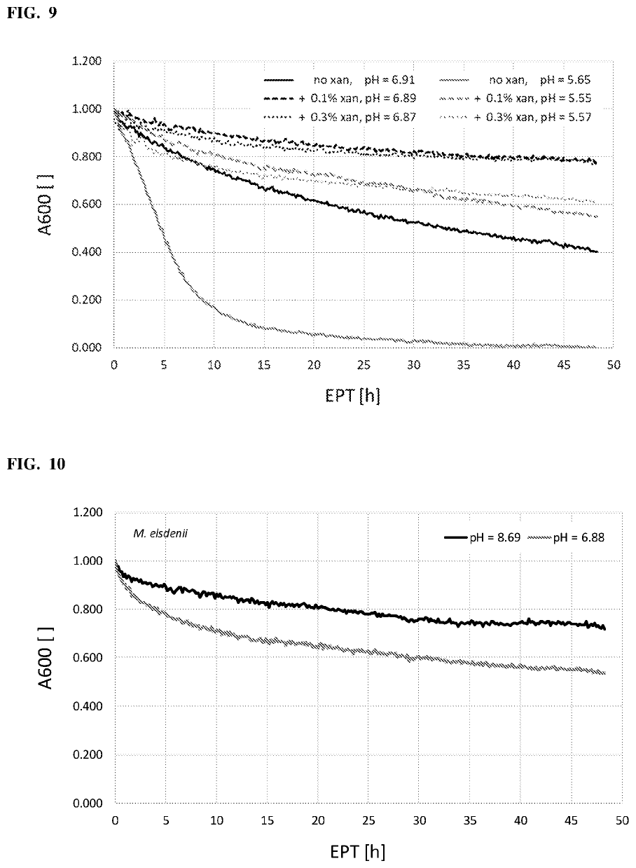

The resulting OD₆₀₀ settling time curves at 23° C. in different pH suspensions are depicted in FIG. 10. The normalized OD₆₀₀ time curves of the cell suspensions at various pH indicate that Lactobacillus reuteri CMP2/at 23° C. settles faster at lower than at higher pH.

### Example 11: Settling of a Three-Strain Consortium of Gram-Positive Aerotolerant Anaerobes at 23° C. at Different pH Values

The example demonstrates that the three-strain Lactobacillus reuteri-containing Consortium-S(ConS) settles faster at lower than at higher pH at 23° C.

Freeze-dried powder of ConS was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.730, 0.742, 0.748 and 0.760 after subtracting a blank with ultrapure water. Resulting pH in samples was 4.68, 6.19, 6.57 and 8.49, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH suspensions are depicted in FIG. 11. The normalized OD₆₀₀ time curves of the cell suspensions at various pH indicate that ConS at 23° C. settles faster at lower than at higher pH.

### Example 12: Settling of Gram-Positive Aerotolerant Anaerobes at 37° C. at Different pH Values

The example demonstrates that Lactobacillus reuteri LAB1 at 37° C. settles faster at lower than at higher pH.

Freeze-dried powder of Lactobacillus reuteri LAB1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 1.086, 0.907, 0.909, 0.910 and 0.0.892 after subtracting a blank with ultrapure water. Resulting pH in samples was 5.84, 6.43, 6.83, 7.24 and 7.75, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 37° C. during the experiment. Correction and normalization of OD values were performed as described in Example 1.

The resulting OD₆₀₀ settling time curves at 37° C. in different pH suspensions are depicted in FIG. 12. The normalized OD₆₀₀ time curves of the cell suspensions at various pH indicate that Lactobacillus reuteri LAB1 at 37° C. settles faster at lower than at higher pH.

### Example 13: Settling of Gram-Positive Aerotolerant Anaerobes at 23° C. in Bicarbonate Buffer

The example demonstrates that Lactobacillus reuteri LAB-1 cells suspended in bicarbonate buffer solution settle faster at lower than at higher pH.

Freeze-dried powder of LAB-1 was dissolved in ultrapure water to give a master solution of approximately 10 OD. Subsequently, 15 mL Eppendorf tubes were filled with 1 mL of 1 M carbonate buffer with different Na₂CO₃/NaHCO₃ ratios, 100 mM phosphoric acid where needed, ultrapure water and the corresponding volume of the master solution ad 10 mL, resulting in OD values at λ=600 nm of 0.928, 0.881, 0.903, 0.880, 0.888, 0.855, 0.850 and 0.826 after subtracting a blank with ultrapure water. pH in samples was 6.01, 7.87, 8.15, 8.25, 8.37, 9.49, 9.87 and 10.23, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA).

Measurement of OD₆₀₀ was performed as described in Example 1. The cuvette temperature was controlled at 23° C. during the experiment. Correction and normalization of OD values were performed as described in Example 1.

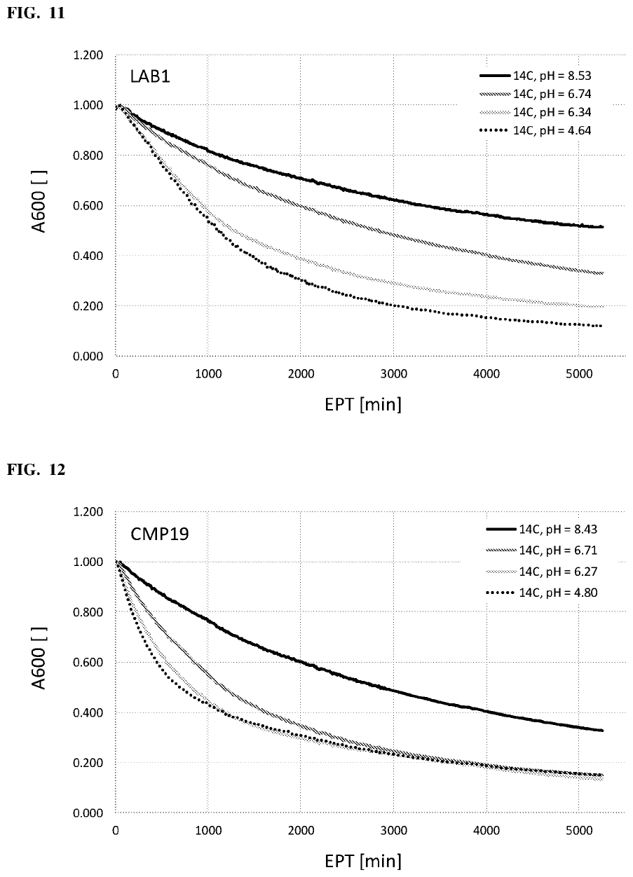

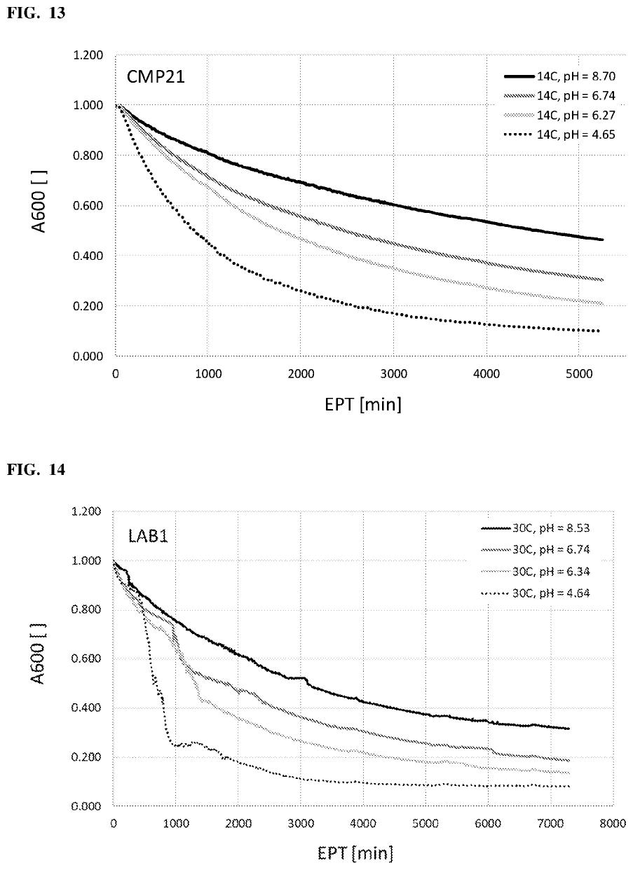

The resulting OD₆₀₀ settling time curves at 23° C. in different pH suspensions are depicted in FIG. 13.

As pH in the cuvettes occasionally changes over time, pH values of the samples in the cuvettes were measured again at the end of the experiment, yielding values of 5.22, 7.88, 7.82, 8.29, 8.37, 9.36, 9.77 and 10.68, respectively.

The normalized OD₆₀₀ time curves of the cell suspensions at various pH indicate that LAB-1 in bicarbonate buffer solutions settle faster at lower than at higher pH.

### Example 14: Settling of Gram-Positive Aerotolerant Anaerobes: Molarity Versus pH

The example demonstrates that settling velocity of LAB-1 depends mainly on the pH value than on the molarity of the suspension.

Freeze-dried powder of LAB-1 was dissolved in ultrapure water to give a master solution of approximately 10 OD. Subsequently, 15 mL Eppendorf tubes were filled with either 1 mL, 0.1 mL or 0.01 mL of 1 M carbonate buffer with different Na₂CO₃/NaHCO₃ ratios, 100 mM phosphoric acid where needed, ultrapure water and the corresponding volume of the master solution ad 10 mL, resulting in OD values of λ=600 nm of 0.826, 0.928, 0.881 (100 mM Na₂CO₃/NaHCO solutions), 0.854 (10 mM Na₂CO₃/NaHCO₃ solutions) and 0.910 (1 mM Na₂CO₃/NaHCO₃ solutions) after subtracting a blank with ultrapure water. pH in samples was 10.23, 7.87, 6.01, 10.60 and 7.75, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA).

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH suspensions are depicted in FIG. 14.

As pH in the cuvettes occasionally changes over time, pH values of the samples in the cuvettes were measured again at the end of the experiment, yielding values of 10.68, 7.88, 5.22, 10.25 and 7.16, respectively.

The normalized OD₆₀₀ time curves of the cell suspensions at various pH indicate that settling velocities of LAB-1 in mono-/disodium carbonate solutions mainly depends on the pH value, and not on the molarity of the solution.

### Example 15: Settling of Yeast Strains at Different pH Values

The example demonstrates that Saccharomyces cerevisiae (from Zenith Yeast Concentrate, AB Biotek, product code: 6400, St. Louis, MO) settles faster at lower than at higher pH.

Zenith Yeast Concentrate (AB Biotek, product code: 6400) was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.926, 0.850, 0.797, 0.832, after subtracting a blank with ultrapure water. Resulting pH in samples was 4.41, 6.32, 6.69 and 8.74, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at different pH suspensions is depicted in FIG. 15. The normalized OD₆₀₀ time curves of the cell suspensions at various pH indicate that S. cerevisiae settles faster at lower than at higher pH.

### Example 16: Settling of Gram-Positive Anaerobes at Different pH Values

This example demonstrates that Bifidobacterium lactis Bl-04® (also known as DGCC2908 and RB 4825, IFF, New York, NY) settles faster at lower than at higher pH.

Freeze-dried powder of Bifidobacterium lactis B1-04® was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.932, 0.868, 0.889, and 0.912 after subtracting a blank with ultrapure water. Resulting pH in samples was 4.65, 6.22, 6.62 and 8.46, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at different pH suspensions is depicted in FIG. 16. The normalized OD₆₀₀ time curves of the cell suspensions at various pH indicate that Bifidobacterium lactis BI-04® settles faster at lower than at higher pH.

### Example 17: Settling of Gram-Positive Anaerobes at Different pH Values

The example demonstrates that Lactobacillus plantarum Lp115 (Danisco, USA) at 23° C. settles faster at lower than at higher pH.

Freeze-dried powder of Lactobacillus plantarum Lp115 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.947, 0.874, 0.924, and 0.860 after subtracting a blank with ultrapure water. Resulting pH in samples was 4.66, 6.22, 6.64 and 8.47, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

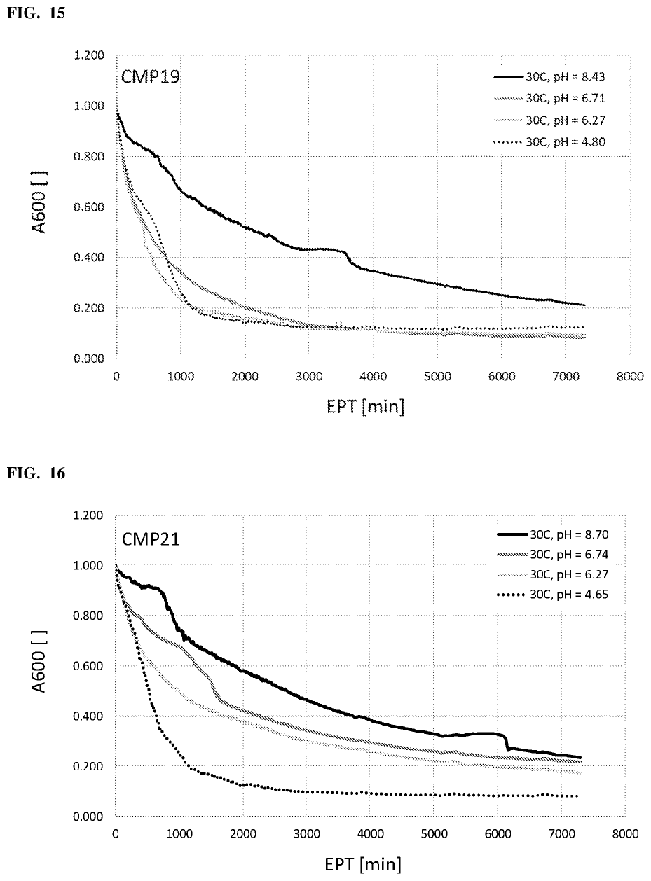

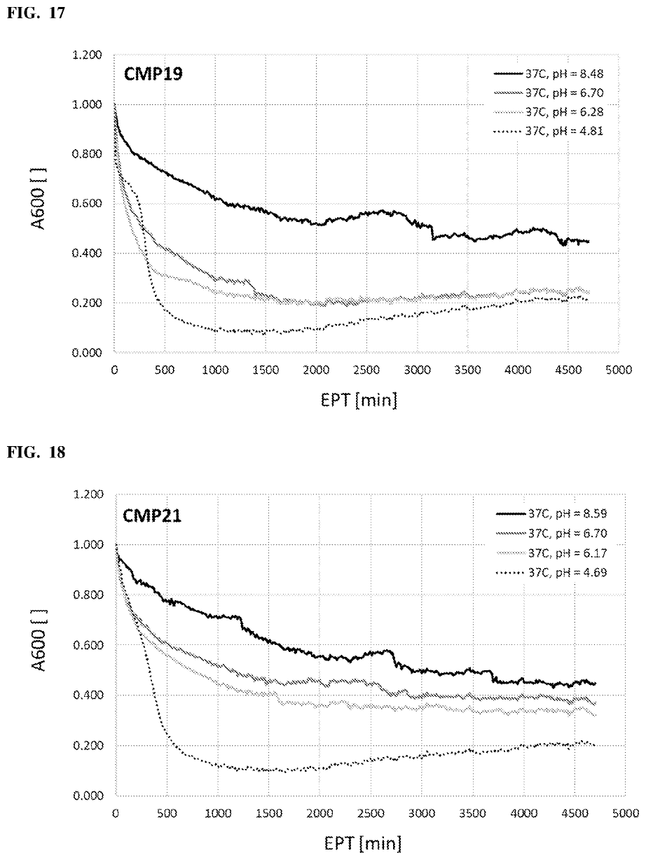

The resulting OD₆₀₀ settling time curves at different pH suspensions is depicted in FIG. 17. The normalized OD₆₀₀ time curves of the cell suspensions at various pH indicate that Lactobacillus plantarum Lp115 settles faster at lower than at higher pH.

### Example 18: Settling of Gram-Positive Aerotolerant Anaerobes at 14° C. at Different pH Values

This example demonstrates that Lactobacillus reuteri LAB1 at 14° C. settles faster at lower than at higher pH.

Freeze-dried powder of Lactobacillus reuteri LAB1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.932, 0.947, 0.919 and 0.933 after subtracting a blank with ultrapure water. Resulting pH in samples was 4.64, 6.34, 6.74 and 8.53, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 14° C. during the experiment. Correction and normalization of OD values were performed as described in Example 1.

The resulting OD₆₀₀ settling time curves at 14° C. in different pH suspensions are depicted in FIG. 28. The normalized OD₆₀₀ time curves of the cell suspensions at various pH indicate that Lactobacillus reuteri LAB1 at 14° C. settles faster at lower than at higher pH.

### Example 19: Settling of Gram-Positive Aerotolerant Anaerobes at 14° C. at Different pH Values

This example demonstrates that Lactobacillus reuteri CMP19 at 14° C. settles faster at lower than at higher pH.

Freeze-dried powder of Lactobacillus reuteri CMP19 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.799, 0.764, 0.744 and 0.759 after subtracting a blank with ultrapure water. Resulting pH in samples was around 4.80, 6.27, 6.71 and 8.43, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 14° C. during the experiment. Correction and normalization of OD values were performed as described in Example 1.

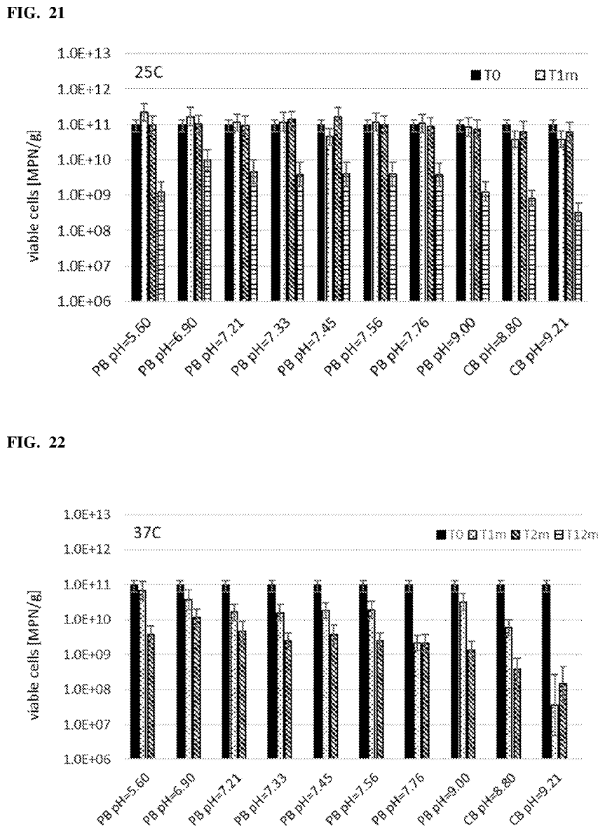

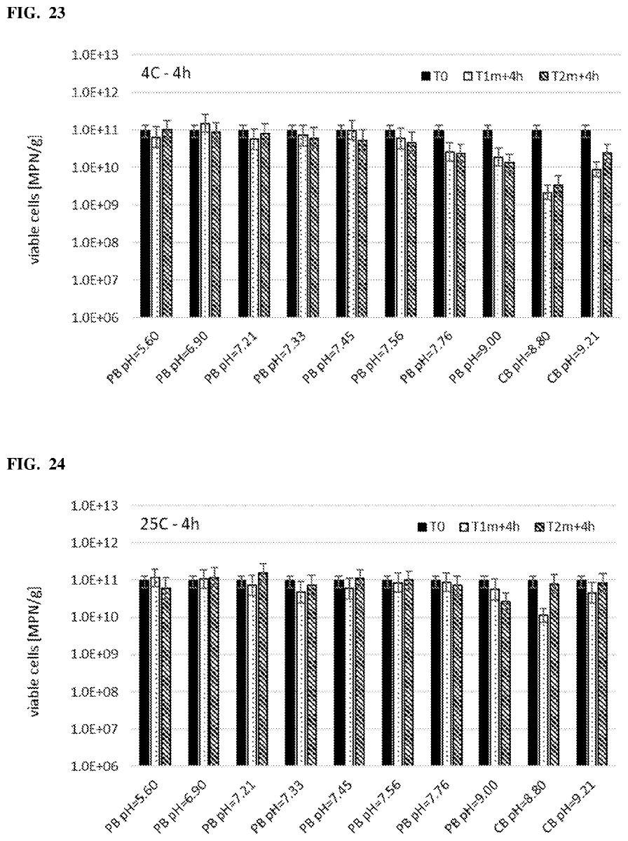

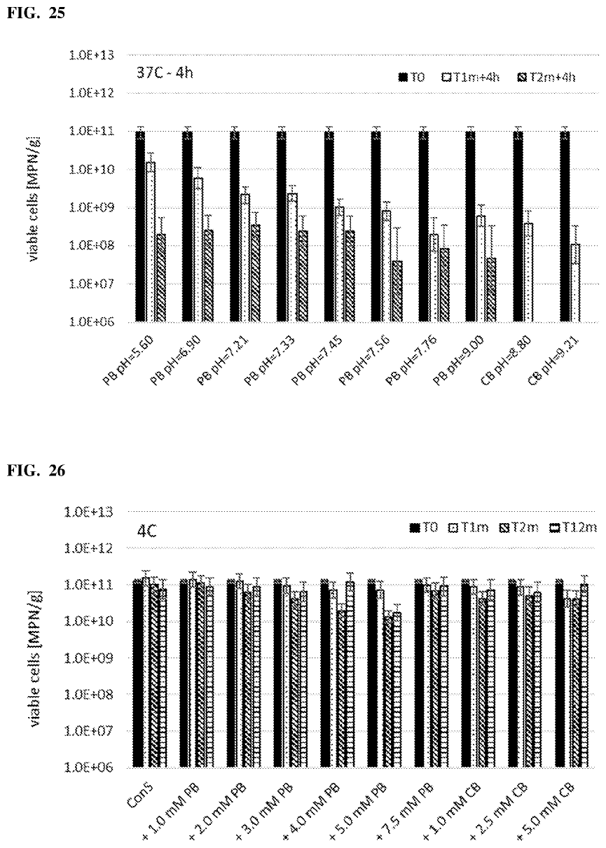

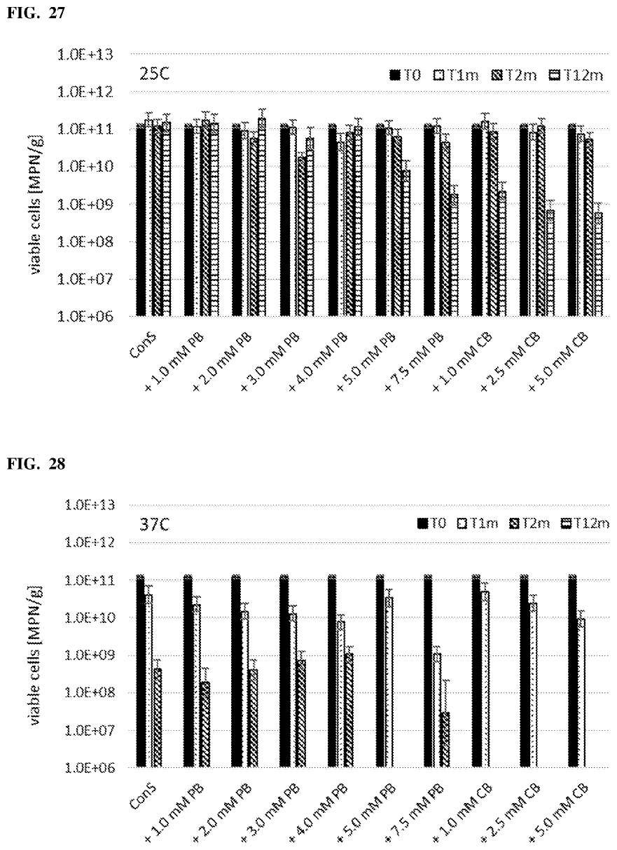

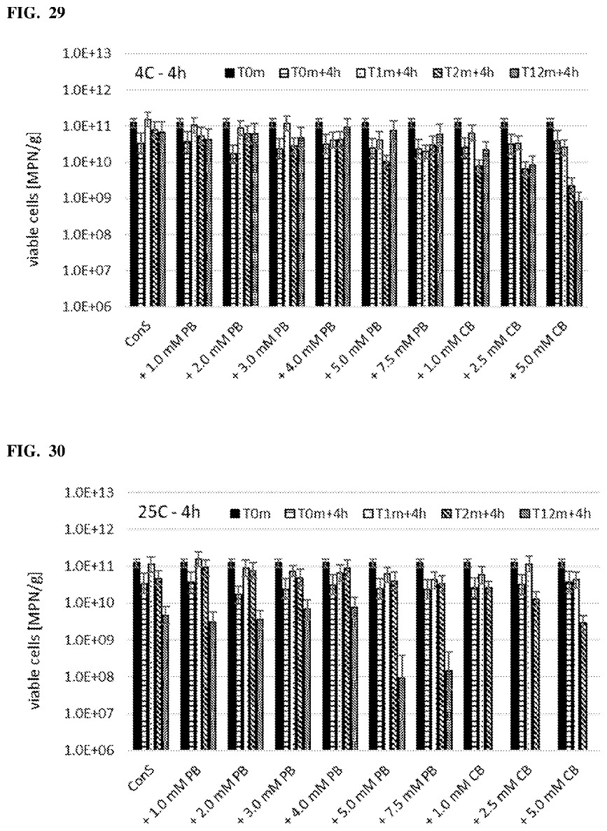

The resulting OD₆₀₀ settling time curves at 14° C. in different pH suspensions are depicted in FIG. 29. The normalized OD₆₀₀ time curves of the cell suspensions at various pH indicate that Lactobacillus reuteri CMP19 at 14° C. settles faster at lower than at higher pH.

### Example 20: Settling of Gram-Positive Aerotolerant Anaerobes at 14° C. at Different pH Values

This example demonstrates that Lactobacillus reuteri CMP21 at 14° C. settles faster at lower than at higher pH.

Freeze-dried powder of Lactobacillus reuteri CMP21 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.870, 0.908, 0.899 and 0.890 after subtracting a blank with ultrapure water. Resulting pH in samples was around 4.65, 6.27, 6.74 and 8.70, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 14° C. during the experiment. Correction and normalization of OD values were performed as described in Example 1.

The resulting OD₆₀₀ settling time curves at 14° C. in different pH suspensions are depicted in FIG. 30. The normalized OD₆₀₀ time curves of the cell suspensions at various pH indicate that Lactobacillus reuteri CMP21 at 14° C. settles faster at lower than at higher pH.

### Example 21; Settling of Gram-Positive Aerotolerant Anaerobes at 30° C. at Different pH Values

This example demonstrates that Lactobacillus reuteri LAB1 at 30° C. settles faster at lower than at higher pH.

Freeze-dried powder of Lactobacillus reuteri L4B1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.901, 0.899, 0.878 and 0.876 after subtracting a blank with ultrapure water. Resulting pH in samples was 4.64, 6.34, 6.74 and 8.53, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 30° C. during the experiment. Correction and normalization of OD values were performed as described in Example 1.

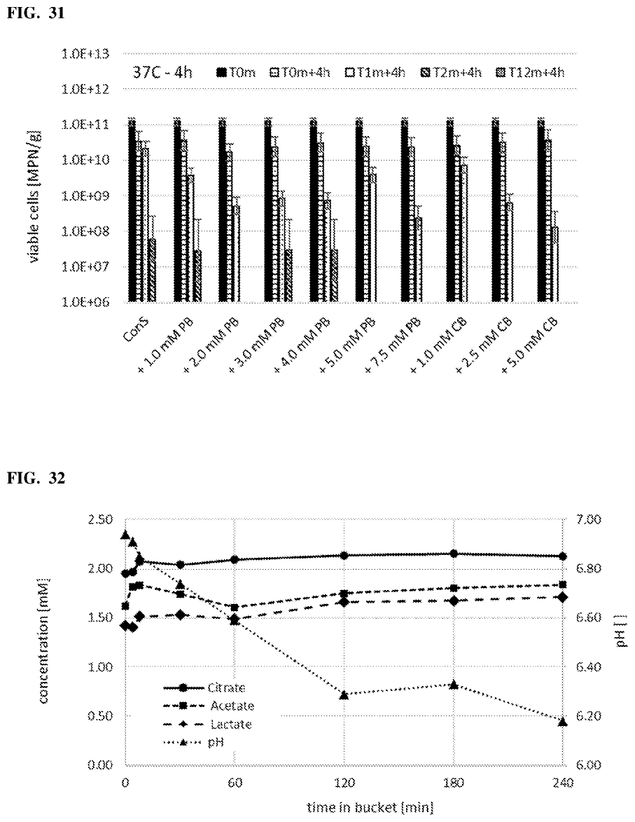

The resulting OD₆₀₀ settling time curves at 30° C. in different pH suspensions are depicted in FIG. 31. The normalized OD₆₀₀ time curves of the cell suspensions at various pH indicate that Lactobacillus reuteri LAB1 at 30° C. settles faster at lower than at higher pH.

### Example 22: Settling of Gram-Positive Aerotolerant Anaerobes at 30° C. at Different pH Values

This example demonstrates that Lactobacillus reuteri CMP19 at 30° C. settles faster at lower than at higher pH.

Freeze-dried powder of Lactobacillus reuteri CMP19 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.719, 0.737, 0.749 and 0.779 after subtracting a blank with ultrapure water. Resulting pH in samples was around 4.80, 6.27, 6.71 and 8.43, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 30° C. during the experiment. Correction and normalization of OD values were performed as described in Example 1.

The resulting OD₆₀₀ settling time curves at 30° C. in different pH suspensions are depicted in FIG. 32. The normalized OD₆₀₀ time curves of the cell suspensions at various pH indicate that Lactobacillus reuteri CMP19 at 30° C. settles faster at lower than at higher pH.

### Example 23: Settling of Gram-Positive Aerotolerant Anaerobes at 30° C. at Different pH Values

This example demonstrates that Lactobacillus reuteri CMP21 at 30° C. settles faster at lower than at higher pH.

Freeze-dried powder of Lactobacillus reuteri CMP21 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.847, 0.889, 0.868 and 0.871 after subtracting a blank with ultrapure water. Resulting pH in samples was around 4.65, 6.27, 6.74 and 8.70, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 30° C. during the experiment. Correction and normalization of OD values were performed as described in Example 1.

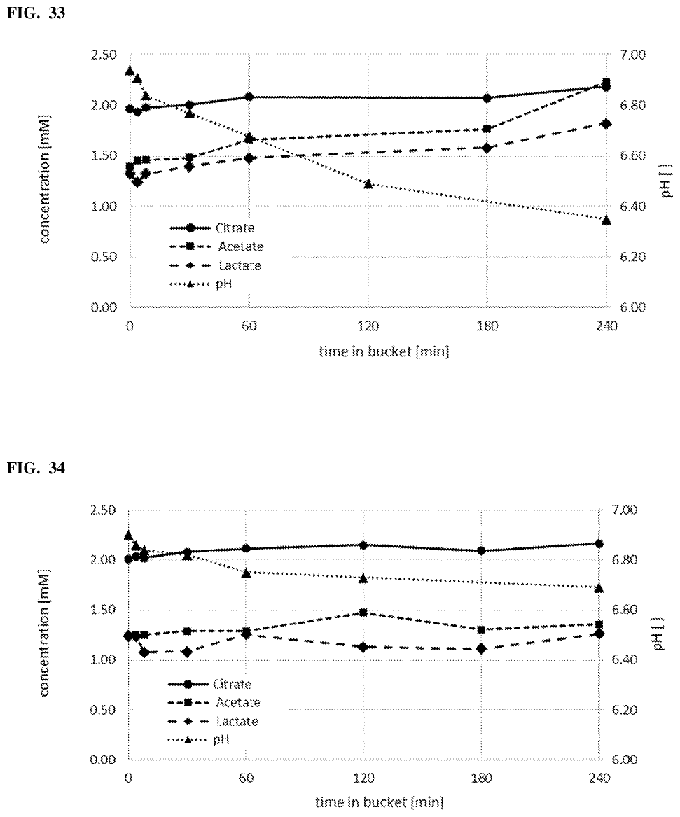

The resulting OD₆₀₀ settling time curves at 30° C. in different pH suspensions are depicted in FIG. 33. The normalized OD₆₀₀ time curves of the cell suspensions at various pH indicate that Lactobacillus reuteri CMP21 at 30° C. settles faster at lower than at higher pH.

### Example 24; Settling of Gram-Positive Aerotolerant Anaerobes at 37° C. at Different pH Values

This example demonstrates that Lactobacillus reuteri CMP19 at 37° C. settles faster at lower than at higher pH.

Freeze-dried powder of Lactobacillus reuteri CMP19 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.877, 0.778, 0.766 and 0.778 after subtracting a blank with ultrapure water. Resulting pH in samples was around 4.81, 6.28, 6.70 and 8.48, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 37° C. during the experiment. Correction and normalization of OD values were performed as described in Example 1.

The resulting OD₆₀₀ settling time curves at 37° C. in different pH suspensions are depicted in FIG. 34. The normalized OD₆₀₀ time curves of the cell suspensions at various pH indicate that Lactobacillus reuteri CMP19 at 37° C. settles faster at lower than at higher pH.

### Example 25: Settling of Gram-Positive Aerotolerant Anaerobes at 37° C. at Different pH Values

This example demonstrates that Lactobacillus reuteri CMP21 at 37° C. settles faster at lower than at higher pH.

Freeze-dried powder of Lactobacillus reuteri CMP21 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.854, 0.868, 0.852 and 0.843 after subtracting a blank with ultrapure water. Resulting pH in samples was around 4.69, 6.17, 6.70 and 8.59, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 37° C. during the experiment. Correction and normalization of OD values were performed as described in Example 1.

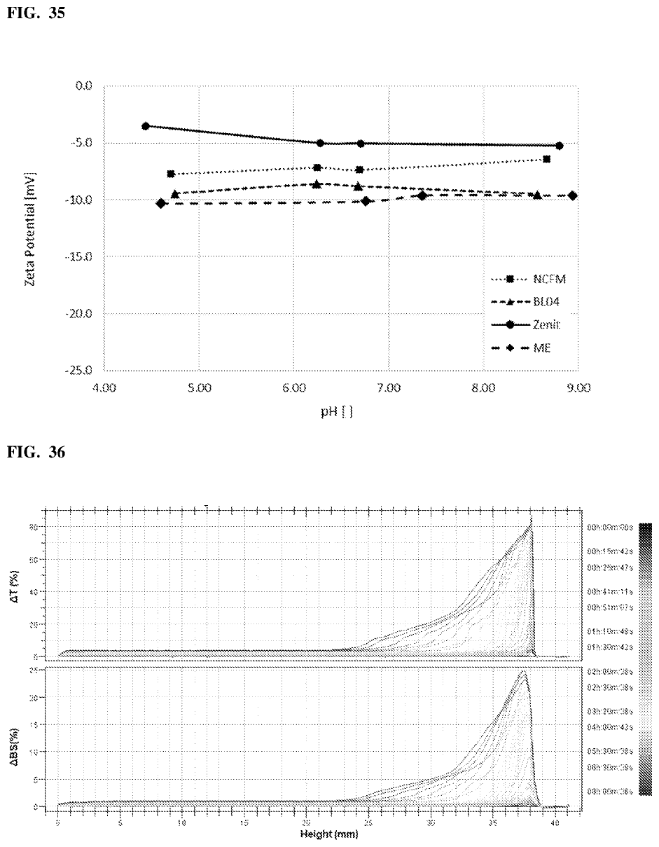

The resulting OD₆₀₀ settling time curves at 37° C. in different pH suspensions are depicted in FIG. 35. The normalized OD₆₀₀ time curves of the cell suspensions at various pH indicate that Lactobacillus reuteri CMP21 at 37° C. settles faster at lower than at higher pH.

### Example 26: Settling of a Three-Strain Consortium of Gram-Positive Aerotolerant Anaerobes at 37° C. at Different pH Values

The example demonstrates that the three-strain Lactobacillus reuteri-containing Consortium-S(ConS) settles faster at lower than at higher pH at 37° C.

Freeze-dried powder of ConS was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.841, 0.819, 0.805 and 0.808 after subtracting a blank with ultrapure water. Resulting pH in samples was 4.68, 6.19, 6.57 and 8.49, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 37° C. during the experiment. Correction and normalization of OD values were performed as described in Example 1.

The resulting OD₆₀₀ settling time curves at 37° C. in different pH suspensions are depicted in FIG. 36. The normalized OD₆₀₀ time curves of the cell suspensions at various pH indicate that ConS at 37° C. settles faster at lower than at higher pH.

### Example 27: Settling of Gram-Positive Aerotolerant Anaerobes at 23° C. at Different pH Values

This example demonstrates that Lactobacillus reuteri LAB-1 at 23° C. settles faster at lower than at higher pH.

Freeze-dried powder of Lactobacillus reuteri LAB-1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.836, 0.827, 0.803, 0.813, 0.823, 0.844 and 0.858 after subtracting a blank with ultrapure water. Resulting pH in samples was 8.42, 7.04, 6.54, 6.05, 5.58, 5.20 and 4.63, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH suspensions are depicted in FIG. 18. The normalized OD₆₀₀ time curves of the cell suspensions at various pH indicate that Lactobacillus reuteri LAB-1 at 23° C. settles faster at lower than at higher pH.

Replicate solutions of the sample solutions were generated following the steps of the protocol. Subsequently, Zeta-potential was measured based on Dynamic Light Scattering (DLS) analysis.

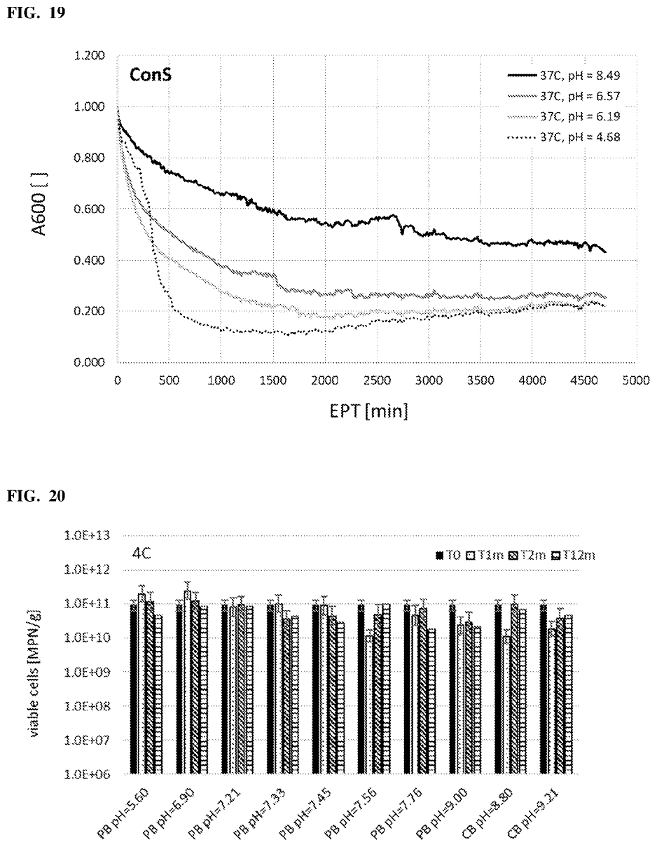

The sample solutions were filled in clear disposable zeta cells (DTS1070, Malvern Instruments Limited, Malvern, U.K.). The cells were transferred into a Zetasizer Nano ZS (ZEN 3600, Malvern Instruments Limited, Malvern, U.K.) equipped with a 4 mW 632.8 nm “red” laser. Analysis was carried out at 23° C. after the sample and cell had equilibrated for 30 s. Instrument set up was as follows: (i) a refractive index of 1.600; (ii) an absorption of 0.100; (iii) a dispersant refractive index of 1.330; (iv) a dispersant viscosity of 0.9308 cP; and (v) a dispersant dielectric constant of 79.3. (Z)-potential analysis was performed using a Doppler laser anemometry function. A Smoluchowski constant F (Ka) of 1.5 was applied. Results are shown in FIG. 19 as the average±standard error of the mean from three independent measurements.

It can be seen that the Zeta-potential of Lactobacillus reuteri LAB-1 at 23° C. in 100 mM of different K₂HPO₄/KH₂PO₄ ratios follows a sigmoid curve with a significant increase in the pH range of 5.5 to 6.5, from approximately −15 mV to approximately −4 mV.

### Example 28; Zeta-Potential of Gram-Positive Aerotolerant Anaerobes at 23° C.

This example demonstrates that the Zeta-Potential of Lactobacillus reuteri CMP-19 increases with increasing pH.

Freeze-dried powder of Lactobacillus reuteri CMP-19 was dissolved in ultrapure water to give a master solution of approximately 10 OD. Subsequently, 15 mL Eppendorf tubes were filled with 1 mL of 25 mM phosphate buffer with different K₂HPO₄/KH₂PO₄ ratios, ultrapure water and 1 mL of the master solution ad 10 mL. Resulting pH in samples was 5.66, 5.85, 5.91, 6.03, 6.19, 6.39, 6.58, 6.77, 6.92, 7.02 and 7.16, respectively, as determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

Measurements of Zeta-potential were carried out as described in Example 27. Results are depicted in FIG. 20.

It can be seen that the Zeta-potential of Lactobacillus reuteri CMP-19 at 23° C. in 2.5 mM of different K₂HPO₄/KH₂PO₄ ratios follows approximately a sigmoid curve with a significant increase in the pH range of 6.7 to 7.2, from approximately −15 to −20 mV to above −10 mV.

### Example 29; Zeta-Potential of Gram-Positive Aerotolerant Anaerobes at 23° C.

This example demonstrates that the Zeta-Potential of Lactobacillus reuteri CMP-21 increases with increasing pH.

Freeze-dried powder of Lactobacillus reuteri CMP-21 was dissolved in ultrapure water to give a master solution of approximately 10 OD. Subsequently, 15 mL Eppendorf tubes were filled with 1 mL of 25 mM phosphate buffer with different K₂HPO₄/KH₂PO₄ ratios, ultrapure water and 1 mL of the master solution ad 10 mL. Resulting pH in samples was 6.10, 6.08, 6.11, 6.23, 6.39, 6.61, 6.80, 6.96, 7.09, 7.20 and 7.35, respectively, as determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

Measurements of Zeta-potential were carried out as described in Example 27. Results are depicted in FIG. 21.

It can be seen that the Zeta-potential of Lactobacillus reuteri CMP-21 at 23° C. in 2.5 mM of different K₂HPO₄/KH₂PO₄ ratios follows approximately a sigmoid curve with a significant increase in the pH range of 6.4 to 7.1, from approximately −20 mV to above −10 mV.

### Example 30: Zeta-Potential of a Three-Strain Consortium of Gram-Positive Aerotolerant Anaerobes at 23° C.

This example demonstrates that the Zeta-Potential of the three-strain Lactobacillus reuteri-containing Consortium-S(ConS) increases with increasing pH.

Freeze-dried powder of ConS was dissolved in ultrapure water to give a master solution of approximately 10 OD. Subsequently, 15 mL Eppendorf tubes were filled with 1 mL of 25 mM phosphate buffer with different K₂HPO₄/KH₂PO₄ ratios, ultrapure water and 1 mL of the master solution ad 10 mL. Resulting pH in samples was 4.57, 4.80, 4.92, 5.09, 5.29, 5.47, 5.63, 7.13, 7.22, and 7.38, respectively, as determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

Measurements of Zeta-potential were carried out as described in Example 27. No distinct populations of the three different strains were detected, supporting the assumption of applying a single fit. Results are depicted in FIG. 22.

It can be seen that the Zeta-potential of the three-strain Lactobacillus reuteri-containing Consortium-S(ConS) at 23° C. in 2.5 mM of different K₂HPO₄/KH₂PO₄ ratios increases in the pH range of around 5.5 to 7.4, from approximately −19 mV to above −12 mV.

### Example 31: Settling of Gram-Positive Aerotolerant Anaerobes at 23° C. in BIS TRIS Buffer

The example demonstrates that Lactobacillus reuteri LAB-1 cells suspended in BIS TRIS buffer solution (also known as, Bis-tris, Bis-tris methane or BTM buffer) settle faster at lower than at higher pH.

Freeze-dried powder of LAB-1 was dissolved in ultrapure water to give a master solution of approximately 10 OD. 1 M BIS TRIS buffer solutions of different pH were generated by adding different amounts of HCl into the 1 M BIS TRIS buffer solutions. Subsequently, 15 mL Eppendorf tubes were filled with 1 mL of 1 M BIS TRIS buffer with different pH, ultrapure water and the corresponding volume of the master solution ad 10 mL, resulting in OD values at λ=600 nm of 0.895, 0.886, 0.897 and 0.868 after subtracting a blank with ultrapure water. pH in samples was 6.98, 6.47, 5.98 and 5.53, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA).

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH suspensions are depicted in FIG. 23. The normalized OD₆₀₀ time curves of the cell suspensions at various pH indicate that LAB-1 in BIS TRIS solutions settle faster at lower than at higher pH.

### Example 32: Settling of Gram-Positive Aerotolerant Anaerobes at 23° C. with Thickeners

This example demonstrates that settling of Lactobacillus reuteri LAB-1 at 23° C. decreases with increasing concentrations of alginate. It also demonstrates that Lactobacillus reuteri LAB-1 at 23° C. in similar concentrations of alginates settles faster at lower than at higher pH.

Freeze-dried powder of Lactobacillus reuteri LAB-1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, different volumes of a 5% (w/v) well-mixed Scogin stock solution (Scogin MV Alginate, IFF, NY), ultrapure water and the corresponding volume of the master solution ad 10 mL and thoroughly mixed, resulting in OD values at k=600 nm of 0.915 (PB+0.1% (w/v) Scogin, pH=5.77), 0.957 (PB+0.1% w/v, pH=6.85), 0.944 (PB+0.3% (w/v) Scogin, pH=5.81), 0.926 (PB+0.3% (w/v) Scogin, pH=6.89), 1.021 (PB+0.6% (w/v) Scogin, pH=5.78) and 0.938 (PB+0.6% (w/v) Scogin, pH=6.85) after subtracting a blank with ultrapure water. pH was determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH/alginate suspensions are depicted in FIG. 24. The normalized OD₆₀₀ time curves of the cell suspensions at various pH indicate that Lactobacillus reuteri LAB-1 at 23° C. settles faster in solutions with lower than higher alginate concentrations. They also demonstrate that at similar alginate concentrations, Lactobacillus reuteri LAB-1 settles faster at lower pH than at higher pH.

### Example 33. Settling of Gram-Positive Aerotolerant Anaerobes at 23° C. with Thickeners

This example demonstrates that settling of Lactobacillus reuteri LAB-1 in a phosphate buffer solution at 23° C. decreases with increasing concentrations of microcrystalline cellulose. It also demonstrates that Lactobacillus reuteri LAB-1 at 23° C. in similar concentrations of microcrystalline cellulose settles faster at lower than at higher pH.

Freeze-dried powder of Lactobacillus reuteri LAB-1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, different volumes of a 5% (w/v) well-mixed microcrystalline cellulose dispersion (Lattice NTC 90, IFF, NY), ultrapure water and the corresponding volume of the master solution ad 10 mL and thoroughly mixed, resulting in OD values at λ=600 nm of 1.493 (PB+0.1% (w/v) NTC 90, pH=5.65), 1.423 (PB+0.1% (w/v) NTC 90, pH=6.87), 2.081 (PB+0.3% (w/v) NTC 90, pH=5.69) and 2.097 (PB+0.3% (w/v) NTC 90, pH=6.89) after subtracting a blank with ultrapure water. pH was determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ 0 was performed as described in Example 1. The cuvette temperature was controlled at 23° C. during the experiment. Correction and normalization of OD values were performed as described in Example 1.

The resulting OD₆₀₀ settling time curves at 23° C. in different pH/alginate suspensions are depicted in FIG. 25. The normalized OD₆₀₀ time curves of the cell suspensions at various pH indicate that Lactobacillus reuteri LAB-1 at 23° C. settles faster in solutions with lower than higher microcrystalline cellulose content. They also demonstrate that in dispersions with similar microcrystalline cellulose content, Lactobacillus reuteri LAB-1 settles faster at lower pH than at higher pH.

### Example 34: Settling of Gram-Positive Aerotolerant Anaerobes at 23° C. with Thickeners

This example demonstrates that settling of Lactobacillus reuteri LAB-1 suspension in phosphate buffer at 23° C. decreases with increasing concentrations of xanthan gum. It also demonstrates that Lactobacillus reuteri LAB-1 at 23° C. in similar concentrations of xanthan gum settles faster at lower than at higher pH.

Freeze-dried powder of Lactobacillus reuteri LAB-1 was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, 7 mL of either ultrapure water, or either a 0.14% (w/v) or a 0.43% (w/v) solution of xanthan gum from Xanthomonas campestris (Sigma-Aldrich G1253, MilliporeSigma, St. Louis, MO) in ultrapure water, the corresponding volume of the master solution and ultrapure water ad 10 mL and thoroughly mixed, resulting in OD values at λ=600 nm of 0.891 (PB, pH=5.65), 0.917 (PB, pH=6.91), 0.986 (PB+0.1% (w/v) xanthan gum, pH=5.55), 0.987 (PB+0.1% (w/v) xanthan gum, pH=6.89), 0.987 (PB+0.3% (w/v) xanthan gum, pH=5.57) and 0.987 (PB+0.3% (w/v) xanthan gum, pH=6.87) after subtracting a blank with ultrapure water. pH was determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at 23° C. in different pH/xanthan gum suspensions are depicted in FIG. 26. The normalized OD₆₀₀ time curves of the cell suspensions at various pH indicate that Lactobacillus reuteri LAB-1 at 23° C. settles faster in solutions with lower than higher xanthan gum concentrations. They also demonstrate that at similar xanthan gum concentrations, Lactobacillus reuteri LAB-1 settles faster at lower pH than at higher pH.

### Example 35: Settling of Gram-Negative Anaerobes at Different pH Values

This example demonstrates that Megasphaera elsdenii settles faster at lower than at higher pH.

Freeze-dried powder of Megasphaera elsdenii 1265 (IFF, New York, NY) was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and the corresponding volume of the master solution ad 10 mL, resulting in OD values at λ=600 nm of 0.727 and 0.717 after subtracting a blank with ultrapure water. Resulting pH values in samples were 6.88 and 8.69, respectively, as determined by a Thermo Scientific Orion 310 PerpHecT LogR pH meter (Thermo Fisher Scientific, Waltham, MA). The pH meter had been calibrated with standards at pH=4.00 and pH=7.00 with pH=4.00 and pH=7.00 Fisher chemical buffer solutions (Thermo Fisher Scientific, Waltham, MA), respectively.

Measurement of OD₆₀₀ was performed as described in Example 1. The cuvette temperature was controlled at 23° C. during the experiment. Correction and normalization of OD values were performed as described in Example 1.

The resulting OD₆₀₀ settling time curves at different pH suspensions is depicted in FIG. 27. The normalized OD₆₀₀ time curves of the cell suspensions at various pH indicate that Megasphaera elsdenii 1265 settles faster at lower than at higher pH.

### Example 36: Determination of Settling Velocity of Optical Fractions in Gram-Positive Anaerobes at Different Temperatures

This example illustrates how settling velocities of different optical fractions were determined for freeze-dried powder of Lactobacillus reuteri LAB1 and how the dependence of their settling velocities on the temperature of the solution can be approximated with an exponential function.

The corrected and normalized OD₆₀₀ settling time curves for different pH suspensions at temperatures of 4° C., 14° C., 23° C., 30° C. and 37° C. from Example 2, Example 6, Example 11, Example 15, and Example 18 were taken as a starting point for the analysis.

Next, the time Δtᵢ required for each curve to reach or fall below values of 0.900, 0.800, 0.700, 0.600, 0.500, 0.400, 0.300, 0.200 and 0.100 were determined and associated with optical density fractions (ODFᵢ) of [0-10%], [10-20%], [20-30%], [30-40%], [40-50%], [50-60%], [60-70%], [70-80%], [80-90%], [90-100%], respectively. All the OD thresholds that were not underrun were assumed to require at least the time of the experiment.

As described previously, for analysis Fisherbrand Cuvettes (#14955125, Thermo Fisher Scientific, Waltham, MA) with a capacity of 4.5 mL were filled with 3.952 mL from the settling solution, thoroughly mixed and subsequently closed with a lid (Fisherbrand cuvette cap square LDPE 19 mm, #14385999, Thermo Fisher Scientific, Waltham, MA). With this setup, the surface of the solution in the cuvette was measured at a filling height of 3.90 cm. The lower limit effecting the light path for the optical density measurement in the Varian Cary 300 Bio UV/Visible spectrophotometer equipped with a temperature controller (Varian Medical Systems, Palo Alto, CA) was determined at a height of 1.80 cm. It was assumed that each optical density fraction ODFᵢ would require to settle 2.10 cm in the cuvette to be detected, resulting in a settling velocity svelᵢ according to

$${svel}_{i} = \frac{2.1 cm}{Δ {t}_{i}}$$

Not all samples were processed, but only samples to be included into pH categories of 5, 6, 7 or 8. Obtained settling velocities for the different samples and their pH classification are listed in FIG. 56.

### Example 37: Determination of Settling Velocity of Optical Fractions in Gram-Positive Anaerobes at Different Temperatures

This example illustrates how settling velocities of different optical fractions were determined for freeze-dried powder of Lactobacillus reuteri CMP/9 and how the dependence of their settling velocities on the temperature of the solution can be approximated with an exponential function.

The corrected and normalized OD₆₀₀ settling time curves for different pH suspensions at temperatures of 4C, 14° C., 23° C., 30° C. and 37° C. from Example 3, Example 7, Example 12, Example 16 and Example 19 were taken as a starting point for the analysis.

Settling velocities off the different optical density fractions (ODF) were determined as described in Example 36 and are shown in FIG. 57.

### Example 38: Determination of Settling Velocity of Optical Fractions in Gram-Positive Anaerobes at Different Temperatures

This example illustrates how settling velocities of different optical fractions were determined for freeze-dried powder of Lactobacillus reuteri CMP21 and how the dependence of their settling velocities on the temperature of the solution can be approximated with an exponential function.

The corrected and normalized OD₆₀₀ settling time curves for different pH suspensions at temperatures of 4C, 14° C., 23° C., 30° C. and 37° C. from Example 4, Example 8, Example 13, Example 17 and Example 20 were taken as a starting point for the analysis.

Settling velocities off the different optical density fractions (ODF) were determined as described in Example 36 and are shown in FIG. 58.

### Example 39: Determination of Settling Velocity of a Three-Strain Consortium of Gram-Positive Aerotolerant Anaerobes at Different Temperatures

This example illustrates how settling velocities of different optical fractions were determined for the three-strain Lactobacillus reuteri-containing Consortium-S(ConS) and how the dependence of their settling velocities on the temperature of the solution can be approximated with an exponential function.

The corrected and normalized OD₆₀₀ settling time curves for different pH suspensions at temperatures of 4° C., 14° C., 23° C., 30° C. and 37° C. from Example 5, Example 14 and Example 21 were taken as a starting point for the analysis.

Settling velocities off the different optical density fractions (ODF) were determined as described in Example 36 and are shown in FIG. 59.

### Example 40: Stability of Viable Cells of Gram-Positive Aerotolerant Anaerobes in Water at Different Temperatures

This example demonstrates that freeze-dried Lactobacillus reuteri LAB1 cells after being dissolved in 2.5 mM phosphate buffer at different temperatures maintain viability for a significant amount of time.

Approximately 0.18 g of Lactobacillus reuteri LAB1 cells freeze-dried in a sucrose and phosphate salt mixture at approximately 5.62 E11 CFU/g were dissolved in ultrapure water and subsequently diluted 1:10 000 in ultrapure water. Next, 1 mL of the diluted cell solution, 1 mL of a 25 mM equimolar phosphate buffer of K₂HPO₄ and KH₂PO₄, as well as 8 mL of ultrapure water were mixed to give a cell solution with initial viable cell count in the range of 1E5-1E6 CFU/mL in 2.5 mM equimolar phosphate buffer.

1.2 mL of the 10 mL cell solution in phosphate buffer was distributed into each of 5 sterile 2 mL Eppendorf tubes, and one Eppendorf tube subsequently transferred in the heat block of a Thermomixer C (Eppendorf, Hamburg, Germany) set at a temperature of 4° C., 14° C., 23° C., 30° C. and 37° C., respectively.

Considering buffer strength of 2.5 mM and the temperature dependence of the disassociation constant of phosphate buffer, the pKa of the phosphate buffer at temperatures of 4° C., 14° C., 23° C., 30° C. and 37° C. is expected to be 7.15, 7.13, 7.11, 7.09 and 7.07, respectively (worldwideweb.reachdevices.com/Protein/BiologicalBuffers.html, accessed: 2/22/2022). For equimolar concentration of 1.25 mM K²HPO₄ and 1.25 mM KH₂PO₄ the resulting pH of the solution at 4° C., 14° C., 23° C., 30° C. and 37° C. is expected to be 7.15, 7.13, 7.11, 7.09 and 7.07, respectively.

At start of the experiment, as well as after 4 h, 8 h, 24 h and 48 h, from each of the five Eppendorf tubes, 40 μL of sample solution was withdrawn and transferred in a 384-well polypropylene and ECHO-qualified source plate (Beckman Coulter, Brea, CA), “undilute” sample. An additional 100 μL sample was withdrawn from each Eppendorf tube, diluted in 900 μL of 2.5 mM K2HPO4/KH2PO4 phosphate buffer, and again 40 μL of this “1:10 dilute” sample transferred into the ECHO source plate.

Next, 25 nL and 2.5 nL of the well-mixed “undilute” sample were transferred with an Echo 550 liquid handler (Beckman Coulter, Brea, CA) into 12 wells each of a 384-well polypropylene and ECHO-qualified target plate with clear bottom (Beckman Coulter, Brea, CA), respectively. In addition, 2.5 nL of the “1:10 dilute” sample were transferred into each of 8 wells of the ECHO target plate. The ECHO target plate had been previously filled with 30 μL of Difco Lactobacilli MRS medium (Thermo Fisher Scientific, Waltham, MA) augmented with 0.05% cysteine.

Next, the inoculated ECHO target plate was transferred into a GasPak EZ Standard Incubation Container (Becton Dickinson, Franklin Lakes, NJ) filled with 2 BD GasPak™ EZ Anaerobe Container System Sachets with Indicator (Becton Dickinson, Franklin Lakes, NJ), and the box stored in an Infors HT Multitron incubator (Infors HT, Bottmingen, Switzerland) set at 37° C. for at least 48 h. After storage, optical density of the wells was read with a TECAN Infinite 200 Pro plate reader (TECAN, Männedorf, Switzerland) at λ=600 nm to indicate if at least one cell had been transferred and multiplied in the well (typically resulting in an optical density of >0.195), or not. MRS medium augmented with cysteine gave optical density readings of ≈0.120-0.190.

From the reading, for each temperature sample a list was compiled indicating how many wells of the three transfer regimes (25 nL from “non-dilute”, 2.5 nL from “non-dilute”, 2.5 nL from “1:10 dilute”) showed growth, as compared to the total number of wells inoculated. Based on this list, the viable cell concentration in the storage solution was determined applying “most probable number (MPN)” analysis as described by Jarvis et al. (Journal of Applied Microbiology 109: 1660-1667, 2010). Observed viable cell numbers are provided in FIG. 60.

### Example 41: Stability of Viable Cells of Gram-Positive Aerotolerant Anaerobes in Water at Different Temperatures

This example demonstrates that freeze-dried Lactobacillus reuteri CMP19 cells after being dissolved in 2.5 mM phosphate buffer at different temperatures maintain viability for a significant amount of time.

Approximately 0.16 g of Lactobacillus reuteri CMP19 cells freeze-dried in a sucrose and phosphate salt mixture at approximately 6.58 E10 CFU/g were dissolved in ultrapure water and subsequently diluted 1:1 000 in ultrapure water.

Stability testing and MPN determination was carried out as described in Example 40. Wells with cell growth resulted in optical density readings >0.195. Observed viable cell numbers are provided in FIG. 61.

### Example 42: Stability of Gram-Positive Aerotolerant Anaerobes in Water at Different Temperatures

This example demonstrates that freeze-dried Lactobacillus reuteri CMP21 cells after being dissolved in 2.5 mM phosphate buffer at different temperatures maintain viability for a significant amount of time.

Approximately 0.31 g of Lactobacillus reuteri CMP21 cells freeze-dried in a sucrose and phosphate salt mixture at approximately 3.26 E11 CFU/g were dissolved in ultrapure water and subsequently diluted 1:10 000 in ultrapure water.

Stability testing and MPN determination was carried out as described in Example 40 Example. Wells with cell growth resulted in optical density readings >0.195. Observed viable cell numbers are provided in FIG. 62.

### Example 43: Stability of Viable Cells of a Consortium of Gram-Positive Aerotolerant Anaerobes in Water at Different Temperatures

This example demonstrates that freeze-dried cells of the three-strain Lactobacillus reuteri consortium ConS after being dissolved in 2.5 mM phosphate buffer at different temperatures maintain viability for a significant amount of time.

Approximately 0.65 g of cells of the three-strain Lactobacillus reuteri consortium ConS freeze-dried in a sucrose and phosphate salt mixture at approximately 1.55 E12 CFU/g were dissolved in ultrapure water and subsequently diluted 1:100 000 in ultrapure water.

Stability testing and MPN determination was carried out as described in Example 40. Well with cell growth resulted in optical density readings >0.195. Observed viable cell numbers are provided in FIG. 63.

### Example 44; Assessing Storage Stability of Formulated Gram-Positive Aerotolerant Anaerobes

This example demonstrates that freeze-dried cells of the three-strain Lactobacillus reuteri consortium in stabilizer are stable/maintain a significant degree of viability if stored with different ratios of mono- and dipotassium phosphate or mono- and disodium bicarbonate.

For this purpose, ca. 0.20 g of freeze-dried powder, comprised of cells of the three Lactobacillus reuteri strain LAB1, CMP19 and CMP21, mono- and dipotassium phosphate and sucrose, were blended with different ratios of mono- and dipotassium phosphate according to Table 1, and stored in 50 mL conical sterile Polypropylene centrifuge tubes (Nunc 50 mL, ThermoFisher, Waltham, MA) that further were enclosed in aluminum-coated pouches to prevent oxygen and moisture exchange. In addition, a control sample “S1” was not blended. Of each sample series (A-H and W-Z) 5 tubes were filled, and exact amounts of ConS added per vial determined with a Mettler Toledo XS104 analytical balance (Columbus, OH).

TABLE 1
Blended ConS formulations with composition of different
sample series.

| | ConS | KH₂PO₄ | K₂HPO₄ | NaHCO₃ | Na₂CO₃ |
|---|---|---|---|---|---|
| sample | [g] | [g] | [g] | [g] | [g] |
| A | ≈0.20 | 1.74 | 0.00 | — | — |
| B | ≈0.20 | 1.17 | 0.73 | — | — |
| C | ≈0.20 | 0.87 | 1.11 | — | — |
| D | ≈0.20 | 0.75 | 1.28 | — | — |
| E | ≈0.20 | 0.64 | 1.41 | — | — |
| F | ≈0.20 | 0.54 | 1.53 | — | — |
| G | ≈0.20 | 0.38 | 1.75 | — | — |
| H | ≈0.20 | 0.00 | 2.23 | — | — |
| W | ≈0.20 | — | — | 1.08 | 0.00 |
| X | ≈0.20 | — | — | 1.00 | 0.10 |
| Y | ≈0.20 | — | — | 0.93 | 0.19 |
| Z | ≈0.20 | — | — | 0.00 | 1.36 |
| S1 | ≈0.20 | — | — | — | — |

Before storage of the material (time point T₀), and after 1 month (T₁ₘ), 2 months (T₂ₘ) and 12 months (T12m) of storage of tubes at either 4° C., 25° C., and 37° C. in either a VWR® Chromatography Refrigerator with Glass Doors (Radnor, PA) for the V° C. samples, or a static Innova 4230 shakers (New Brunswick Scientific, NJ, USA) for the 25° C. and 37° C. samples, respectively, the tubes were removed from storage and its content dissolved in 40 ml of ultrapure water tempered at 14° C. and subsequently the solution well mixed. In detail, the T₁ₘ, T₂ₘ and T₁₂ₘ samples were stored for exactly 29, 56 and 393 days at the described conditions, respectively.

The dissolved sample material was diluted up to 1:100 000 000 in 1:10 dilution steps (100 μl into 900 μl) with Difco Lactobacilli MRS medium (Thermo Fisher Scientific, Waltham, MA) that had been augmented with 0.05% cysteine. From each of the 1:10, 1:100, 1:1000 and 1:10 000 dilutions, 40 μL each were transferred in a 384-well polypropylene and ECHO-qualified source plate (Beckman Coulter, Brea, CA).

Next, per sample and dilution 2.5 nL each were transferred with an Echo 550 liquid handler (Beckman Coulter, Brea, CA) into 16 wells of a 384-well polypropylene and ECHO-qualified target plate with clear bottom (Beckman Coulter, Brea, CA), respectively. The ECHO target plate had been previously filled with 30 μL of Difco Lactobacilli MRS medium (Thermo Fisher Scientific, Waltham, MA) augmented with 0.05% cysteine.

Next, the inoculated ECHO target plate was transferred into a GasPak EZ Standard Incubation Container (Becton Dickinson, Franklin Lakes, NJ) filled with 2 BD GasPak™ EZ Anaerobe Container System Sachets with Indicator (Becton Dickinson, Franklin Lakes, NJ), and the box stored in an Infors HT Multitron incubator (Infors HT, Bottmingen, Switzerland) set at 37° C. for at least 48 h. After storage, optical density of the wells was read with a TECAN Infinite 200 Pro plate reader (TECAN, Männedorf, Switzerland) at λ=600 nm to indicate if at least one cell had been transferred and multiplied in the well (typically resulting in an optical density of >0.195), or not. MRS medium augmented with cysteine gave optical density readings of ≈0.120-0.190.

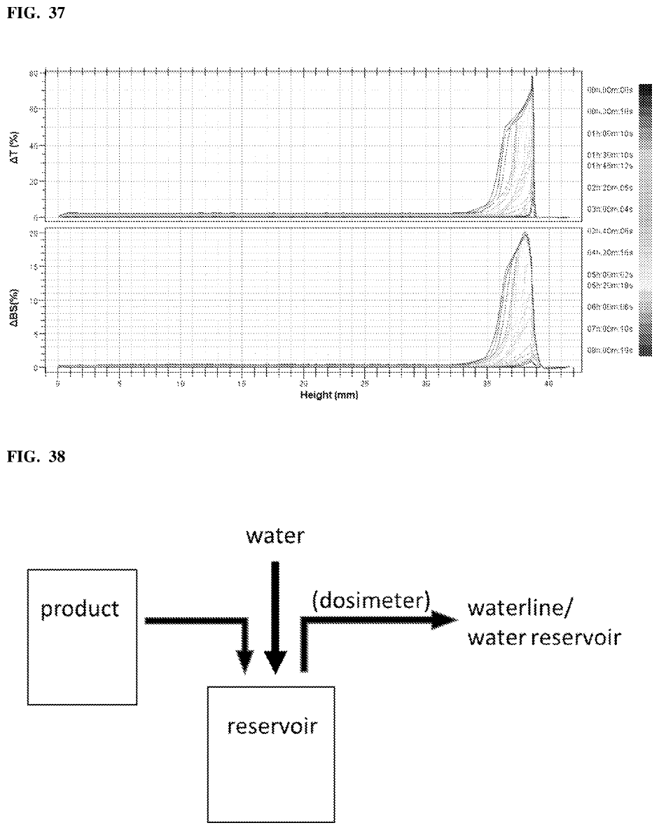

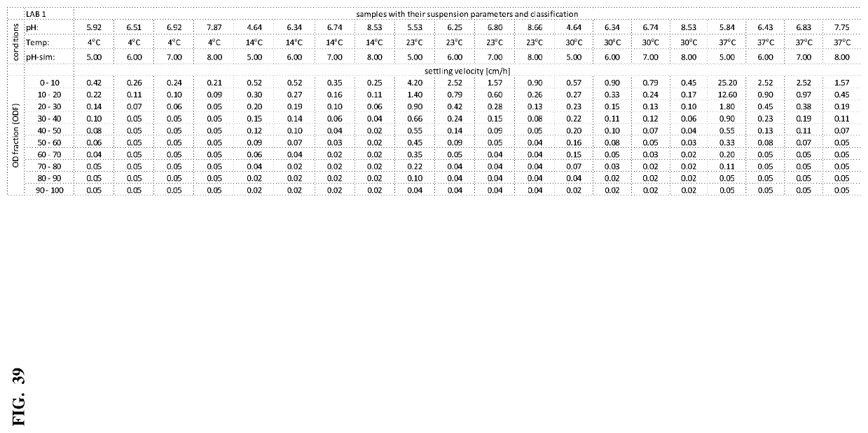

From the reading, for each sample a list was compiled indicating how many wells of each of the four dilutions showed growth, as compared to the total number of wells inoculated. Based on this list, the viable cell concentration in the storage solution was determined applying “most probable number (MPN)” analysis as described by Jarvis et al. (Journal of Applied Microbiology 109: 1660-1667, 2010). Observed viable cell numbers for the rehydrated sample material previously stored at 4° C., 25° C., and 37° C. are depicted in FIG. 37, FIG. 38 and FIG. 39, respectively.

### Example 45: Assessing Stability of Formulated Gram-Positive Aerotolerant Anaerobes in Water after Storage and Resuspension

This example demonstrates that cells of a three-strain Lactobacillus reuteri consortium processed and stored with different ratios of mono- and dipotassium phosphate or mono- and disodium bicarbonate are stable/maintain a significant degree of viability if rehydrated in water.

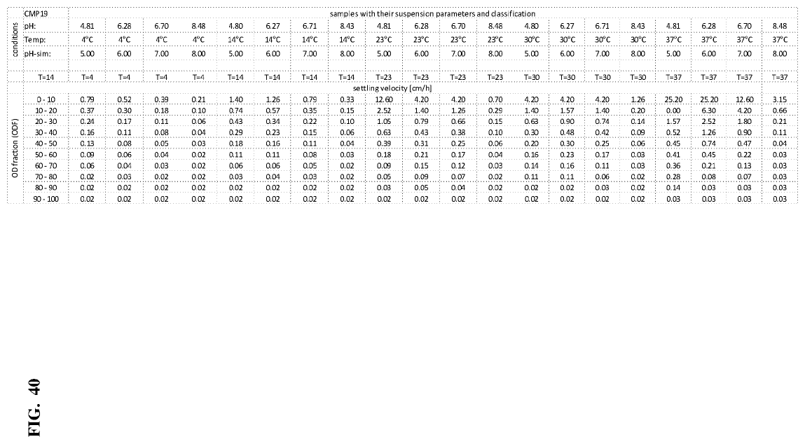

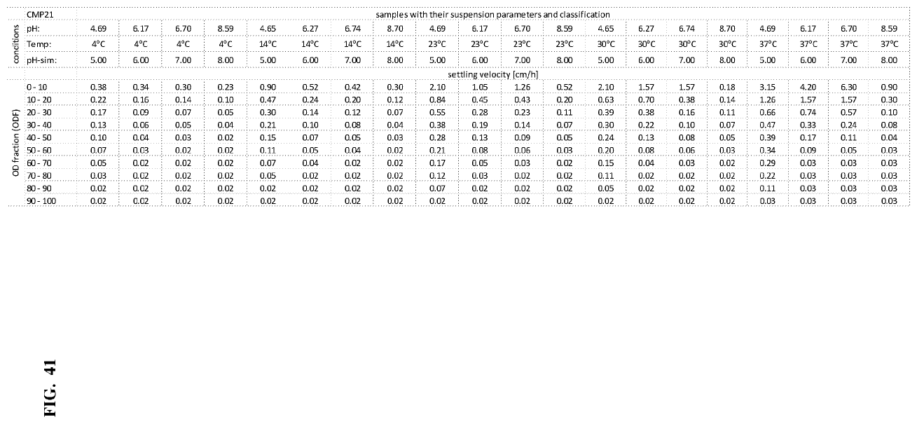

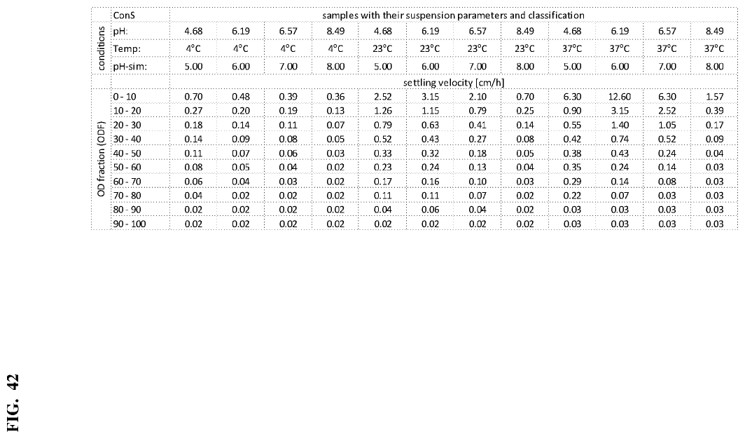

For this purpose, the formula of freeze-dried powder from Example 45, comprised of cells of the three Lactobacillus reuteri strain LAB1, CMP19 and CMP21, mono- and dipotassium phosphate and sucrose, and re-suspended in ultrapure water, was stored at 14° C. for 4 h, and subsequently viability analyzed applying the MPN method as described in Example 45. T0 samples were not stored for 4 h, but viability immediately measured after rehydration. Observed viable cell numbers for the rehydrated sample material previously stored at 4° C., 25° C., and 37° C. and subsequently kept for 4 h in 14° C. water are depicted in FIG. 40, FIG. 41 and FIG. 42, respectively.

PH of the solutions immediately after rehydration (“T[X]”, Example 44) or after 4 h dissolved in water (“T[X]+4 h”), where [X] designates the respective time point, were measured and are provided in buffer (different ratios of mono- and dipotassium phosphate or mono- and disodium bicarbonate) (i) reduces the resulting change of pH from the blended products immediately after rehydration, especially if the pH is around the pKa value of the blended buffer, and (ii) reduces the resulting pH change during the 4 h incubation in water, especially if the pH is around the pKa value of the blended buffer.

PH was determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

Discounting the single set of T₀ samples pH measurements that seemingly were carried out with an ill-calibrated pH meter, the measurements demonstrate that additional blending the freeze-dried powder with buffer (different ratios of mono- and dipotassium phosphate or mono- and disodium bicarbonate) (i) reduces the resulting change of pH from the blended products immediately after rehydration, especially if the pH is around the pKa value of the blended buffer, and (ii) reduces the resulting pH change during the 4 h incubation in water, especially if the pH is around the pKa value of the blended buffer (Table 2).

TABLE 2
pH measured in rehydrated samples either immediately
after resuspension “T[X]”, and/or after
incubation for 4 h in water at 14° C., “T[X + 4 h]”.

| storage | | | | | | | |
|---|---|---|---|---|---|---|---|
| temp | | sample | T₀* | T_{1 m+4 h} | T_{2 m} | T_{12 m} | T_{12 m+4 h} |
| 37 | C. | A | 4.67 | 5.75 | 5.18 | 4.69 | 4.68 |
| | | B | 6.29 | n.d. | 6.69 | 6.34 | 6.34 |
| | | C | 6.64 | 6.96 | 7.00 | 6.72 | 6.74 |
| | | D | 6.80 | 7.43 | 7.14 | 6.80 | 6.79 |
| | | E | 6.91 | 7.55 | 7.28 | 6.95 | 6.95 |
| | | F | 7.03 | 7.63 | 7.41 | 7.09 | 7.09 |
| | | G | 7.25 | 7.60 | 7.65 | 7.24 | 7.20 |
| | | H | 8.57 | 8.64 | 8.87 | 8.40 | 8.42 |
| | | W | 7.84 | 9.10 | 8.28 | 8.23 | 8.17 |
| | | X | 8.44 | 9.17 | 8.89 | 8.65 | 8.73 |
| 25 | C. | A | 4.67 | 5.64 | 5.02 | 4.75 | 4.69 |
| | | B | 6.29 | 6.90 | 6.73 | 6.34 | 6.33 |
| | | C | 6.64 | 7.16 | 7.10 | 6.64 | 6.66 |
| | | D | 6.80 | 7.33 | 7.26 | 6.81 | 6.80 |
| | | E | 6.91 | 7.47 | 7.62 | 6.92 | 6.93 |
| | | F | 7.03 | 7.43 | 7.80 | 7.03 | 7.06 |
| | | G | 7.25 | 7.11 | 8.01 | 7.26 | 7.26 |
| | | H | 8.57 | 8.92 | 9.07 | 8.50 | 8.51 |
| | | W | 7.84 | 8.86 | 8.71 | 8.16 | 8.14 |
| | | X | 8.44 | 9.16 | 9.21 | 8.64 | 8.69 |
| 4 | C. | A | 4.67 | 5.56 | 5.30 | 4.71 | 4.69 |
| | | B | 6.29 | 6.80 | n.d. | 6.32 | 6.31 |
| | | C | 6.64 | 7.21 | 7.12 | 6.66 | 6.65 |
| | | D | 6.80 | 6.70 | 7.48 | 6.80 | 6.82 |
| | | E | 6.91 | 7.36 | 7.34 | 6.94 | 6.93 |
| | | F | 7.03 | 7.30 | 7.60 | 7.09 | 7.05 |
| | | G | 7.25 | 7.67 | 7.96 | 7.28 | 7.28 |
| | | H | 8.57 | 8.87 | 9.12 | 8.59 | 8.61 |
| | | W | 7.84 | 8.84 | 9.03 | 8.52 | 8.10 |
| | | X | 8.44 | 9.05 | 9.27 | 8.82 | 8.73 |
| 37 | C. | S1 | 5.94 | n.d. | n.d. | 6.47^{&} | 6.41^{&} |
| 25 | C. | S1 | 5.94 | 7.05^{#} | n.d. | 6.78^{&} | 6.44^{&} |
| 4 | C. | S1 | 5.94 | 6.33^{#} | n.d. | 6.42^{&} | 6.21^{&} |

*measurements in T₀ were only carried out for one sample set, as no previous storage at three different temperatures of the same sample composition was required
^{#}pH measured immediately after dissolving, not after 4 h in water
^{&}S1 samples were stored for 29 d less than the other samples in the respective conditions

### Example 46: Assessing Storage Stability of Formulated Gram-Positive Aerotolerant Anaerobes

This example demonstrates that freeze-dried cells of the three-strain Lactobacillus reuteri consortium in stabilizer are stable/maintain a significant degree of viability if stored with different amounts of mono- and dipotassium phosphate or different amounts of monosodium bicarbonate.

For this purpose, ca. 0.20 g of freeze-dried powder, comprised of cells of the three Lactobacillus reuteri strain LAB1, CMP19 and CMP21, mono- and dipotassium phosphate and sucrose, were mixed with different ratios of mono- and dipotassium phosphate or different amounts of monosodium bicarbonate according to Table 3, and stored in 50 mL conical sterile Polypropylene centrifuge tubes (Nunc 50 mL, ThermoFisher, Waltham, MA) that further were enclosed in aluminum pouches to prevent oxygen and moisture exchange. Of each sample series (A-H and W-Z) 5 tubes were filled, and the exact amounts of ConS added per vial determined with a Mettler Toledo XS104 analytical balance (Columbus, OH).

TABLE 3
Blended ConS formulations of samples “TA2002”.

| | ConS | KH₂PO₄ | K₂HPO₄ | NaHCO₃ | Na₂CO₃ |
|---|---|---|---|---|---|
| sample | [g] | [g] | [g] | [g] | [g] |
| A | ≈0.20 | 0.00 | 0.00 | — | — |
| B | ≈0.20 | 0.35 | 0.45 | — | — |
| C | ≈0.20 | 0.70 | 0.89 | — | — |
| D | ≈0.20 | 1.05 | 1.34 | — | — |
| E | ≈0.20 | 1.39 | 1.78 | — | — |
| F | ≈0.20 | 1.74 | 2.23 | — | — |
| G | ≈0.20 | 2.61 | 3.34 | — | — |
| H | ≈0.20 | 3.48 | 4.46 | — | — |
| W | ≈0.20 | — | — | 0.43 | — |
| X | ≈0.20 | — | — | 1.08 | — |
| Y | ≈0.20 | — | — | 2.15 | — |
| Z | ≈0.20 | — | — | 4.30 | — |

Before storage of the material (time point T₀), and after 1 month (T₁ₘ), 2 months (T₂ₘ) and 12 months (T₁₂ₘ) storage of tubes at either 4° C., 25° C., and 37° C. in either a VW R® Chromatography Refrigerator with Glass Doors (Radnor, PA) for the 4° C. samples, or a static Innova 4230 shakers (New Brunswick Scientific, NJ, USA) for the 25° C. and 37° C. samples, respectively, the tubes were removed from storage and their content dissolved in 40 ml of ultrapure water tempered at 14° C. and subsequently the solution well mixed. In detail, the T1m, T₂ₘ and T₁₂ₘ samples were stored for exactly 38, 66 and 395 days at the described conditions, respectively.

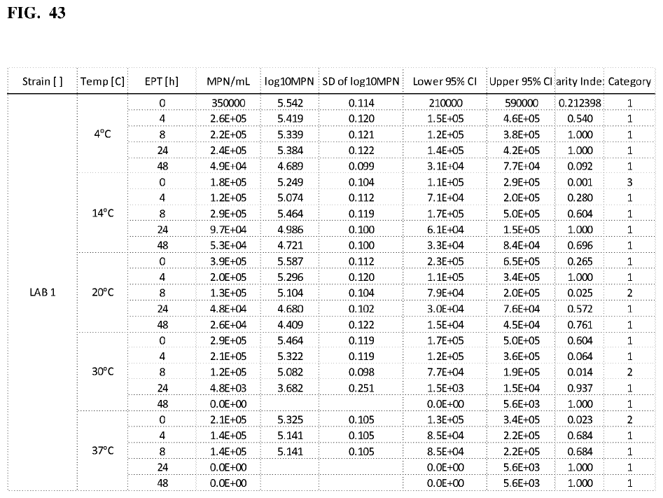

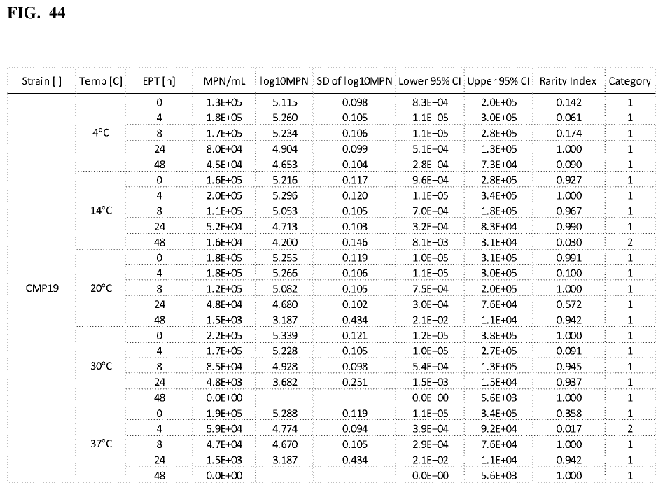

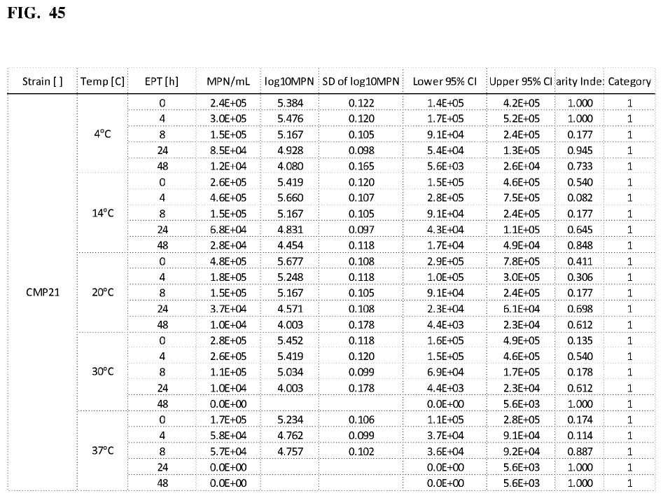

Viability of the sample material was analyzed applying the MPN method as described in Example 45. Observed viable cell numbers for the rehydrated sample material previously stored at 4° C., 25° C., and 37° C. immediately after re-hydration are depicted in FIG. 43, FIG. 44 and FIG. 45, respectively.

### Example 47: Assessing Storage Stability of Formulated Gram-Positive Aerotolerant Anaerobes in Water after Storage and Resuspension

This example demonstrates that cells of a three-strain Lactobacillus reuteri consortium processed and stored with different amounts of mono- and dipotassium phosphate, or with different amounts monosodium bicarbonate are stable/maintain a significant degree of viability if rehydrated in water.

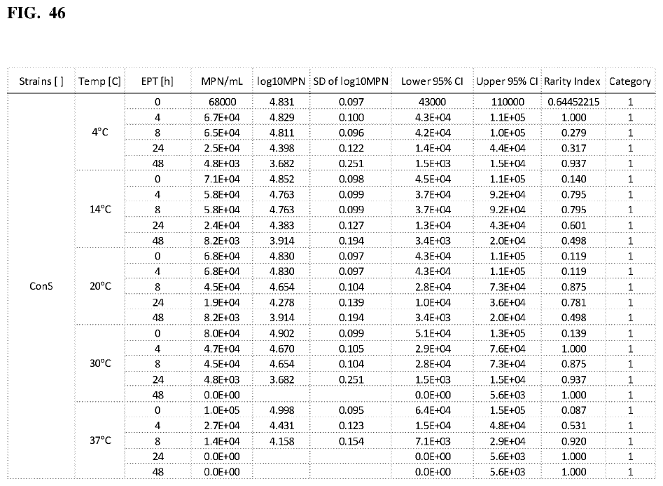

For this purpose, the formula of freeze-dried powder from Example 46, comprised of cells of the three Lactobacillus reuteri strain LAB1, CMP19 and CMP21, either different amounts of mono- and dipotassium phosphate and sucrose, or similar amount of mono- and dipotassium but additionally different amounts of monosodium bicarbonate, were re-suspended in ultrapure water, stored at 14° C. for 4 h, and subsequently viability analyzed applying the MPN method as described in Example 45. Observed viable cell numbers for the rehydrated sample material previously stored at 4° C., 25° C., and 37° C. and then kept at 4 h in water are depicted in FIG. 46, FIG. 47 and FIG. 48, respectively.

PH of the solutions immediately after rehydration (“T[X]”, Example 44) or after 4 h dissolved in water (“T[X]+4 h”), where [X] designates the respective time point, were measured and are provided in Table 4. PH was determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

The measurements demonstrate that additional blending the freeze-dried powder with buffer (different amounts of mono- and dipotassium phosphate or mono- and disodium bicarbonate) (i) reduces the resulting change of pH from the blended products immediately after rehydration, especially if the pH is around the pKa value of the blended buffer, and (ii) reduces the resulting pH change during the 4 h incubation in water, especially if the pH is around the pKa value of the blended buffer.

TABLE 4
pH measured in rehydrated samples either immediately
after resuspension “T[X]”, and/or after
incubation for 4 h in water at 14° C., “T[X + 4 h]”.

| storage | | | | | | | | |
|---|---|---|---|---|---|---|---|---|
| temp | | sample | T_{0+4 h}* | T_{1 m+4 h} | T_{2 m} | T_{2 m+4 h} | T_{12 m} | T_{12 m+4 h} |
| 37 | C. | A | 5.72 | n.d. | 6.39 | 4.62 | 6.55 | 6.33 |
| | | B | 6.75 | 7.04 | 6.79 | 6.47 | 6.72 | 6.67 |
| | | C | 6.74 | 6.99 | 6.73 | 6.46 | 6.68 | 6.64 |
| | | D | 6.70 | 7.02 | 6.73 | 6.52 | 6.64 | 6.57 |
| | | E | 6.69 | 6.95 | 6.70 | 6.56 | 6.60 | 6.56 |
| | | F | 6.62 | 6.84 | 6.70 | 6.63 | 6.59 | 6.53 |
| | | G | 6.64 | 6.92 | 6.69 | 6.68 | 6.57 | 6.54 |
| | | H | 6.63 | 8.79 | 8.19 | 8.05 | 8.06 | 8.07 |
| | | W | 8.06 | 8.76 | 8.35 | 8.35 | 8.10 | 8.12 |
| | | X | 8.27 | 9.15 | 8.33 | 8.55 | 8.07 | 8.05 |
| 25 | C. | A | 5.72 | 5.81 | 6.30 | n.d. | 6.82 | 5.98 |
| | | B | 6.75 | 6.94 | 6.78 | 6.35 | 6.77 | 6.71 |
| | | C | 6.74 | 6.93 | 6.78 | 6.63 | 6.65 | 6.59 |
| | | D | 6.70 | 6.93 | 6.72 | 6.74 | 6.67 | 6.61 |
| | | E | 6.69 | 6.93 | 6.73 | 6.73 | 6.61 | 6.62 |
| | | F | 6.62 | 6.88 | 6.69 | 7.29 | 6.60 | 6.60 |
| | | G | 6.64 | 6.88 | 6.67 | 6.77 | 6.58 | 6.55 |
| | | H | 6.63 | 8.87 | 8.03 | 7.98 | 8.10 | 8.13 |
| | | W | 8.06 | 8.82 | 8.26 | 8.30 | 8.10 | 8.20 |
| | | X | 8.27 | 9.23 | 8.26 | 8.53 | 8.11 | 8.18 |
| 4 | C. | A | 5.72 | 6.41 | 6.63 | 5.65 | 6.88 | 5.99 |
| | | B | 6.75 | 7.03 | 6.86 | 6.59 | 6.79 | 6.33 |
| | | C | 6.74 | 6.94 | 6.80 | 6.75 | 6.71 | 6.59 |
| | | D | 6.70 | 7.02 | 6.74 | 6.73 | 6.69 | 6.56 |
| | | E | 6.69 | 7.00 | 6.69 | 6.72 | 6.64 | 6.54 |
| | | F | 6.62 | 6.83 | 6.70 | 6.72 | 6.66 | 6.52 |
| | | G | 6.64 | 6.96 | 6.68 | 6.68 | 6.74 | 6.58 |
| | | H | 6.63 | 8.88 | 8.23 | 8.51 | 8.07 | 8.02 |
| | | W | 8.06 | 8.72 | 8.33 | 8.52 | 8.20 | 8.13 |
| | | X | 8.27 | 9.11 | 8.29 | 8.50 | 8.19 | 8.14 |

*measurements in T₀ were only carried out for one sample set, as no previous storage at three different temperatures of the same sample composition was required

Metabolic activity of the rehydrated samples was assessed by measuring succinic acid, lactic acid and acetic acid concentrations of the approximately 12 month stored rehydrated samples immediately after rehydration (T12m) and 4 hours later (T12m+4 h). At each time point, 1 mL of sample was taken from solution, spun down at 4° C. in an Eppendorf Centrifuge 5415R (Eppendorf, Hamburg, Germany) for 5 min at 13,200 rpm, and then supernatant stored at −20° C.

For further analysis, the supernatants were thawed, filtered through a 0.2 μm filter (VWR Centrifugal filter, modified Nylon, Avantor, Radnor Township, PA), transferred into 2 mL clear glass ID Surestop sample vials (Thermo Fisher Scientific, Waltham, MA) with insert (Target Polyspring Insert Mandrel Point, Thermo Fisher Scientific, Waltham, MA) and inserted into an Agilent FC/ALS Therm. Model G1330B of an Agilent HPLC model 1100 (Agilent Technologies, Santa Clara, CA). The HPLC was equipped with a BioRad Micro-Guard Cation H cartridge and a BioRad Aminex HPX-87H column (BioRad Laboratories, Hercules, CA). The mobile phase was 0.01 N sulfuric acid with a flow rate: 0.6 mL/min applied. Further parameters were: cell temperature: 40° C.; column temperature: 60° C.; run time: 60 min. Compounds were detected with an Agilent DAD (model GI315B) and an Agilent RID (model GI362S) detector, and signal processing accomplished with Agilent LC ChemStation with Open Lab software (C.01.06).

Like indicated already by the lowered pH value (Table 4), the rehydrated cells spring back to life and metabolize the sugar(s) contained in the cryo-protectants into (pH-lowering) organic acids, such as succinic acid, lactic acid and acetic acid (Table 5).

TABLE 5
Concentrations of succinic acid, lactic acid and acetic
acid as measured in the rehydrated samples that were approximately
stored for 12 months at either 37° C., 25° C. or 4° C.,
directly after rehydration (T_{12 m}) or after 4 hours in 14°
C. water (T_{12 m+4 h}).

| | | | succinic acid | | lactic acid | | acetic acid | |
|---|---|---|---|---|---|---|---|---|
| storage | | sam- | [mM] | | [mM] | | [mM] | |
| temp | | ple | T_{12 m} | T_{12 m+4 h} | T_{12 m} | T_{12 m+4 h} | T_{12 m} | T_{12 m+4 h} |
| 37° | C. | A | 0.35 | 0.74 | 0.83 | 1.37 | 0.63 | 0.65 |
| | | B | 0.48 | 0.24 | 0.96 | 0.71 | 1.88 | 1.99 |
| | | C | 0.35 | 0.48 | 0.82 | 1.03 | 2.08 | 3.84 |
| | | D | 0.35 | 0.41 | 0.83 | 0.85 | 1.93 | 2.03 |
| | | E | 0.22 | 0.47 | 0.68 | 0.94 | 2.05 | 2.74 |
| | | F | 0.32 | 0.24 | 0.84 | 0.69 | 2.02 | 2.77 |
| | | G | 0.59 | 0.29 | 1.05 | 0.78 | 3.39 | 2.58 |
| | | H | 0.28 | 0.51 | 0.79 | 1.01 | 2.01 | 2.09 |
| | | W | 0.33 | 0.39 | 0.83 | 0.91 | 2.06 | 1.90 |
| | | X | 0.37 | 0.33 | 0.92 | 0.88 | 2.13 | 1.88 |
| | | S | 0.31 | 0.33 | 0.80 | 0.81 | 1.09 | 1.11 |
| 25° | C. | A | 0.31 | 0.40 | 0.80 | 0.96 | 1.37 | 1.42 |
| | | B | 0.32 | 0.58 | 0.70 | 1.17 | 1.62 | 1.75 |
| | | C | 0.52 | 0.30 | 1.14 | 0.93 | 1.60 | 1.73 |
| | | D | 0.56 | 0.58 | 1.10 | 1.13 | 1.56 | 1.67 |
| | | E | 0.23 | 0.42 | 0.70 | 1.06 | 1.59 | 1.69 |
| | | F | 0.44 | 0.45 | 0.92 | 1.03 | 1.60 | 1.67 |
| | | G | 0.37 | 0.35 | 0.90 | 0.91 | 1.62 | 1.68 |
| | | H | 0.22 | 0.40 | 0.74 | 0.94 | 1.67 | 1.66 |
| | | W | 0.21 | 0.29 | 0.67 | 0.78 | 1.53 | 1.54 |
| | | X | 0.30 | 0.30 | 0.79 | 0.79 | 1.56 | 1.57 |
| | | S | 0.30 | 0.31 | 0.92 | 1.18 | 2.22 | 2.12 |
| 4° | C. | A | 0.25 | 0.51 | 0.73 | 1.32 | 1.53 | 1.83 |
| | | B | 0.38 | 0.44 | 0.92 | 1.24 | 1.58 | 1.95 |
| | | C | 0.39 | 0.29 | 0.96 | 1.02 | 1.58 | 1.92 |
| | | D | 0.23 | 0.26 | 0.75 | 0.94 | 1.55 | 1.88 |
| | | E | 0.51 | 0.25 | 1.10 | 0.94 | 1.58 | 1.89 |
| | | F | 0.40 | 0.36 | 1.05 | 1.22 | 1.70 | 2.01 |
| | | G | 0.69 | 0.25 | 1.26 | 0.88 | 1.54 | 1.76 |
| | | H | 0.32 | 0.36 | 0.78 | 0.91 | 1.50 | 1.51 |
| | | W | 0.29 | 0.33 | 0.79 | 0.88 | 1.52 | 1.65 |
| | | X | 0.35 | 0.32 | 0.82 | 0.82 | 1.48 | 1.51 |
| | | S | 0.28 | 0.30 | 0.86 | 1.16 | 2.27 | 2.27 |

Metabolic activity of the rehydrated samples was assessed in more detail by measuring pH, citric acid, lactic acid and acetic acid concentrations in the solutions of the approximately 2 months stored samples immediately after rehydration (time in bucket=0 min) at varying time intervals for a total of 4 hours (time in bucket=240 min), following the same pH and HPLC measurement protocol as described previously for the metabolic analysis of the approximately 12 month stored rehydrated samples. The time courses for the samples stored at 4° C., 25° C. and 37° C. are depicted in FIG. 49, FIG. 50 and FIG. 51, respectively.

### Example 48: Zeta-Potential of Gram-Positive Aerotolerant Anaerobes. Gram-Negative Anaerobes. And Eukaryotic Cells at 23° C.

This example illustrates the dependence of the Zeta-Potential of (i) the freeze-dried Gram-positive aerotolerant anaerobe Lactobacillus acidophilus NCFM (IFF, New York, NY), (ii) the freeze-dried Gram-positive aerotolerant anaerobe Bifidobacterium animalis susp. lactis B1-04 (ATCC SD5219) from IFF (New York, NY), (iii) the freeze-dried Gram-negative anaerobe Megasphaera elsdenii 1265 (IFF, New York, NY), and (iv) the eukaryotic fungi Saccharomyces cerevisiae from Zenith Yeast Concentrate (AB Biotek, product code: 6400, St. Louis, MO) in dependence of the pH.

(Freeze-) dried product (cells with cryo-protectants) of the four organisms each was dissolved in ultrapure water to give a master solution of approximately 10 OD. Subsequently, 15 mL Eppendorf tubes were filled with 1 mL of 1 M phosphate buffer with different K₂HPO₄/KH₂PO₄ ratios, ultrapure water and 1 mL of the master solution ad 10 mL. Resulting pH in samples was (i) 4.70, 6.25, 6.69 and 8.67 for the Lactobacillus acidophilus NCFM suspensions, (ii) 4.75, 6.24, 6.68 and 8.57 for the Bifidobacterium animalis susp. lactis B1-04 suspensions, (iii) 4.44, 6.28, 6.71, 8.80 for the Megasphaera elsdenii 1265 suspensions, and (iv) 4.60, 6.76, 7.36 and 8.94 for the Zenith Saccharomyces cerevisiae suspensions, respectively, as determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

Measurements of Zeta-potential were carried out as described in Example 27. Results are depicted in FIG. 52. It can be seen that the Zeta-potential of the four organisms do not vary much with the pH of the suspension, ranging from approximately −11 mV for Megasphaera elsdenii 1265 to up to −4 mV for Zenith Saccharomyces cerevisiae suspensions.

### Example 49: Assessing Settling Behavior of Gram-Positive Aerotolerant Anaerobes at 23° C. in 100 mM Phosphate Solution

Settling behavior of Lactobacillus reuteri LAB-1 was evaluated in a TurbiScan Lab Expert (Formulation, Toulouse, France). Changes in transmittance (AT) and backscattering (ABS) were analyzed using a pulsed, near infrared LED at a wavelength of 880 nm.

Freeze-dried powder of Lactobacillus reuteri LAB-1 was dissolved in ultrapure water to give a master solution of approximately 10 OD. Subsequently, 15 mL Eppendorf tubes were filled with 1 mL of either 1 M K₂HPO₄ or 1 M KH₂PO₄ solution, ultrapure water and the corresponding volume of the master solution ad 10 mL, resulting in OD values at λ=600 nm of approximately 1.000 on an Ultrospec 2100 pro UV/VIS spectrophotometer (GE Healthcare/Amersham, Chicago, IL). Resulting pH in samples was either 8.48 or 4.70, respectively, as determined by a Rapid_pH robot from Hudson Robotics (Springfield, NJ). The electrode of the pH robot had been calibrated with standards at pH=4.00 and pH=7.00 with pH=4.00 and pH=7.00 Fisher chemical buffer solutions (Thermo Fisher Scientific, Waltham, MA), respectively.

One sample at a time was loaded into cylinder glass tubes and analyzed for approximately 8 h, in the first 2 hours carrying out a scan every 15 min, hours 3 to 5 every 20 min, and hours 6 to 8 every 30 min. The tube was held at 23° C. The sample height (˜40 mm) was scanned through two different synchronous optical sensors receiving the light transmitted through and backscattered by the sample at an angle of 1800 and 450 to the incident radiation, respectively.

Results with Lactobacillus reuteri LAB-1 in 100 mM K₂HPO₄ are shown in FIG. 53 and in 100 mM KH₂PO₄ solution in FIG. 54. This example demonstrates that Lactobacillus reuteri LAB-1 do not aggregate or flocculate under either high or low pH conditions.

We claim:

1. A composition comprising a) a buffer sufficient to maintain pH of water at about or greater than about 6.5; and b) one or more direct fed microbials (DFMs).

2. The composition of claim 1, further comprising c) a thickening agent

3. The composition of claim 2, wherein the thickening agent is xanthan gum.

4. The composition of any one of claims 1-3, wherein the DFMs are freeze dried or lyophilized.

5. The composition of claim 4, wherein the DFMs comprise a cryoprotectant.

6. The composition of any one of claims 1-3, wherein the composition is hydrated.

7. The composition of any one of claims 1-6, wherein the buffer is sufficient to maintain pH of water in a water line at about or greater than about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

8. The composition of any one of claims 1-7, comprising about 1-2000 mM of the buffer.

9. The composition of claim 8, comprising about 320 mM of the buffer.

10. The composition of claim 7, comprising about 0.001-20 mM of the buffer.

11. The composition of claim 10, comprising about 2.5 mM buffer.

12. The composition of any one of claims 3-11, comprising about 0.1% w/w xanthan gum.

13. The composition of any one of claims 1-12, wherein the buffer comprises one or more buffers selected from the group consisting of potassium phosphate, carbonate, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid.

14. The composition of any one of claims 1-13, wherein the DFM comprises one or more DFM selected from the group consisting of bacteria, algae, and fungi.

15. The composition of claim 14, wherein the bacteria comprises a gram-positive bacteria or a gram-negative bacteria.

16. The composition of claim 14, wherein the fungi comprises a yeast.

17. The composition of any one of claim 14 or claim 15, wherein the bacteria is one or more bacteria selected from the group consisting of a Bacillus spp., a Bifidobacterium spp., a Lactobacillus spp., and a Megasphaera spp.

18. The composition of claim 17, wherein the bacteria is one or more bacteria selected from the group consisting of Bacillus subtilis, Bacillus amyloliquefaciens, Bacillus velezeensis, Bifidobacterium animalis subsp. lactis, Lactobacillus reuteri, Lactobacillus acidophilus, and Megasphaera elsdenii.

19. The composition of any one of claims 6-18, wherein the DFMs exhibit decreased settling and/or improved survival in the hydrated composition compared to identical DFMs that are not in a hydrated composition comprising a buffer sufficient to maintain pH of water at about or greater than about 6.5.

20. The composition of any one of claims 1-19, wherein the water is selected from the group consisting of a municipal water source, well water, surface water, and collected rain water.

21. The composition of claim 20, wherein the water is supplemented with chloramine or other chlorine-based antimicrobial.

22. The composition of claim 21, further comprising d) means for inactivating chloramine or chlorine present in the water.

23. The composition of any one of claims 1-22, further comprising e) a wetting agent and/or dispersing agent.

24. The composition of any one of claims 1-23, wherein the temperature of the composition is less than about 15° C.

25. A method for preparing a composition for maximizing delivery of live direct fed microbial (DFM) cells through a water line comprising mixing i) a buffer sufficient to maintain pH of water at about or greater than about 6.5; and ii) one or more direct fed microbials (DFMs) with water.

26. The method of claim 25, wherein the DFMs are freeze dried or lyophilized.

27. The method of claim 25, wherein the DFMs comprise a cryoprotectant.

28. The method of any one of claims 25-27, wherein the water is selected from the group consisting of a municipal water source, well water, surface water, and collected rain water

29. The method of any one of claims 25-18, further comprising mixing iii) a thickening agent with water.

30. The method of any one of claims 25-29, wherein the mixing results in a final concentration of about 1-2000 mM of the buffer.

31. The method of claim 30, wherein the mixing results in a final concentration of about 320 mM of the buffer.

32. The method of any one of claims 29-31, comprising about 0.1% w/w thickening agent.

33. The method of any one of claims 29-32, wherein the buffer is sufficient to maintain pH of water in a water line at about or greater than about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

34. The method of any one of claims 25-33, wherein the buffer is one or more buffers selected from the group consisting of potassium phosphate, carbonate, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid.

35. The method of any one of claims 25-34, wherein the DFM is one or more DFM selected from the group consisting of bacteria and fungi.

36. The method of claim 35, wherein the bacteria is a gram-positive bacteria or a gram-negative bacteria.

37. The method of claim 35, wherein the fungi is a yeast.

38. The method of any one of claim 35 or claim 36, wherein the bacteria is one or more bacteria selected from the group consisting of a Bacillus spp., a Bifidobacterium spp., a Lactobacillus spp., and a Megasphaera spp.

39. The method of claim 38, wherein the bacteria is one or more bacteria selected from the group consisting of Bacillus subtilis, Bacillus amyloliquefaciens, Bacillus velezensis, Bifidobacterium animalis subsp. lactis, Lactobacillus reuteri, Lactobacillus acidophilus, and Megasphaera elsdenii.

40. The method of any one of claims 25-39, wherein the water is supplemented with chloramine or other chlorine-based antimicrobial.

41. The method of claim 40, further comprising mixing iv) means for inactivating chloramine or chlorine present in the water with water.

42. The method of any one of claims 25-41, further comprising mixing v) a wetting agent and/or dispersing agent with water.

43. The method of any one of claims 25-42, wherein the temperature of the composition is less than about 15° C.

44. A composition produced by the method of any one of claims 25-43.

45. A method for delivering one or more direct fed microbials (DFMs) to a subject comprising administering the composition of any one of claims 1-24 or claim 44 through a water line over a distance, wherein the DFMs are delivered to the subject when the subject drinks from the water line.

46. The method of claim 45, wherein the DFMs exhibit decreased settling and/or survival in the water line compared to identical DFMs that are not administered in the composition of any one of claims 1-24 or claim 44.

47. The method of claim 45 or claim 46, wherein the water line comprises nipple drinkers.

48. The method of any one of claims 45-47, wherein the distance is between about 20 meters to about 200 meters.

49. The method of claim 48, wherein the water line further comprises between about 20-100 meters of piping that lacks nipple drinkers.

50. The method of any one of claims 45-49, wherein the subject is poultry, swine, or a ruminant.

51. The method of claim 50, wherein the poultry is a chicken or a turkey.

52. The method of claim 50 or claim 51, wherein the chicken is a layer or a broiler.

53. The method of claim 50, wherein the swine is a piglet, a grower, a finisher, or a sow.

54. The method of claim 50, wherein the ruminant is a beef cattle, a milk cattle, or a veal cattle.

55. A kit comprising a) a buffer sufficient to maintain pH of water at about or greater than about 6.5; and b) one or more direct fed microbials (DFMs).

56. The kit of claim 55, further comprising c) a thickening agent.

57. The kit of claim 56, wherein the thickening agent is xantham gum.

58. The kit of any one of claims 55-57, wherein the buffer is sufficient to maintain pH of water in a water line at about or greater than about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

59. The kit of any one of claims 55-58, comprising 3-500 g of the buffer.

60. The kit of any one of claims 55-59, wherein the buffer comprises one or more buffers selected from the group consisting of potassium phosphate, carbonate, TAPS ([tris(hydroxymethyl)methylamino]propanesulfonic acid), Bicine (2-(bis(2-hydroxyethyl)amino)acetic acid), Tris (tris(hydroxymethyl)aminomethane, or 2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid.

61. The kit of any one of claims 55-60, wherein the DFM comprises one or more DFM selected from the group consisting of bacteria, algae, and fungi.

62. The kit of claim 61, wherein the bacteria comprises a gram-positive bacteria or a gram-negative bacteria.

63. The kit of claim 61, wherein the fungi comprises a yeast.

64. The kit of any one of claim 61 or claim 62, wherein the bacteria is one or more bacteria selected from the group consisting of a Bacillus spp., a Bifidobacterium spp., a Lactobacillus spp., and a Megasphaera spp.

65. The kit of claim 64, wherein the bacteria is one or more bacteria selected from the group consisting of Bacillus subtilis, Bacillus anyloliquefaciens, Bacillus velezensis, Bifidobacterium animalis subsp. lactis, Lactobacillus reuteri, Lactobacillus acidophilus, and Megasphaera elsdenii.

66. The kit of any one of claims 55-65, further comprising d) means for inactivating chloramine or chlorine present in water.

67. The kit of any one of claims 55-66, wherein the kit components are packaged in a sachet, water-dissolvable tablets, water-dissolvable pods (e.g. powder distributed in cold water soluble PVA film bags) or in water-dissolvable (soft gel) capsules (e.g. in pullulan capsules).

68. The kit of any one of claims 66-67, further comprising e) written instructions for combining the kit components with water for water line delivery.

69. The kit of any one of claims 55-68, wherein the DFMs are freeze dried or lyophilized.

70. The kit of any one of claim 69, wherein the DFMs further comprise a cryoprotectant.

71. The kit of any one of claims 55-70, further comprising f) a wetting and/or dispersing agent.
